(19) United States
(12) Patent Application Publication
(10) Pub. No.: US 2007/0275374 A1
(43) Pub. Date: Nov. 29, 2007

(54) Methods For Idendifying Drug Targets And Modulators Of Neurons and Compositions Comprising The Same

(75) Inventors: Jan Markus Grimm, Menlo Park, CA (US); Arnon Rosenthal, Woodside, CA (US)

Correspondence Address: MORRISON & FOERSTER LLP, 755 PAGE MILL RD, PALO ALTO, CA, 94304-1018, US

(73) Assignee: RINAT NEUROSCIENCE CORP., SOUTH SAN FRANCISCO CALIFRONIA (CA)

(21) Appl. No.: 10/547,955

(22) PCT Filed: Mar. 17, 2004
(86) PCT No.: PCT/US04/08327

§ 371 Date: Oct. 16, 2006

Related U.S. Application Data
(60) Provisional application No. 60/455,520, filed on Mar. 17, 2003.

(51) Int. Cl. C12Q 1/68; C12M 3/00; G01N 33/53

(52) U.S. Cl. 435/6; 435/7.8; 435/287.2

(57) ABSTRACT

The invention provides methods of identifying drug targets in dopaminergic and/or noradrenergic neurons and to the drug targets identified by such methods. This invention also provides a method of screening for agents that modulate dopaminergic neurons and/or noradrenergic neuron activity, function and/or drug target expression, and agents that bind drug targets and to kits for use in the methods described herein.

### CROSS RELATED APPLICATIONS

This application claims the benefit under 35 U.S.C. §119(e) to U.S. Patent Application No. 60/455,520 filed Mar. 17, 2003, the contents of which are herein incorporated by reference in their entirety.

### STATEMENT REGARDING FEDERALLY SPONSORED RESEARCH OR DEVELOPMENT

Not applicable.

### FIELD OF THE INVENTION

This application is in the field of neuroscience, in particular, this invention relates to methods for identifying polynucleotide and polypeptide drug targets in dopaminergic and noradrenergic neurons, to drug targets identified by the methods described herein and to methods for screening for modulators of dopaminergic and noradrenergic neurons and compositions for use in the methods described herein.

### BACKGROUND OF THE INVENTION

Dysfunction of midbrain dopaminergic and hindbrain noradrenergic neurons is central to the development of several neurological and psychiatric diseases or disorders. Midbrain dopaminergic neurons and their projections fall into three major systems (Airaksinen, M. S., et al (1997). Eur J Neurosci 9, 120-127). Nigrostriatal dopaminergic neurons reside largely within the substantia nigra pars compacta, project to the putamen and caudate nucleus. They comprise a key component of the voluntary motor system, and their degeneration leads to the development of Parkinson's disease, one of the most common neurodegenerative disorders with a prevalence of approximately 1% in the population over the age of 65 with estimated costs exceeding $25 Billion each year in the United States alone (Abeliovich, A. et al (2000) Neuron 25:235-252).

Mesolimbic dopaminergic neurons reside in the ventral tegmental area and project to the amygdala, endorinal cortex and septum. These neurons influence emotional balance and addictive behavior (e.g. alcohol and cocaine). The abuse of recreational drugs is common in our society and has a major impact on our health care system with estimated costs exceeding $245.7 billion in 1992 in the US alone (www.nida.nih gov/Infofax/costs.html)) (Ambrozi, L. et al, (1976). Br J Pharmacol 58, 423P-424P). Mesocortical dopaminergic neurons reside in the ventral tegmental area and project to the neocortex in the frontal area. These neurons influence motivation, attention and planning. Hyperactivity of this pathway has been associated with schizophrenia. Approximately 1% of the population experience at least one schizophrenic episode at some time in their life with estimated annual costs—$32.5B in the (httl2://www.schizophrenia.com/newsletter/buckets/intro.html).

Incertohypothalamic dopamine neurons located in the most rostral portion of the medial zona incerta were originally described as the A13 tyrosine hydroxylase-containing group (Dahlstrom A, Fuxe K. (1965) Experientia. Jul 15;21(7):409-10.). Anatomical studies in rats suggest an involvement of the zona incerta in motor and oculomotor functions due to its connections with the pedunculopontine nucleus, the substantia nigra pars reticulata and the superior colliculus. Stereotactic surgery aimed at destroying the zona incerta area in Parkinsonian patients has been shown to relieve the motor symptoms, suggesting that structure might have a role in pathophysiology of the disease.

The largest collection of noradrenergic (NA) neurons in the central nervous system (CNS) is found in the locus coeruleus (LC). These neurons reside in the ventro-lateral region of the first hindbrain rhombomere and project to regions throughout the CNS. Their degeneration is associated with Parkinsons and Alzheimers disease (Chan-Palay, V. (1991). Alterations in the locus coeruleus in dementias of Alzheimers and Parkinsons disease. In Neurobiology of the Locus Coeruleus: Progress in Brain Research, C. D. Barnes and O. Pompeiano, eds. (Amsterdam: Elsevier Science Publishers)), whereas their abnormal function is thought to play a role in depression, sleep disorders (Siegel, M. J. (1999) Cell 98: 409-412.), and schizophrenia (Brier, B. et al., (1998). Norepinephrine and Schizophrenia: a new hypothesis for antipsychotic drug addiction. In Catecholamines: Bridging Basic Science with Clinical Medicine, Goldstein, D. S., Eisenhofer, G., and McCarty, R., eds. (Academic Press), pp 785-788.).

Patients with Parkinson's disease suffer from impaired motor function characterized by rhythmic tremor, inability to initiate and complete routine movement, muscle rigidity, postural instability and paucity of facial expression. The clinical symptoms are preceded by a selective loss of pigmented dopamine-producing neurons in the substantia nigra and ventral tegmental area in combination with a varying decay of the noradrenergic (locus coeruleus), cholinergic forebrain (nucleus basalis of Meynert) and serotoninergic (dorsal raphe nuclei) systems. The disease occurs sporadically in most cases, and the cause of cell death is not known, although viral infections, environmental toxins and oxidative stress induced by dopamine metabolites have been proposed. With loss of these neurons, excessive inhibitory stimuli are sent from the basal ganglia through the globus pallidus to the thalamus, leading to a decrease in the motor cortex activity and to the negative symptoms of Parkinson's disease; akinesia, bradykinesia and rigidity. In addition the loss of feed back loop between the nigral dopaminergic neurons and the thalamus leads to the release of spontaneous periodical impulse in the thalamus, which are responsible in part to the characteristic tremors (Deuschl, G. et al. (2000) J Neurol 247: Suppl 5, V33-48).

Not all midbrain dopaminergic neurons are equally susceptible to neurodegeneration in Parkinson's disease. Dopaminergic neuronal loss is most severe in the substantia nigra pars compacta while cells in the ventral tegmental area are less vulnerable (Hirsch et al., (1988) Nature 334:345-348). Within the substantia nigra pars compacta, the anatomical location and the expression of a variety of markers are associated with increased susceptibility to degeneration and loss. Neuronal loss tends to be greatest in the ventrolateral tier, followed by the ventromedial tier and dorsal tier (Farneley and Lees (1991) Brain 114 (Pt 5): 2283-2301.). This pattern of cell loss is specific to Parkinson's disease; it is the opposite of that seen in normal aging and differs from patterns found in striatonigral degeneration and progressive supranuclear palsy. It results in a regional loss of striatal dopamine, most prominently in the dorsal and intermediate subdivisions of the putamen, a process that is believed to account for akinesia and rigidity. This pattern of cell loss correlates with the expression level of dopamine transporter mRNA (Uhl et al., (1994) Ann. Neurol. 35: 494-498). Neuromelanin-containing neurons are more susceptible to neurodegeneration while non-pigmented neurons are largely spared (Hirsch et al., 1988). Neuromelanin first appears in dopaminergic neurons within 3 years of birth and increases with age. Neuromelanin is suspected to bind neurotoxins such as MPTP, paraquat or toxic metals or itself catalyze the production of toxic free radicals, providing a toxin pool within the pigmented neurons. It is, however, unlikely that neuromelanin is the sole causal factor for Parkinson's pathogenesis as it is accumulated in all humans with age. Differential expression of the calcium-binding proteins calbindin-D 28 kD and calretinin in a subset of midbrain dopaminergic neurons has been shown to be associated with neuroprotective advantage in Parkinson's disease (Tan et al., (2000) Brain Res. 869:56-68). The calcium-binding proteins are found in the majority neurons in the ventral tegmental area, whereas in the substantia nigra less than 40% of the cells contained either calcium-binding protein. Gene inactivation studies in mice have shown that calbindin is not, however, causally involved in conferring resistance to neurotoxins and thus might only be used as a marker for less vulnerable cells (Airaksinen et al., (1997) Eur. J. Neurosci. 9:120-127).

In contrast to mesencephalic dopaminergic neurons, neurodegeneration of dopaminergic neurons in the hypothalamus is much less pronounced in Parkinson's disease. Different studies have revealed either none or only very limited loss of dopamine cells in several hypothalamic nuclei in Parkinson's brains (Purba et al., (1994) Neurology Jan;44(1):84-9; Matzuk et al., 91985) Ann Neurol 5:552-5).

Quantitative analysis of degeneration of pigmented neurons in the locus coeruleus revealed that about 70% of the noradrenergic neurons are lost in Parkinson's disease. Cells in the rostral and caudal part are equally affected by the disease, in contrast to more pronounced loss of cells in the rostral part in the locus coerules that has been observed during normal ageing (Chan-Palay V, and Asan E. (1989) Comp Neurol. 287(3):373-92; Bertrand E. et al (1997) Folia Neuropathol 35(2):80-6).

The most accepted theory for the development of Parkinson's Disease (PD) involve the abnormal aggregation of a presynaptic protein designated alpha-synuclein, a 14 kd protein that was initially isolated from cholinergic nerve terminals of the Torpedo ray electric organ (Maroteaux et al. (1988) J. Neurosci. 8: 2804-2815). Parkinson's Disease brain pathology is typified by the presence of abnormal protein aggregates, termed Lewy bodies, and selective loss of dopamine (DA) neurons. Alpha-synuclein appears to be the major protein component of these intra-cytoplasmic deposits in sporadic and familial forms of the disease (Mezey et al. (1998) Nature Med. 4:755-756; Spillantini et al. (1998) Proc. Natl. Acad. Sci (USA) 95:6469-6473). Direct evidence for the involvement of alpha-synuclein in Parkinson's Disease was provided by genetic studies of patients with rare, dominantly inherited variants of this disorder. Two independent pathological mutations have been described, a change from alanine to threonine at position 53 in Italian-American and Greek families (Polymeropoulos et al. (1997) Science 276:2045-2047), and a change from alanine to proline at position 30 in a family of German origin (Kruger et al. (1998) Nat. Genet. 18(2):106-8). These mutant proteins display a propensity to form Lewy body-like fibrils in vitro (Conway et al. (1998) Nature Med. 4: 1318-1320). Moreover, expression of the human alpha-synuclein mutation in transgenic mice results in Parkinson's Disease-like symptoms (Betarbet et al. (2002) Bioessays 24(2):308-318), while ablation of alpha-synuclein results in abnormal regulation of dopamine release (Abeliovich et al. (2000) Neuron 25:235-252). Unfortunately, despite the strong evidence for the involvement of alpha-synuclein in Parkinson's Disease its mechanism of its action and the genes involved in the process had not been yet identified.

Many different therapeutic approaches have been used in an attempt to counteract or compensate for the neural or chemical deficiencies that underline Parkinson's disease. The most effective treatment currently available is L-Dopa administration. L-Dopa is a precursor for dopamine, which crosses the blood brain barrier, and is taken up by the remaining dopaminergic neurons, converted to dopamine, which is secreted in the appropriate targets. L-Dopa compensates for the reduction in the level of the endogenous dopamine, increases the levels of dopamine in the striatum, and leads to a reversal or amelioration of the akinesia, bradykinesia and rigidity (Ambrozi et al. (1976) Br. J. Pharmacol. 58: 423P-424P). Unfortunately, it is not effective in reducing the tremors, nor does it slow the disease progression. Furthermore, after several years of treatment, L-Dopa leads to severe side effects and is no longer efficacious. Surgical lesions in the globus pallidus (pailidotomy) and electric stimulation of the subthalamic nuclei have been tried (both aimed at reducing the hyperactivity of the globus pallidus resulting from loss of dopaminergic neurons). However, although pallidotomy and electrical stimulation show promise in reducing akinesia and bradykinesia, especially akinesia that is induced by L-Dopa in advanced Parkinson's patients, they are not consistently effective in reducing the tremors. In addition, many symptoms recur after only a few years. A third therapeutic approach is grafting of dopamine-producing cells derived from fetal midbrain tissues, adrenal medulla or carotid body. However, in a recent large clinical trial with human fetal neurons, no consistent therapeutic benefits were observed and some patients experienced severe side effects.

Schizophrenia is one of the most common mental illnesses, affecting about 1% of the population, with an estimated cost to society of $32.5 billion per year in the US (U.S. Census Bureau and American Psychiatric Association). Schizophrenia is characterized by a constellation of distinctive symptoms that include thought disorder, delusions, and hallucinations. Thought disorder is the diminished ability to think clearly and logically. Often it is manifested by disconnected and nonsensical language. Delusions are common among individuals with schizophrenia, and are frequently paranoid or grandiose in nature. Hallucinations can be auditory, visual, olfactory or tactile. Most often they take the form of voices that may describe the person's actions, warn him of danger or tell him what to do. In addition, schizophrenics tend to be socially withdrawal, lack emotion and expression, and have reduced energy, motivation and activity. Sometimes schizophrenics exhibit catatonia where they become fixed in a single position for a long period of time. The first psychotic episode generally occurs in late adolescence or early adulthood, and often necessitates hospitalization where antipsychotic medication can commence under close supervision. Some persons with schizophrenia recover completely, and many others improve to the point where they can live independently, often with the maintenance of drug therapy. However, approximately 15 percent of people with schizophrenia respond only moderately to medication and require extensive support

The proposal that schizophrenia is caused by an overactive dopamine system is based on the pharmacological findings that the drugs stimulating central dopamine receptors can produce a disorder indistinguishable from schizophrenia, and that anti-psychotic drugs block dopamine receptors (Davis et al. (1991) Am. J. Psychiatry 148: 1474-1486). However, whereas anti-psychotics block dopamine receptor activation soon after administration, therapeutic benefits are only seen after several weeks, suggesting that the primary defect in this disease may lie downstream of dopaminergic signaling. Thus, it is likely that other effectors have to be identified to address the cause of schizophrenia The need for more effective anti-psychotic drugs not only stems from the limited effectiveness of such drugs in an appreciable number of schizophrenic patients but from the many side effects of such drugs. Because these drugs block dopamine action, not surprisingly one of the most serious side effects of these drugs is the appearance of Parkinson's disease-like symptoms: tremor, muscle rigidity, loss of facial expression. Other side effects include dystonia, restlessness and tardive dyskinesia—involuntary, abnormal movements of the face, mouth, and/or body, which develop in about 25-40% of patients who take antipsychotic mediations for several years (http://www.schizophrenia.com/newsletter/buckets/intro htmi).

The role of noradrenergic neurotransmission in normal cognitive functions has been extensively investigated, however, the involvement in the cognitive impairment associated with schizophrenia has not been as intensively considered. The evidence of noradrenergic dysfunction occurring concomitantly with dopamine dysfunction in schizophrenia supports therapeutic approaches using noradrenergic drugs in combination with neuroleptics to enhance the treatment of cognitive impairment. Compared to typical antipsychotics (e.g. haloperidol), the newer atypical antipsychotics (e.g. risperidone and olanzapine) have greatly improved efficacy and exhibit less extrapyramidal motor side-effects. Acute treatment with atypical antipsychotics has been shown to induce c-Fos expression and transmitter release of locus coeruleus neurons (Ohashi, K et al. (2000) Neuropsychopharmacology, 23:162-9; Dawe, G S et al. (2001) Biological Psychiatry, 50:510-20).

Addiction is typically a chronic, relapsing brain disorder in which compulsive drug procurement and use dominate an individual's motivation (Tecott and Heberlein (1998) Cell 95:733-735). Drugs of abuse have been hypothesized to produce their rewarding effects by neuropharmacological actions on a common brain reward circuit of which the mesolimbic dopaminergic neurons are a key component. Natural rewards (e.g., sex and food) as well as addictive substances activate this reward circuit. Heroin, for example, increases the firing rate of dopaminergic neurons, whereas cocaine inhibits reuptake of dopamine. In addition to their acute effects, repeated use of psychomotor stimulants like cocaine and opiates like heroin produces changes in the mesolimbic dopamine system. Specifically, repeated use of cocaine or heroin can deplete dopamine from this system (Kish et al (2001) Neuropsychopharmacology 24:561-567) These dopamine depletions may cause normal rewards to lose their motivational significance. At the same time, the mesolimbic dopamine system becomes even more sensitive to pharmacological activation by psychomotor stimulants and by opiates (i.e., sensitization develops). These neuroadpative changes are probably critical for producing an addiction (De Vries et al. (1999) Psychopharmacology (Berl) 143:254-260). Substances that activate the mesolimbic dopamine system without producing these neuroadaptive effects are probably not truly addictive.

Noradrenergic neurons in the LC express high levels of opioid receptors and plays a role in several effects of opioids, such as opioid dependence and withdrawal (Nestler E J et al (1994) Brain Res Bull 35:521-528; Nestler E J et al. (1997) Science 278:58-63). Systemic or intracoerulear administration of opioids, such as morphine, has been shown to have an inhibitory action on spontaneous LC neuronal activity (Korf J. et al. (1974) Eur J Pharmacol. 25:165-169). More recent studies suggest that administration of morphine does not simply decrease firing rates of LC neurons, but that it induces long-lasting synchronous oscillatory discharges in a subpopulation of LC neurons. These discharges may result in a facilitation of noradrenaline release in the widespread LC target areas (Zhu H and Zhou W. J (2001) Neurosci (21)21: RC179).

Dopaminergic and noradrenergic neurons have not been isolated free of other neurons and glial cells and only a few of the genes that are specifically expressed by these neurons have been identified. Identification of such genes (e.g., gene expression profiles) in, for example, in specific subsets of dopamine cells in Parkinson's disease that show different vulnerability will facilitate the identification key regulators that are involved in neuronal survival and potential drug targets for Parkinson's disease. Likewise, in the case of schizophrenia and drug addiction, the identification of genes that are specifically expressed in certain dopaminergic and noradrenergic neurons will provide novel candidates to target in the disease or addiction and a better understanding of the etiology of the disease or addiction. As dopaminergic and noradrenergic neurons are implicated in a variety of neurological diseases and disorders, there is substantial interest in identifying drug targets in these neurons and agents capable of modulating their activity. This invention provides such methods, drug targets and compositions for use in the methods.

All references cited herein, including patent applications and publications, are incorporated by reference in their entirety. References include database sequences.

### SUMMARY OF THE INVENTION

The invention relates, in general, to a method of identifying polynucleotide drug targets or polypeptide drug targets in dopaminergic and/or noradrenergic neurons and to the polynucleotide or polypeptide drug targets identified by such methods. This invention also provides a method of screening for agents that modulate neuron activity and/or function and/or gene expression via the polynucleotide or polypeptide drug targets and/or agents that bind to the polynucleotide or polypeptide drug targets identified by the methods described herein and to kits for use in the methods described herein.

In one aspect, the invention provides a method of identifying candidate drug targets in a dopaminergic and/or noradrenergic neuron comprising: (a) identifying and/or isolating a population of dopaminergic neurons (e.g., dopaminergic neurons in the substantia nigra pars compacta) and/or noradrenergic neurons; (b) evaluating the expression of one or more polynucleotides in the population of neurons, wherein the one or more polynucleotides and/or the one or more encoded polypeptides are candidate drug targets. The method may further comprise evaluating the expression of one or more polynucleotides in step (b) relative to a control population of neurons (e.g., whole brain).

In another aspect this invention relates to a method of identifying candidate drug targets in a population of dopaminergic or noradrenergic neurons comprising evaluating the expression of one or more polynucleotides in a dopaminergic or noradrenergic neuron population, wherein the one or more polynucleotides and their corresponding polypeptides are candidate drug targets.

In yet another embodiment this invention provides polynucleotide drug targets identified by the methods described herein and/or polypeptide drug targets identified by the methods described herein or combinations thereof and compositions and/or kits comprising the same.

In yet another aspect of the invention, microarrays comprising the polynucleotides and/or polypeptides of the invention are provided.

Yet another aspect of the invention relates to an antibody directed against the polypeptides of the invention. In some aspects the antibody modulates the activity and/or function of the polypeptides.

In yet another aspect, this invention provides a method of assessing the ability of a candidate agent to modulate dopaminergic and/or noradrenergic neuron activity and/or function comprising: (a) contacting a population of dopaminergic and/or noradrenergic neurons expressing one or more drug targets (e.g., polynucleotide and/or polypeptide drug targets) with a candidate agent and (b) measuring the level of expression of the one or more drug targets in the population of dopaminergic and/or noradrenergic neurons, wherein an alteration of the level of expression of the one or more drug targets indicates the ability of the candidate agent to modulate dopaminergic and/or noradrenergic neuron activity and/or function and/or the therapeutic potential of the candidate agent for treating one or more diseases or disorders associated with dopaminergicand/or noradrenergic neuron activity or one or more symptoms associated with dopaminergicand/or noradrenergic neuron activity.

In one aspect, the method of assessing the ability of a candidate agent to modulate dopaminergic and/or noradrenergic neuron activity and/or function comprises measuring the level of expression of the gene transcripts for the one or more drug targets (e.g., RNA). In another aspect, the method of assessing the ability of a candidate agent to modulate dopaminergic and/or noradrenergic neuron activity and/or function comprises measuring the level of the polypeptide drug target.

In one embodiment this invention provides a method of screening for candidate agents that modulate dopaminergic and/or noradrenergic neuron activity, wherein the population of dopaminergic and/or noradrenergic neurons comprise, for example, nigrostriatal dopaminergic neurons in the substantia nigra pars compacta, mesolimbic and mesocotical dopaminergic neurons in the ventral tegmental area, hypothalamic dopaminergic neurons in the zona incerta (A13 group) and noradrenergic neurons in the locus coeruleus.

In another aspect this invention provides a method of assessing the ability of a candidate agent to modulate dopaminergic and/or noradrenergic neuron activity and/or function comprising: (a) contacting a population of dopaminergic and/or noradrenergic neurons expressing one or more drug targets (e.g., polynucleotide and/or polypeptide drug targets) with a candidate agent and (b) evaluating the activity and/or function of the population of dopaminergic and/or noradrenergic neurons, wherein an alteration in the dopaminergic and/or noradrenergic neuron activity indicates the therapeutic potential of the candidate agent for treating one or more diseases or disorders associated with dopaminergic and/or noradrenergic neuron activity or one or more symptoms associated with dopaminergic and/or noradrenergic neuron activity.

In one embodiment this invention provides a method of screening for candidate agents that modulate dopaminergic and/or noradrenergic gene expression, wherein the population of dopaminergic and/or noradrenergic neurons comprise, for example, nigrostiatal dopaminergic neurons in the substantia nigra pars compacta, mesolimbic and mesocotical dopaminergic neurons in the ventral tegmental area, hypothalamic dopaminergic neurons in the zona incerta (A13 group) and noradrenergic neurons in the locus coeruleus.

In yet another aspect of this invention, a method of assessing the ability of a candidate agent to bind to one or more of the polynucleotide and/or polypeptide drug target identified by the methods described herein is provided.

Yet another aspect of this invention provides a method of staining nerve cells and maximizing isolation and/or recovery of polynucleotides (e.g., RNA) for use in the methods described herein.

Another aspect of this invention provides kits for use in the methods described herein.

### BRIEF DESCRIPTION OF THE FIGURES

FIG. 1. Impact of immunostaining procedure on RNA integrity. (a) Profile of RNA extracted from a cryosection from a fresh rat brain analyzed with the Agilent bioanalyzer. (b) RNA profile after immunostaining with our rapid staining protocol. The RNA is well preserved and shows a high 28S to 18S RNA ratio. (c) RNA content after immunostaining protocol with longer incubation with primary antibody (6 min) without addition of RNAse inhibitors to all buffers. The RNA is severely compromised and not suitable for amplification and microarray analysis.

FIGS. 2A-2B. FIG. 2A shows identification of dopaminergic neurons in the substantia nigra pars compacta (SNc), the ventral tegmental area (VTA), the zona encerta (A13) and noradrenergic neurons in the locus coeruleus (LC). Rat brain sections were stained for tyrosine hydroxlase with the rapid staining protocol described herein. FIG. 2B. Microdissection of immunostained tyrosine hydroxylase positive neurons from the substantia nigra pars compacta.

FIG. 3. Integrity of RNA extracted from 3 different autopsy samples of the human substantia nigra. Sample a shows reasonable preservation of RNA while sample c contains degraded RNA that is not suitable for amplification and microarray analysis.

FIG. 4. Microdissection of single pigmented neurons from the human subtanita nigra compacta.

FIGS. 5A-5B. Drug targets identified in zona encarta A13 neurons. Accession numbers of human orthologs are for TIGR Human Gene Index (THC numbers) and Genebank. The listed drug targets are expressed at least 8 fold higher relative to whole brain. The sequences referenced in this Figure are herein incorporated by reference in their entirety.

FIG. 6A-6B. Drug targets identified in locus coeruleus (LC) neurons. Accession numbers of human orthologs are for TIGR Human Gene Index (THC numbers) and Genebank. The listed drug targets are expressed at least 8 fold higher relative to whole brain. The sequences referenced in this Figure are herein incorporated by reference in their entirety.

FIG. 7A-7B. Drug targets identified in ventral tegmental area (VTA) neurons. Accession numbers of human orthologs are for TIGR Human Gene Index (THC numbers) and Genebank. The listed drug targets are expressed at least 8 fold higher relative to whole brain. The sequences referenced in this Figure are herein incorporated by reference in their entirety.

FIG. 8A-8B. Drug targets identified in substantia nigra (SN)neurons. Accession numbers of human orthologs are for TIGR Human Gene Index (THC numbers) and Genebank. The listed drug targets are expressed at least 8 fold higher relative to whole brain. The sequences referenced in this Figure are herein incorporated by reference in their entirety.

FIG. 9. Drug targets with higher expression in SN neurons relative to VTA neurons. Accession numbers of human orthologs are for TIGR Human Gene Index (THC numbers) and Genebank. The sequences referenced in this Figure are herein incorporated by reference in their entirety.

FIG. 10. Drug targets with higher expression in VTA neurons relative to SN neurons. Accession numbers of human orthologs are for TIGR Human Gene Index (THC numbers) and Genebank. The sequences referenced in this Figure are herein incorporated by reference in their entirety.

FIG. 11A-11C. Drug targets identified in human SN neurons. The listed drug targets are expressed at least 8 fold higher relative to whole brain. The sequences referenced in this Figure are herein incorporated by reference in their entirety.

FIG. 12. Drug targets identified in human LC neurons. The listed drug targets are expressed at least 8 fold higher relative to whole brain. The sequences referenced in this Figure are herein incorporated by reference in their entirety.

FIG. 13A-13C. Drug targets whose transcripts are expressed at least 4 fold higher in all catecholaminergic (CA) neurons relative to whole brain. The sequences referenced in this Figure are herein incorporated by reference in their entirety.

FIG. 14A-14T. Drug targets whose transcripts are differentially expressed in subsets of catecholaminergic (CA) neurons. Genes were filtered based on expression level relative to the whole brain reference (>4-fold higher or lower in 3/16 experiments) and transcripts with significant differences in expression between at least two cell groups were selected by multiclass SAM with a false discovery rate of <1%. The resulting set of genes and the experimental samples were grouped based on their similarities of gene expression by supervised hierarchical clustering (Pearson correlation, average linkage). Shaded areas indicate gene clusters. The sequences referenced in this Figure are herein incorporated by reference in their entirety.

FIG. 15A-15F. Drug targets whose transcripts are differentially expressed between SN and VTA neurons. Two-class significance analysis with a false discovery rate cut-off of <1% was used to identify the genes. The sequences referenced in this Figure are herein incorporated by reference in their entirety.

FIG. 16. In situ hybridization analysis with probes for tyrosine hydroxylase, hypothetical 38.5 kd protein and ZIP-4 demonstrates specific expression in the SN and VTA.

### DETAILED DESCRIPTION OF THE INVENTION

Throughout the disclosure, various publications, patents and published patent specifications are referenced by an identifying citation. The disclosure of these publications, patents and published patent specifications are hereby incorporated by reference into the present disclosure to more fully describe the state of the art to which this invention pertains.

Definitions

The practice of the present invention will employ, unless otherwise indicated, conventional techniques of immunology, molecular biology, microbiology, cell biology and recombinant DNA. [See, e.g., Sambrook, et al. MOLECULAR CLONING: A LABORATORY MANUAL, 3rd edition (2001); SHORT PROTOCOLS IN MOLECULAR BIOLOGY (F. M. Ausubel, et al. eds., 5^{th} Edition (1995); the series METHODS IN ENZYMOLOGY (Academic Press, Inc.); “PCR: A PRACTICAL APPROACH” (M. MacPherson, et al., IRL Press at Oxford University Press (1991); PCR 2: A PRACTICAL APPROACH (M. J. MacPherson, B. D. Hames and G. R. Taylor eds (1995)); ANTIBODIES, A LABORATORY MANUAL (Harlow and Lane, eds (1988)); and CULTURE OF ANIMAL CELLS (R. I. Freshney, ed. 4^{th} Edition (2000)).

As used in the specification and claims, the singular form “a”, “an” and “the” include plural references unless the context clearly dictates otherwise. For example, the term “a gene” includes more than one or a plurality of genes, including mixtures or fragments thereof.

The term “polynucleotide” refers to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes, for example, single-, double-stranded and triple helical molecules, a gene or gene fragment, exons, introns, mRNA, tRNA, rRNA, iRNA, ribozymes, cDNA, recombinant polynucleotides, branched polynucleotides, plasmids, vectors, isolated DNA of any sequence, isolated RNA of any sequence, nucleic acid probes, and primers. A nucleic acid molecule may comprise, for example, naturally occurring nucleic acid molecules, synthetic or modified nucleic acid molecules.

The term “polypeptide” refers to polymeric forms of amino acids of any length and may include, but is not limited to, naturally occurring or modified amino acids.

The term “expression” includes production of a gene transcript and/or polypeptide.

The term “dopaminergic disease or disorder” refers to a disease or disorder in which the dopaminergic neurons are effected, involved and/or implicated. By way of example and not limitation such diseases or disorders include Parkinson's disease, schizophrenia or drug addition.

The term “noradrenergic disease or disorder” refers to a disease or disorder in which the noradrenergic neurons are effected, involved and/or implicated. By way of example and not limitation such diseases or disorders include Parkinson's disease, schizophrenia, drug addition or anxiety disorder.

As used herein, the term “modulate” refers to an alteration or modification in the function and/or activity of a dopaminergic and/or noradrenergic neuron. By way of example, such alteration or modification may include, but is not limited to, enhancement or diminishment of activity and/or function and/or and/or survival, enhancement and/or diminishment of symptoms associated with a dopaminergic and/or noradrenergic neuron activity and/or an amelerioation, mitigation of a disease or disorder and/or symptoms associated with a dopaminergic and/or noradrenergic neurons. Modulate is also intended to encompass enhancement or diminishment of polynucleotide drug target expression and/or polypeptide drug target expression in a dopaminergic and/or noradrenergic neurons

A “primer” is a short polynucleotide, generally with a free 3′—OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as DNA polymerase, and typically a thermally-stable polymerase enzyme. Methods for PCR are well known in the art, and taught, for example in “PCR: A PRACTICAL APPROACH” (M. MacPherson et al., IRL Press at Oxford University Press (1991)). All processes of producing replicate copies of a polynucleotide, such as PCR or gene cloning, are collectively referred to herein a “replication.” A primer can also be used as a probe in hybridization reactions, such as Southern or Northern blot analyses. Sambrook et al., supra.

Reference to a Figure or Table herein is used to refer to any individual polynucleotide drug target listed in that Figure or Table or combinations of the polynucleotide drug targets listed in the Table or Figure. When more than one Figure or Table is referenced herein, reference is to any individual polynucleotide drug target in the referenced Figures or Tables or combinations of the polynucleotide drug targets from any one or more of the Figures or Tables. Likewise, reference to a Figure or Table herein is used to refer to any individual polypeptide drug target encoded by any individual polynucleotide drug target listed in that Figure or Table or combinations of polypeptide drug targets encoded by the polynucleotide drug targets listed in the Table or Figure. When more than one Figure or Table is referenced herein, reference is to any individual polypeptide drug target encoded by any individual polynucleotide drug target in the referenced Figures or Tables or combinations of the polypeptide drug targets encoded by the polynucleotide drug targets from any one or more of the Figures or Tables.

The invention provides methods of identifying polynucleotide drug targets and/or polypeptide drug targets in dopaminergic and/or noradrenergic neurons, methods of screening for agents that modulate dopaminergic neurons and/or noradrenergic neuron activity, function and/or polynucleotide and/or polypeptide drug target expression. This invention also provides the polynucleotide and/or polypeptide drug targets identified by the methods described herein and to kits for use in the methods described herein. This invention is based on a discovery that anatomically and functionally distinct populations of dopaminergic neurons and/or noradrenergic neurons express distinct polynucleotides drug targets (e.g., gene expression profiles). As used herein, “drug target(s)” generally refers to polynucleotides and/or polypeptides identified by the methods described herein.

### Methods of identifying Drug Targets

In one aspect this invention provides a method of identifying candidate drug targets in a dopaminergic and/or noradrenergic neuron comprising: (a) identifying and/or isolating a population of dopaminergic and/or noradrenergic neurons; (b) evaluating the expression of one or more polynucleotides in the population of dopaminergic and/or noradrenergic neurons, wherein the the one or more polynucleotides and/or the one or more encoded polypeptides are candidate drug targets. The method may further comprise evaluating the expression of one or more polynucleotides in step (b) relative to a control population of neurons (e.g., whole brain, a population of neurons other than that being screened for drug targets).

In another aspect this invention relates to a method of identifying candidate drug targets in a population of dopaminergic or noradrenergic neurons comprising evaluating the expression of one or more polynucleotides in a dopaminergic or noradrenergic neuron population, wherein the one or more polynucleotides and their corresponding polypeptides are candidate drug targets.

Any population of dopaminergic and/or noradrenergic neurons may be used in the methods described herein. The population of dopaminergic and/or noradrenergic neurons may be obtained from a variety of sources and or samples. Examples include, but are not limited to mammals such as humans, primates or rodents (e.g., rats, mice), pathology, autopsy or biopsy samples, brain tissue banks, or in vitro cultures of dopaminergic and/or noradrenergic neurons. By way of example, but not limitation, midbrain dopaminergic neurons or noradrenergic neurons from the locus coeruleus can be used. The selection of the particular population of dopaminergic and/or noradrenergic neurons to be used in the method will, in part be directed by the particular dopaminergic and/or noradrenergic neuron disease or disorder for which the drug target is being sought.

Generally, for evaluating the polynucleotide or gene expression profile of a population of dopaminergic neurons and/or noradrenergic neurons, the population of neurons must be identified and/or isolated from the other cells in the starting sample. The selected population of dopaminergic neurons and/or noradrenergic neurons can be identified by a variety of morphological and/or molecular criteria (e.g., anatomical location and/or known gene expression in conjunction with in situ or immunocytochemistry or pigmentation in human and primates). By way of example, dopaminergic and noradrenergic neurons can be identified by tyrosine hydroxolase immunostaining or, in primates or humans, by neuromelanin pigmentation. Subpopulations of dopaminergic neurons in the substania nigra that differ in their susceptibility to degeneration in Parkinson's disease can be selected by their anatomical location (e.g., ventral or dorsal part of the substania nigra) or expression of vulnerability factors such as, for example, calbindin, capase-3 and/or glutamate receptors. Once identified the dopaminergic and/or noradrenergic neurons may be isolated by methods known in the art, including, but not limited to, laser microdissection (e.g., PALM Microlaser Technology).

Once the one or more neuronal cells of the dopaminergic and/or noradrenergic population are identified and/or isolated, the gene profile or gene expression cells can be evaluated by methods known in the art. Examples include, but are not limited to, PCR, microarray analysis in conjunction with RT-PCR, in situ or immunohistochemistry. By way of example, in situ hybridization in combination with a microarray can be utilized. Generally, the expression of one or more polynucleotides or gene expression profile of the dopaminergic and/or noradrenergic neurons is evaluated relative to the polynucleotide expression pattern of a control, such as, for example, whole brain tissue or a different population of neurons. Parameters for selecting candidate drug targets include, but are not limited to, polynucleotides and/or polypeptides specifically expressed in the dopaminergic and/or noradrenergic neurons relative to a control. Specifically expressed is intended to include, but is not limited to expression in the populations of the dopaminergic and/or noradrenergic neurons relative to absence of expression in the control or enhanced or diminished expression in the populations of the dopaminergic and/or noradrenergic neurons relative to the control. Statistical algorithms or commercially available statistical programs can be used to determine if the expression is statistically significant. By way of example, between about five to about eight fold and above difference in expression may be used to identify drug targets.

In a preferred embodiment, the method of the invention utilizes immunostaining and laser microdissection for identification and/or isolation of the dopaminergic and/or noradrenergic neurons and in situ hybridization to evaluate the expression of the one or more polynucleotides. In this embodiment, the sample comprising the dopaminergic and/or noradrenergic neurons is sectioned and mounted on slides. Preferably, the sections are mounted on slides engineered for maximal laser cutting and catapulting efficiency. By way of example, a 1.35 μm polyethylene naphthalene membrane can be sealed to a slide with about 0.1% poly-L-lysine followed by UV irradiation for about 30 minutes. The slide with the membrane can be further treated with 0.1% poly-L-lysine for about 5 minutes and allowed to dry to further overcome the hydrophobic nature of the membrane and improve adherence of the tissue section the membrane/glass slide. The starting sample may be a dissected rat brain which or human brain sample, which was preferably immediately frozen on dry ice prior to use and/or stored at −80 C until sectioning. Frozen tissue is sectioned on the cryostat at, for example, about 12 micron thickness and on pre-processed polyethylene naphthalene membrane slides. Sections are fixed, preferably immediately in 100% ethanol for about 30 seconds followed by a brief dip in acetone (e.g., less than or about 2-3 seconds) and air-dried at room temperature. RNA quality is greatly enhanced if the section are rehydrated in phosphate buffered saline (PBS) at a pH of about 7.0 to about 7.5 (higher pH leads to increased degradation of RNA) containing 1 about 1 to about 2 U/ul RNAse inhibitor (e.g., from Roche, Germany) for about 5 seconds. If the target population of neurons is dopaminergic or noradrenergic neurons, immunohistochemical staining to is utilized to detect tyrosine hydroxlase. Briefly, sections are stained with 100 μg/ml labeled primary antibody in PBS pH7 containing 1 U/ul RNAse inhibitor (Roche, Germany) for 3 min. Tyrosine hydroxylase positive cells are detected with anti-tyrosine hydroxylase antibodies (e.g., clone TH-16, Sigma, USA). The antibody is purified with a protein A column and is covalently labeled with a fluorophore that has, for example, a succinimidlyl ester moiety that reacts with primary amines of proteins to form stable dye-protein conjugates. Kits are commercially for fluorophore labeling are commercially available, for example, the Alexa Fluor 488 monoclonal antibody labeling kit may be used following manufacturer's instructions. The labeled antibody is purified via gel filtration column chromatography followed by three washes in a buffered aqueous solution, such as PBS in a Microcon 30 centrifugal filter device. The sections are then washed in PBS (about, for example, pH 7.0) twice for 5 seconds, followed by dehydration for 30 seconds each in 75%, 95%, and 100% ethanol respectively and dried at room temperature.

Immunostained cells are dissected utilizing laser microdissection dissection (Schutze K and Lahr G. (1998) Nat. Biotechnol 16(8);737-742). By way of example, with a PALM Robot-Microbeam system (PALM microlaser technology, Germany) may be used. To facilitate detection of fluorescent cells, generally a drop of 100% ethanol is applied to the section while the cells are selected. Sections are allowed to air dry at room temperature for about 5 minutes and the cells, by way of example about 200 cells, are dissected and catapulted into about 30 μl of lysis buffer.

RNA may be isolated by conventional methodology. In a preferred embodiment total RNA is isolated by silica-gel spin columns after homogenization of the cells in a denaturing guanidine isothiocyanate containing buffer. By way of example, a commercial kit such as the Picopure kit (Arcturus) may be used. In a preferred embodiment the RNA is amplified using T7-based linear amplification. By way of example, the RNA is amplified by two rounds of T7-based linear amplification (Van Gelder et al., (1990) Proc. Natl. Acad. Sci (USA) 87:1663-1667). Briefly, mRNA is converted into cDNA using an oligo-dT primer that contains a T7 RNA polymerase promoter site. The double-stranded CDNA is used as template for T7 RNA polymerase to transcribe antisense RNA which is amplified up to 1000 fold compared to the original input MRNA. The antisense RNA is used for a second round of amplification resulting in about 10⁶-fold amplification. For amplification, by way of example, the Riboamp kit (Arcturus) was used according to the manufacturer's protocol. The reaction can be enhanced by the following modifications, to avoid generation of template-independent amplification product from the T7 primer, a five fold dilution of primer A was used for first round cDNA synthesis and the reaction volume was scaled down by 50%.

The amplification products can be characterized by a variety of methods known in the art. Nonlimiting examples include, assessment of the amplification product by microfluidic gel electrophoresis with, for example, with the Agilent bioanalyzer, hybridization. Products that show the expected amount and size distribution of RNA molecules (about 200 to about 2000 nucleotides) can be hybridized to DNA microarrays.

Polynucleotides

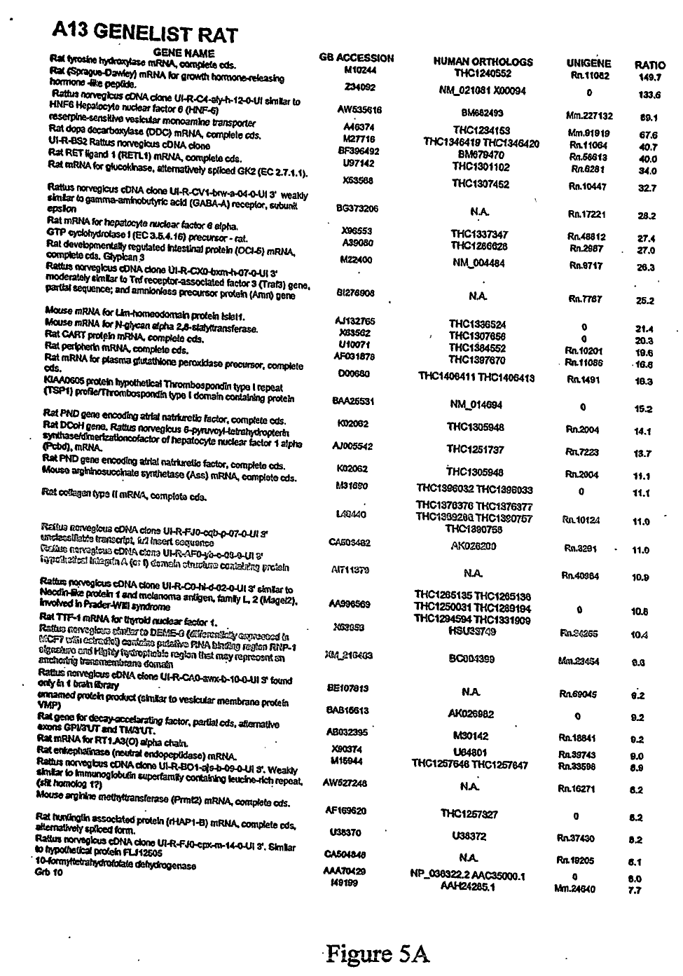

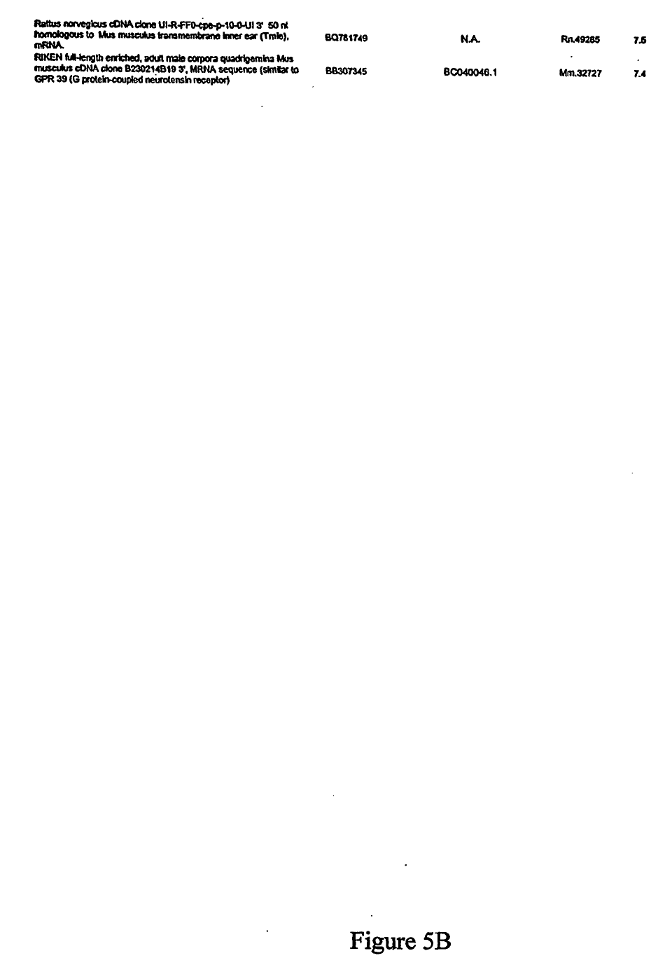

Another aspect of this invention is directed to isolated polynucleotide drug targets identified by the method described herein. The term polynucleotide is used broadly and refers to polymeric nucleotides of any length (e.g., oligonucleotides, genes, small inhibiting RNA, fragments of polynucleotides encoding a protein etc). By way of example, the polynucleotides of the invention may comprise the coding sequence for the active or functional domains of a protein or the intact protein and or non-coding sequences (e.g., regulatory sequences, introns etc). The polynucleotide of the invention may be, for example, linear, circular, supercoiled, single stranded, double stranded, branched, partially double stranded or single stranded. The nucleotides comprising the polynucleotide may be naturally occurring nucleotides or modified nucleotides. The polynucleotides referenced in FIGS. 5-15 and Tables 1-4, and/or their complements represent preferred embodiments of the invention. It is, however, understood by one skilled in the art that due to the degeneracy of the genetic code variations in the polynucleotide sequences shown will still result in a polynucleotide sequence capable of encoding a drug target. Such polynucleotide sequences are therefore functionally equivalent to the sequence set forth in FIGS. 5-15 and Tables 1-4 and are intended to be encompassed within the present invention. Further, a person of skill in the art will understand that there are naturally occurring allelic variations of the polynucleotide sequences shown in FIGS. 5-15 and Tables 1-4 are also intended to be encompassed by the present invention

This invention also relates to homologs or orthologs of the polynucleotide sequences referenced in FIGS. 5-15 and Tables 1-4 and/or their complements. The homologs or orthologs may be identified by methods known in the art. A variety of sequence alignment software programs are available in the art to facilitate determination of homology or equivalence. Non-limiting examples of these programs are BLAST family programs including BLASTN, BLASTP, BLASTX, TBLASTN, and TBLASTX (BLAST is available from the worldwide web at ncbi.nln.nih.gov/BLAST/), FastA, Compare, DotPlot, BestFit, GAP, FrameAlign, ClustalW, and PileUp. These programs are obtained commercially available in a comprehensive package of sequence analysis software such as GCG Inc.'s Wisconsin Package. Other similar analysis and alignment programs can be purchased from various providers such as DNA Star's MegAlign, or the alignment programs in GeneJockey. Alternatively, sequence analysis and alignment programs can be accessed through the world wide web at sites such as the CMS Molecular Biology Resource at sdsc.edu/ResTools/cmshp.html. Any sequence database that contains DNA or protein sequences corresponding to a gene or a segment thereof can be used for sequence analysis. Commonly employed databases include but are not limited to GenBank, EMBL, DDBJ, PDB, SWISS-PROT, EST, STS, GSS, and HTGS.

Parameters for determining the extent of homology set forth by one or more of the aforementioned alignment programs are well established in the art. They include but are not limited top value, percent sequence identity and the percent sequence similarity. P value is the probability that the alignment is produced by chance. For a single alignment, the p value can be calculated according to Karlin et al. (1990) Proc. Natl. Acad. Sci. (USA) 87: 2246. For multiple alignments, the p value can be calculated using a heuristic approach such as the one programmed in BLAST. Percent sequence identify is defined by the ratio of the number of nucleotide or amino acid matches between the query sequence and the known sequence when the two are optimally aligned. The percent sequence similarity is calculated in the same way as percent identity except one scores amino acids that are different but similar as positive when calculating the percent similarity. Thus, conservative changes that occur frequently without altering function, such as a change from one basic amino acid to another or a change from one hydrophobic amino acid to another are scored as if they were identical.

By way of example, polynucleotides of the invention are about 60%, more preferably greater than about 70%, even more preferably greater than about 80% and most preferably greater than 90% (e.g., 93% or 95% or 98%) identity to one of the polynucleotide sequences referenced in FIGS. 5-15 and Tables 1-4 and/or their complements.

This invention also relates to a polynucleotide that hybridizes under stringent conditions to a polynucleotide referenced in FIGS. 5-15 and Tables 1-4. Hybridization reactions can be performed under conditions of different “stringency”. Conditions that increase stringency of a hybridization reaction of widely known and published in the art. See, for example, Sambrook et al. (2001). Examples of relevant conditions include (in order of increasing stringency): incubation temperatures of 25° C., 37° C., 50° C. and 68° C.; buffer concentrations of 10×SSC, 6×SSC, 4×SSC, 1×SSC, 0.1×SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or deionized water. In a preferred embodiment hybridization and wash conditions are done at high stringency. By way of example hybridization may be performed at 50% formamide and 4×SSC followed by washes of 2×SSC/formamide at 50° C. and with 1×SSC.

Polypeptides

Another aspect of this invention is directed to isolated polypeptide drug targets identified by the methods described herein. The term polypeptide is used broadly herein to include peptide or protein or fragments thereof. Examples of fragments include, but are not limited to fragments comprising the active or functional domains of a protein. Also intended to be encompassed are peptidomimetics, which include chemically modified peptides, peptide-like molecules containing nonnaturally occurring amino acids, peptoids and the like, have the selective binding of the targeting domains provided herein. (“Burger's Medicinal Chemistry and Drug Discovery” 5th ed., vols. 1 to 3 (ed. M. E. Wolff; Wiley Interscience 1995).

This invention further includes polypeptides or analogs thereof having substantially the same function as the polypeptides of this invention. Such polypeptides include, but are not limited to, a substitution, addition or deletion mutant of the polypeptide. This invention also encompasses proteins or peptides that are substantially homologous to the polypeptides.

A variety of sequence alignment software programs described herein above are available in the art to facilitate determination of homology or equivalence of any protein to a protein of the invention.

The term “analog” includes any polypeptide having an amino acid residue sequence substantially identical to at least one of the polypeptide sequences encoded by the polynucleotides referenced in FIGS. 5-15 and Tables 1-4 in which one or more residues have been conservatively substituted with a functionally similar residue and which displays the functional aspects of the polypeptides as described herein. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid or another.

The phrase “conservative substitution” also includes the use of a chemically derivatized residue in place of a non-derivatized residue. “Chemical derivative” refers to a subject polypeptide having one or more residues chemically derivatized by reaction of a functional side group. Examples of such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those proteins or peptides which contain one or more naturally-occurring amino acid derivatives of the twenty standard amino acids. For examples: 4-hydroxyproline may be substituted for proline; 5-hydroxylysine may be substituted for lysine; 3-methylhistidine may be substituted for histidine; homoserine may be substituted for serine; and ornithine may be substituted for lysine. Polypeptides of the present invention also include any polypeptide having one or more additions and/or deletions or residues relative to the sequence of a any one of the polypeptides whose sequences is described herein.

By way of example, polypeptides of the invention are at least about 60%, 65%, 70%, 75%, 80%, 80%, 85%, 90%, 95%, 96%, 97%, 98%, or 99% identical to any polypeptide encoded by a polynucleotide sequence referenced in FIGS. 5-15 and Tables 1-4. In some embodiments, the polypeptide is at least about 70% or 80% or 90% or 95% identical to any polypeptide encoded by a polynucleotide sequence referenced in FIGS. 5-15 and Tables 1-4.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

Preferably, the “percentage of sequence identity” is determined by comparing two optimally aligned sequences over a window of comparison of at least 20 positions, wherein the portion of the polypeptide sequence in the comparison window may comprise additions or deletions (i.e. gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical r amino acid residue occurs in both sequences to yield the number of matched positions, dividing the number of matched positions by the total number of positions in the reference sequence (i.e. the window size) and multiplying the results by 100 to yield the percentage of sequence identity.

Markers

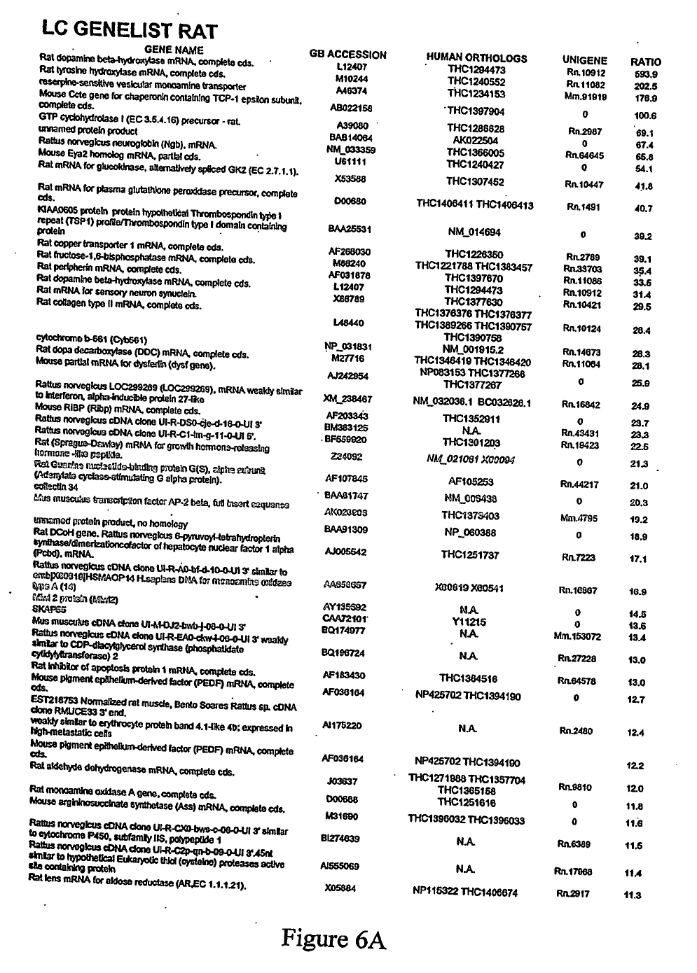

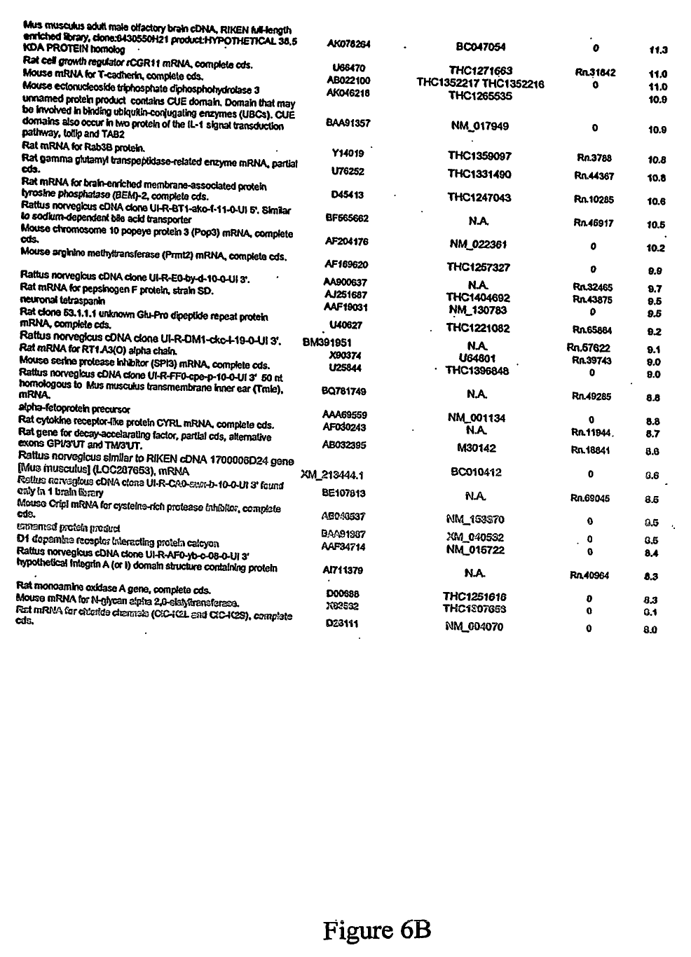

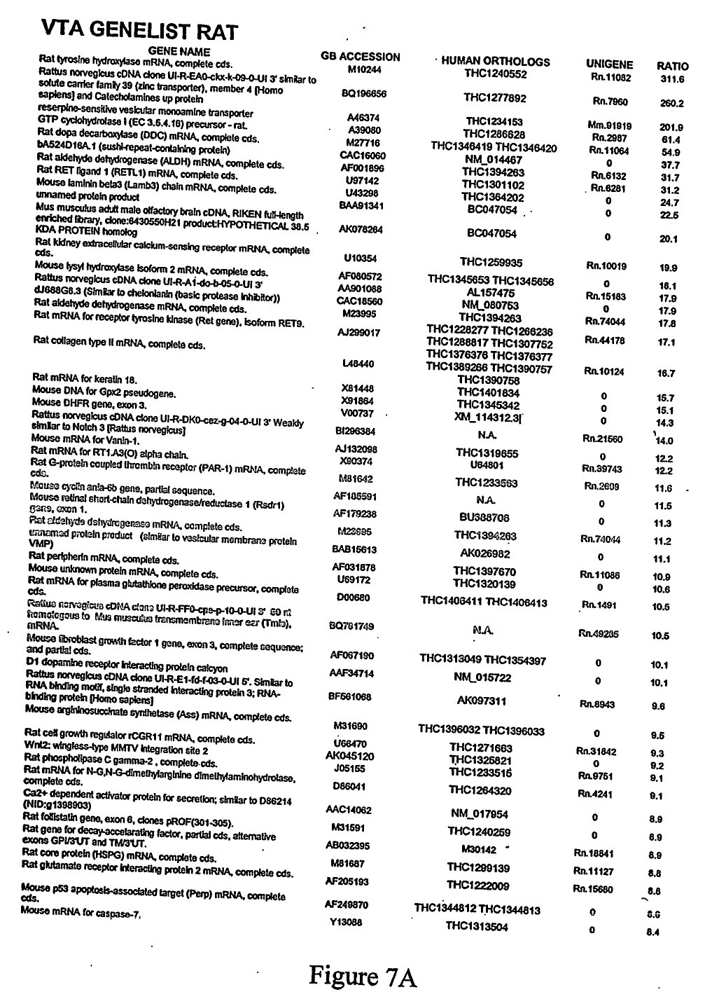

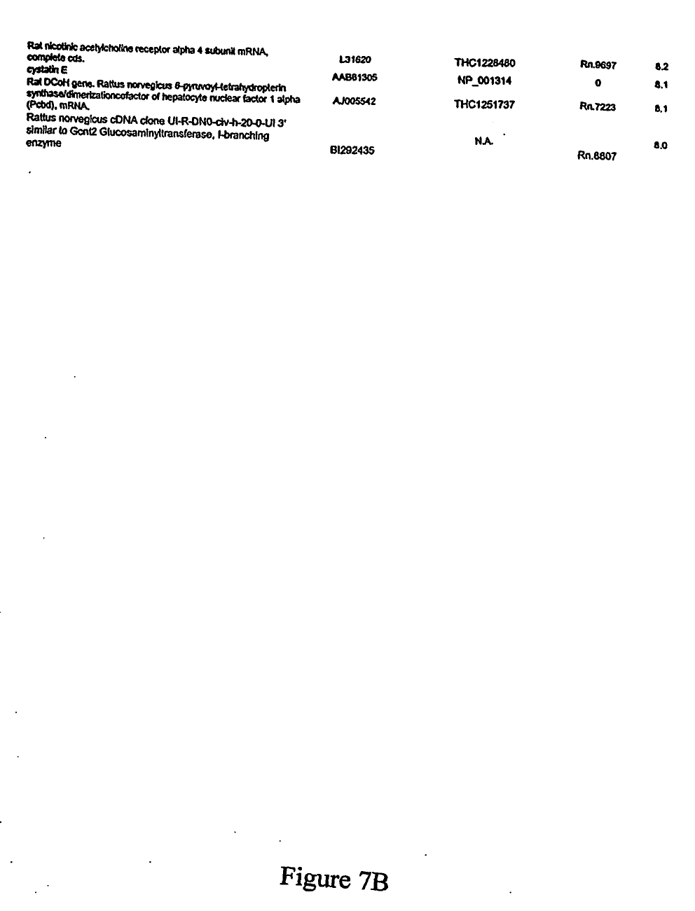

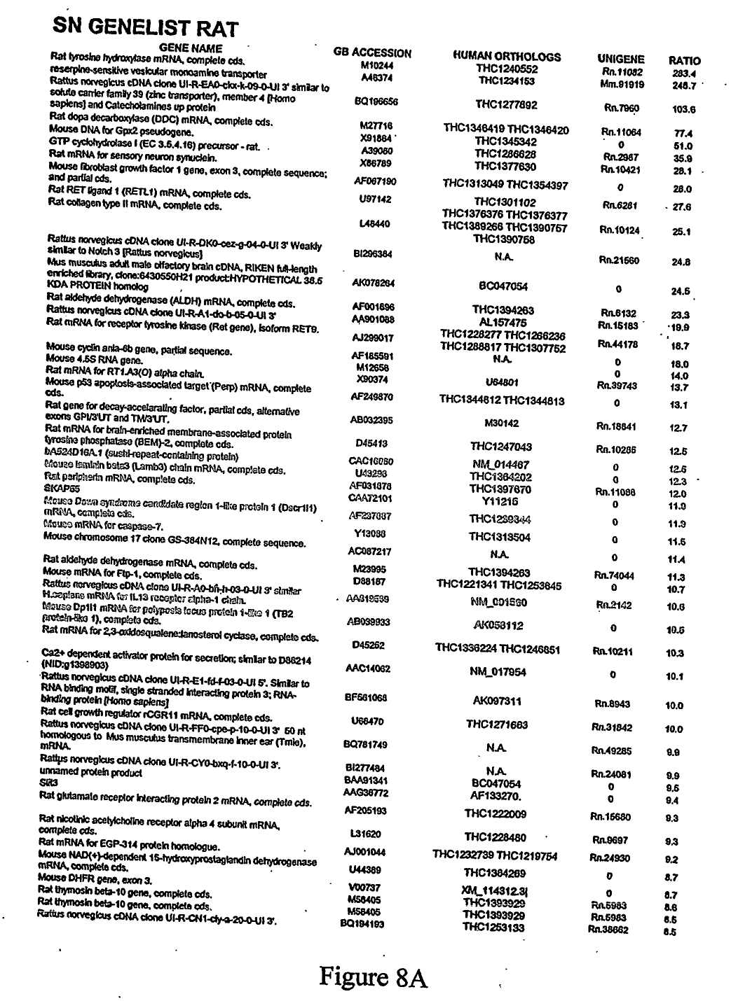

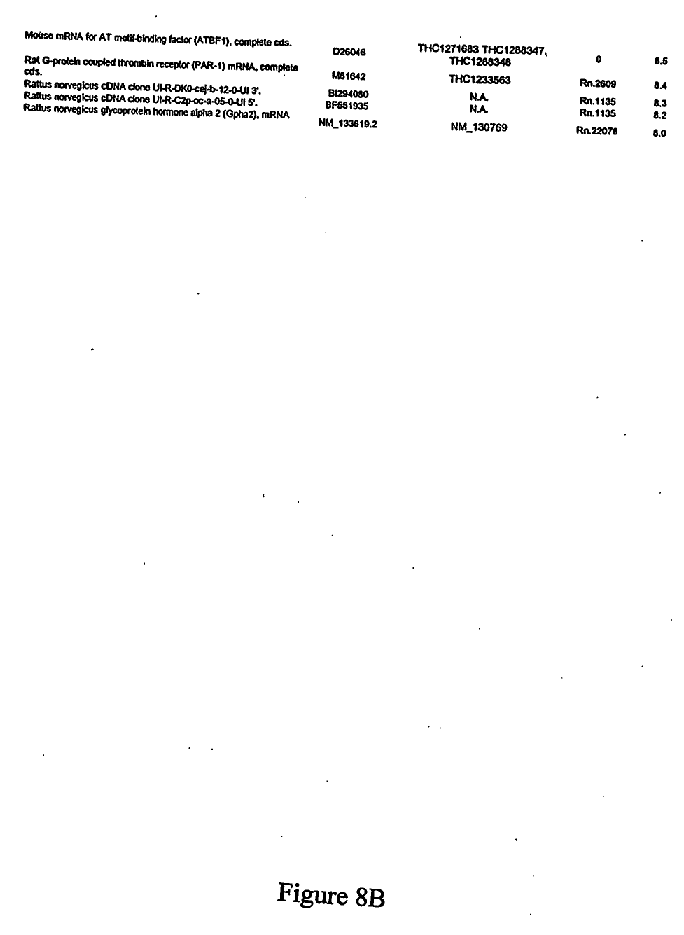

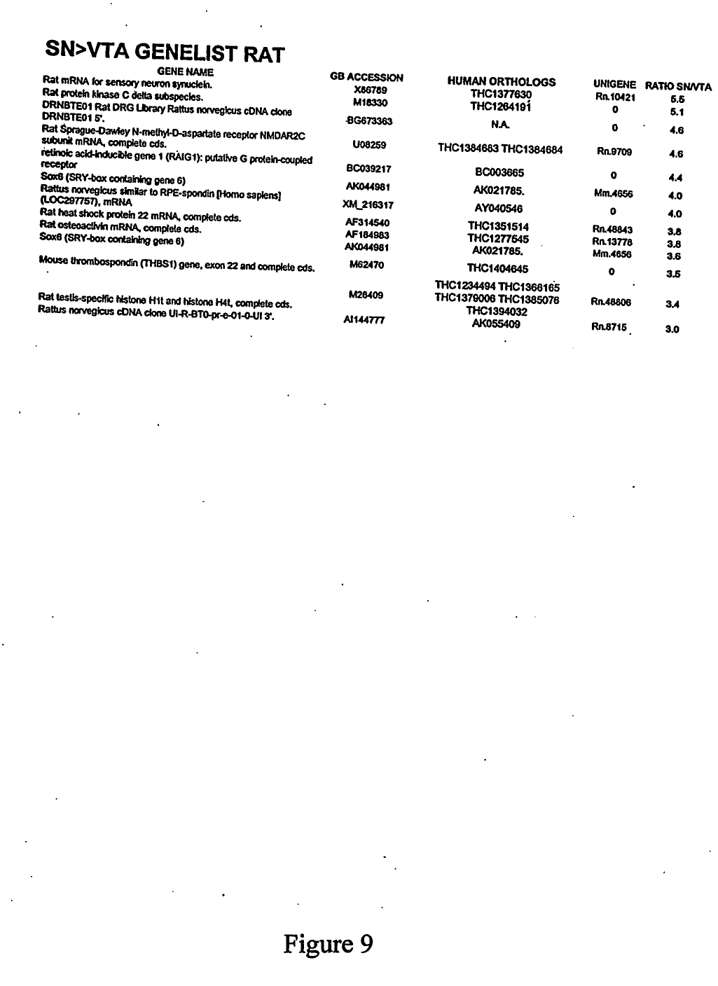

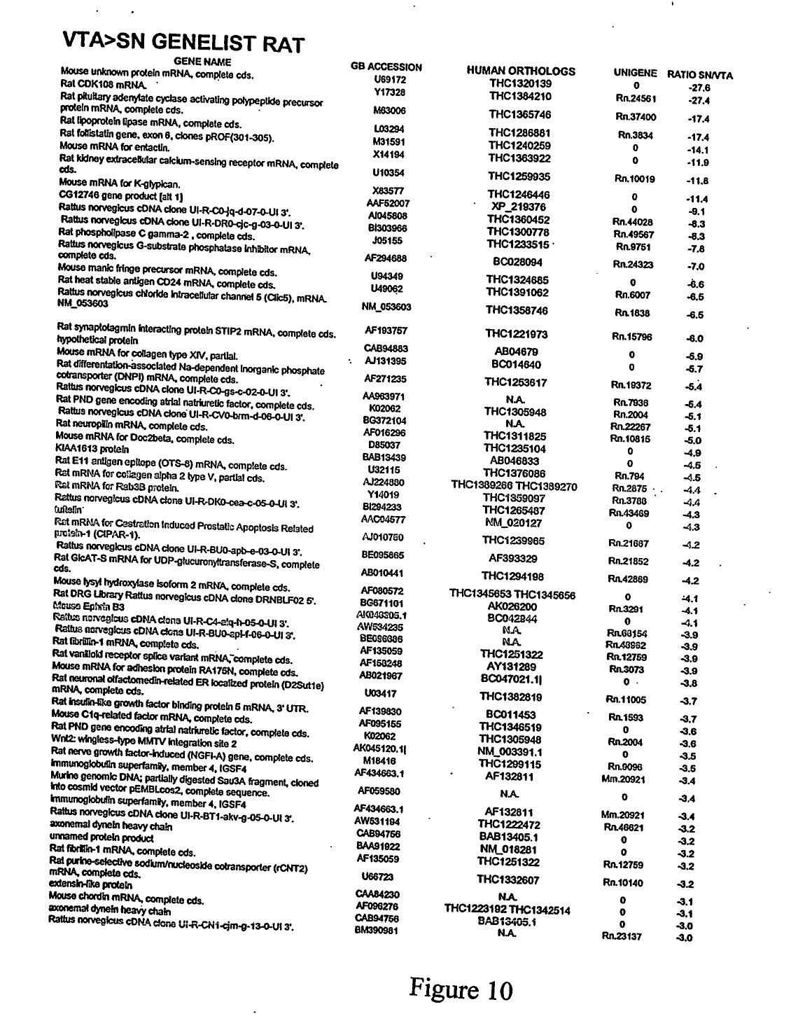

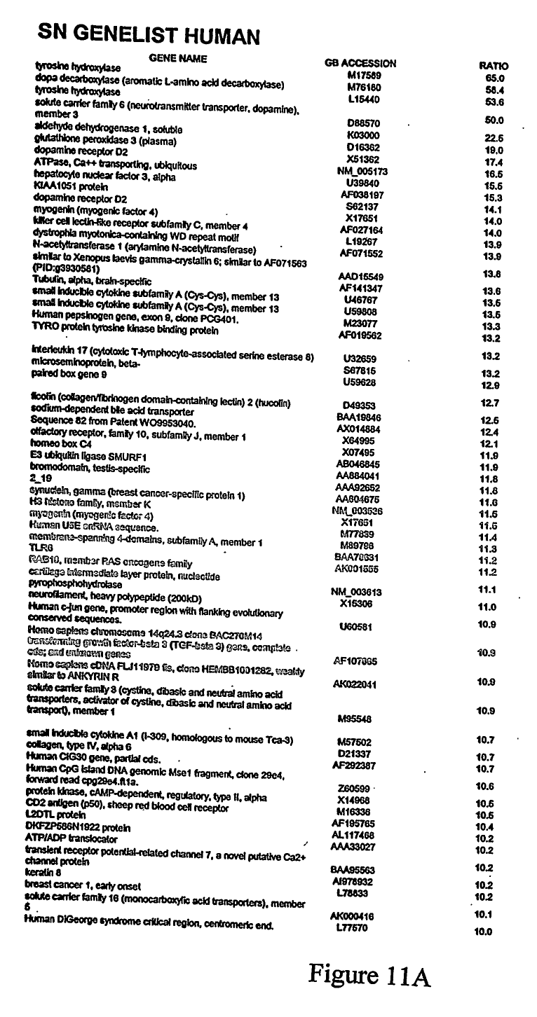

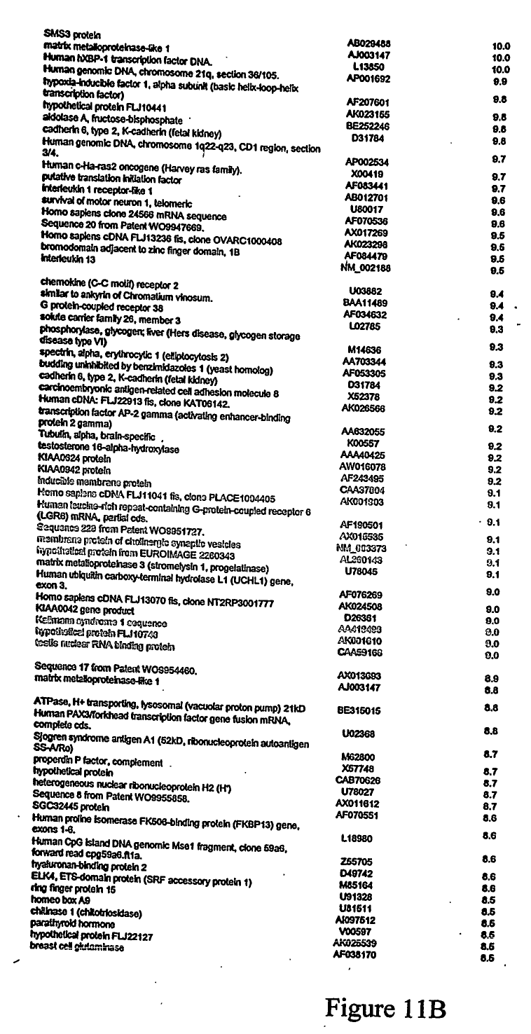

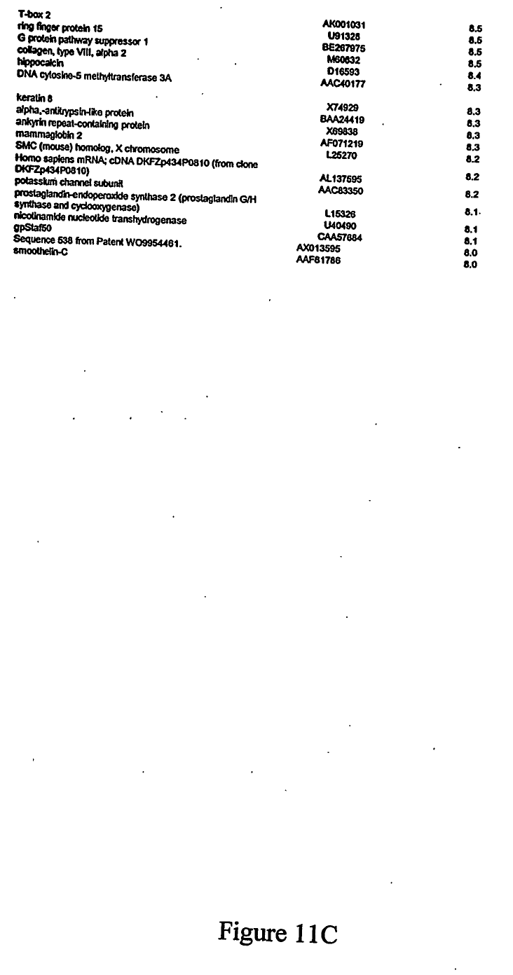

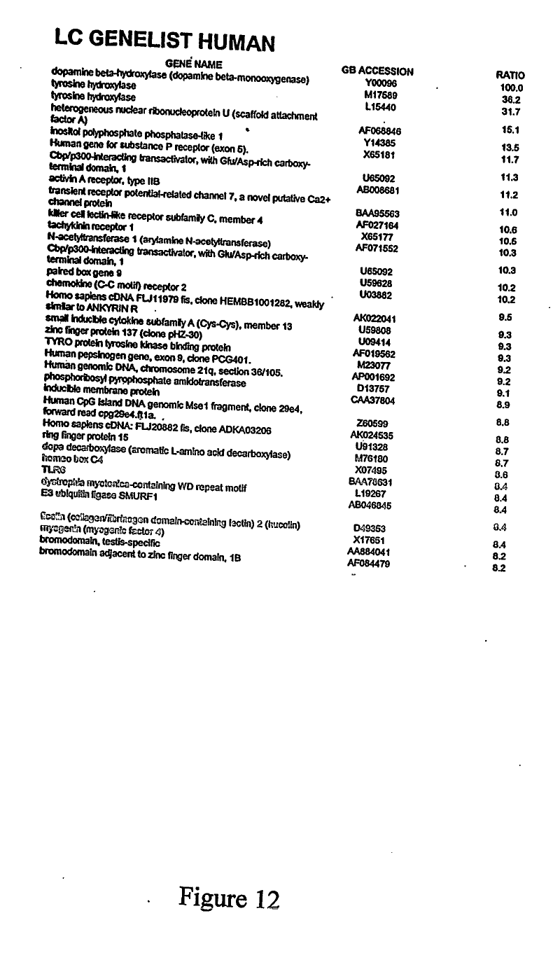

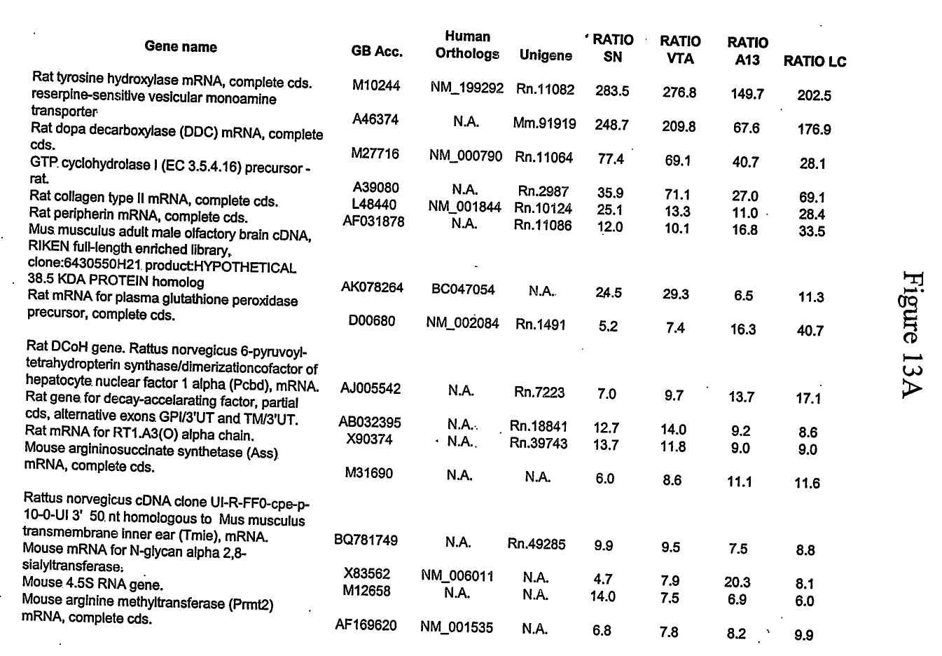

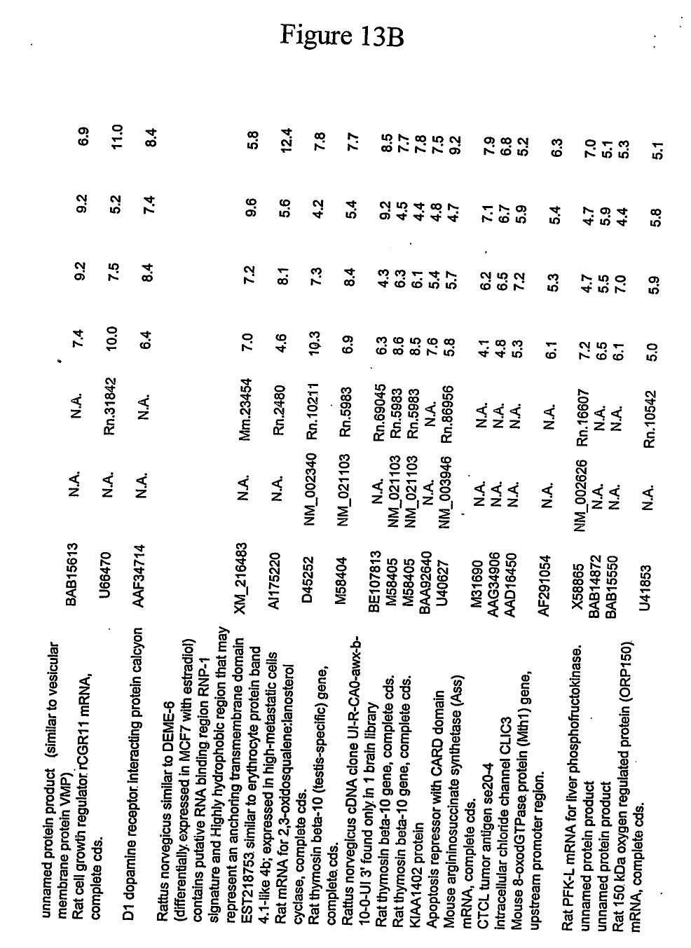

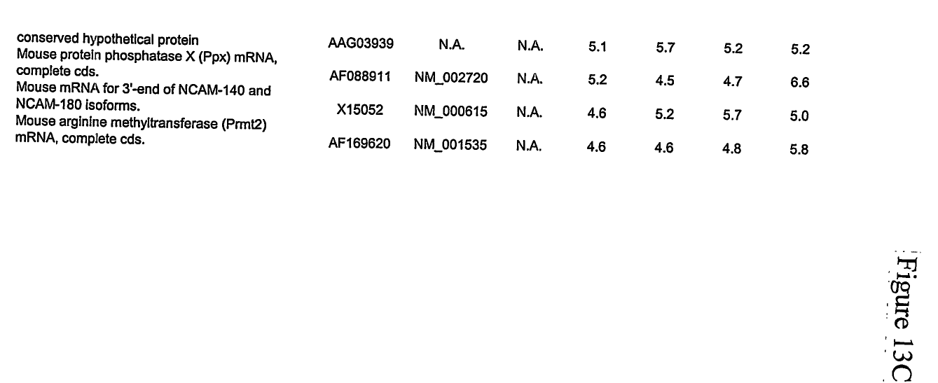

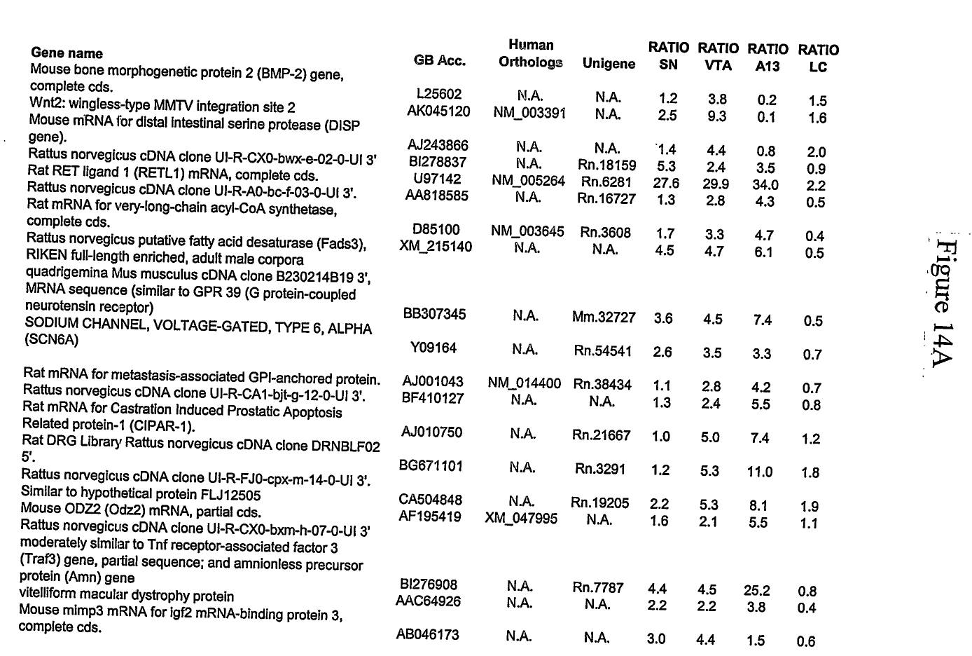

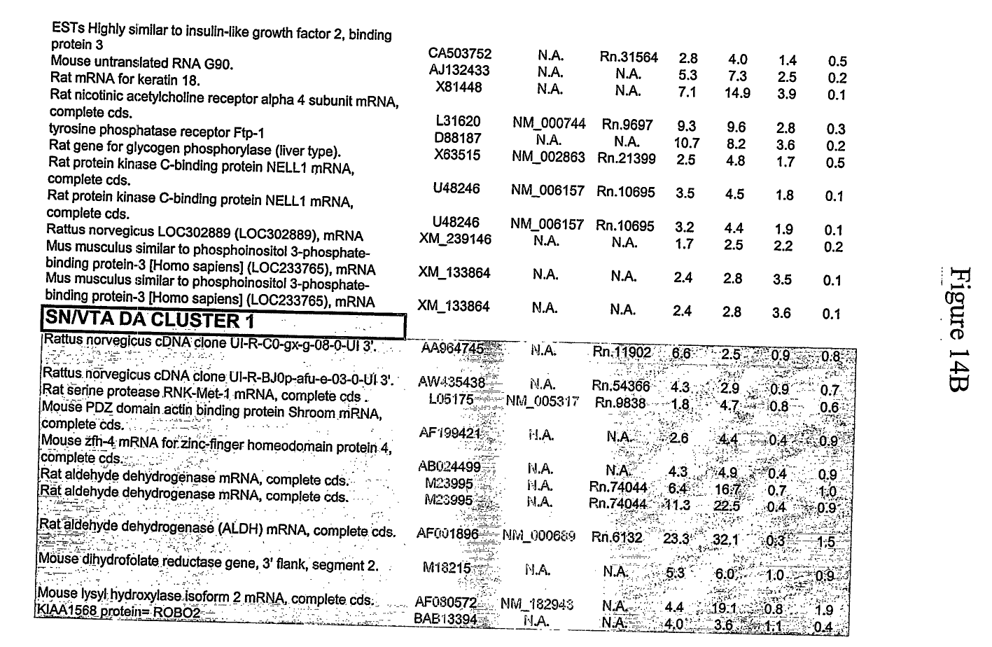

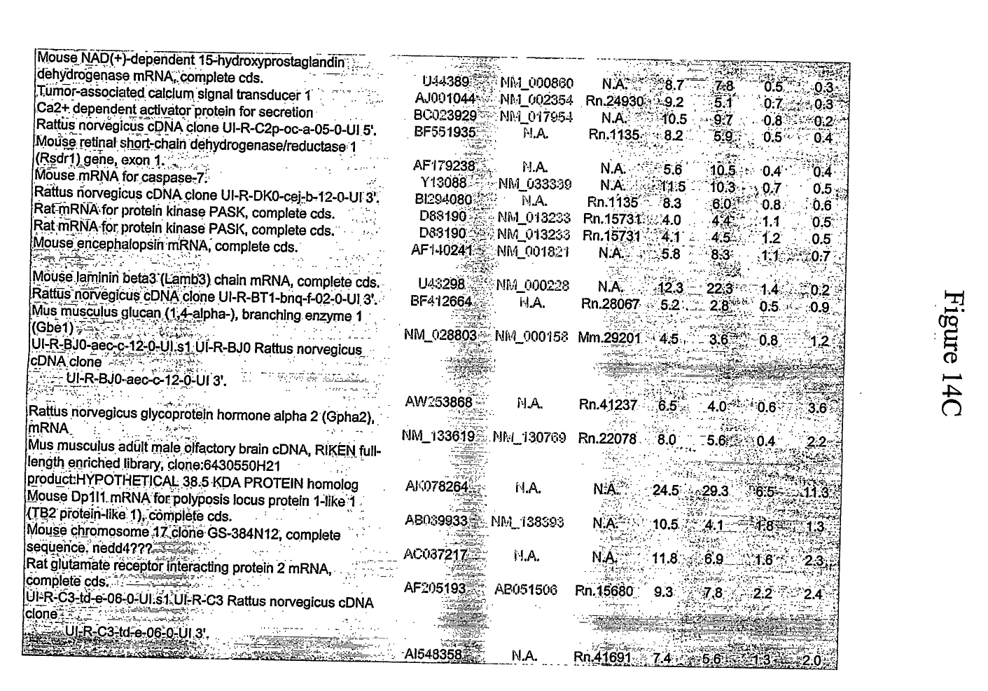

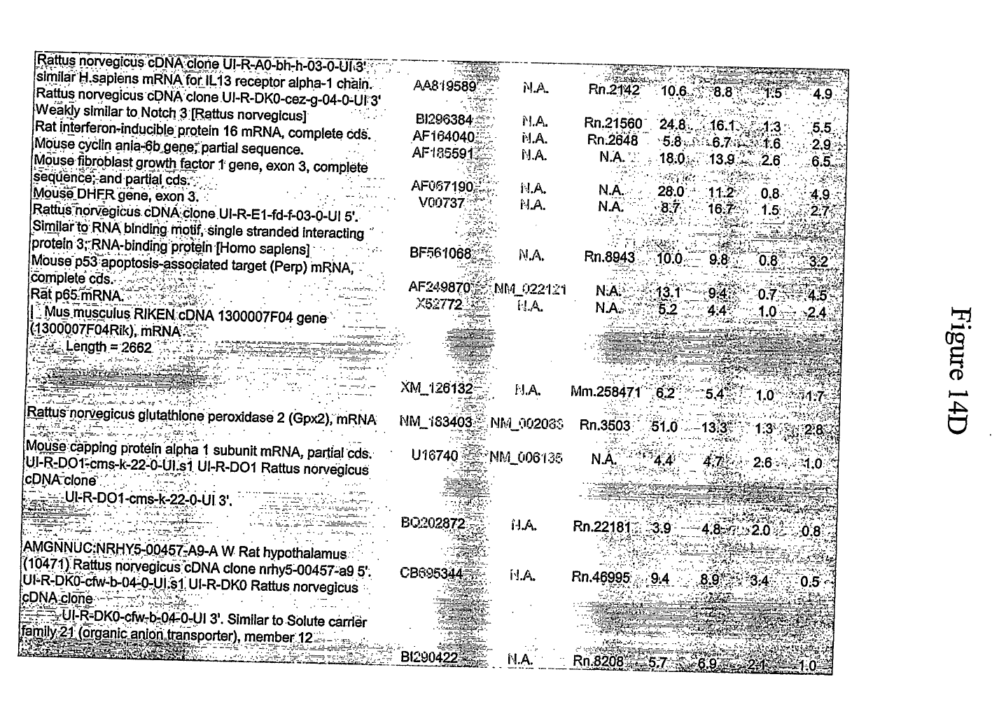

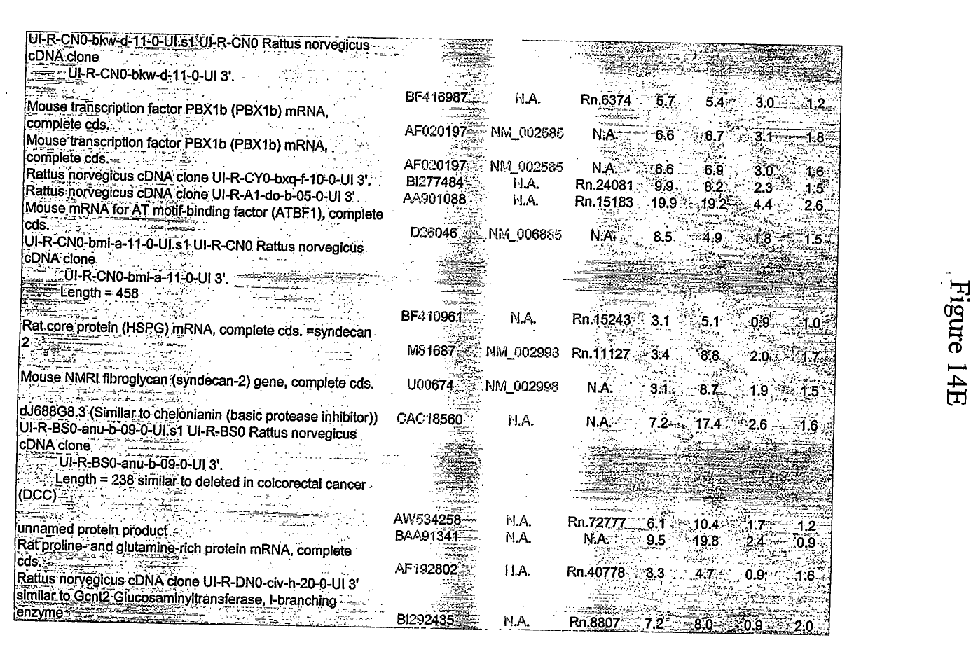

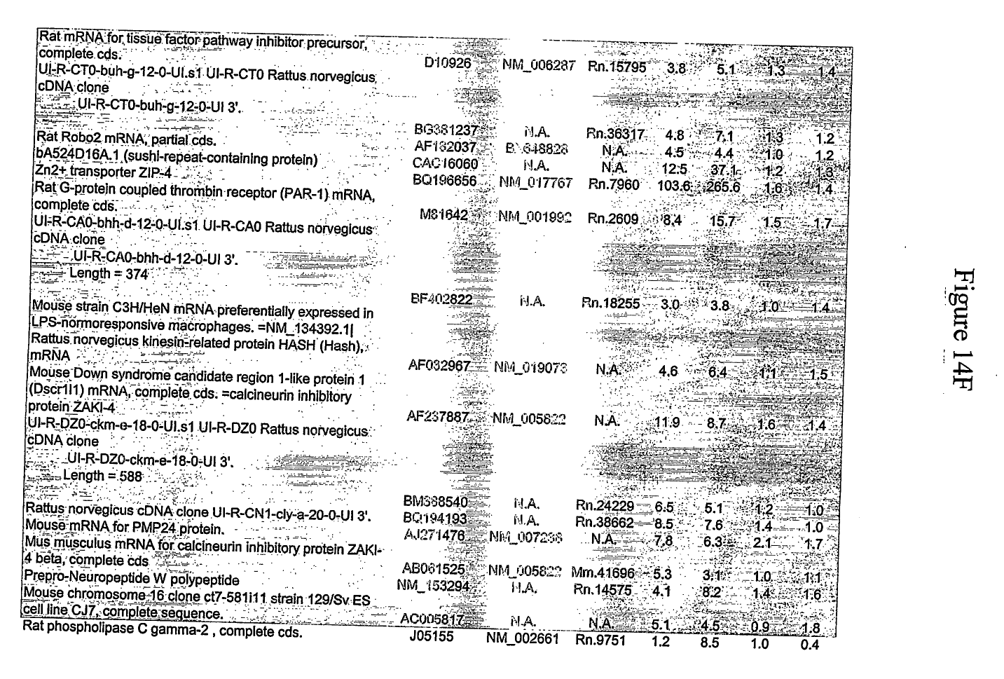

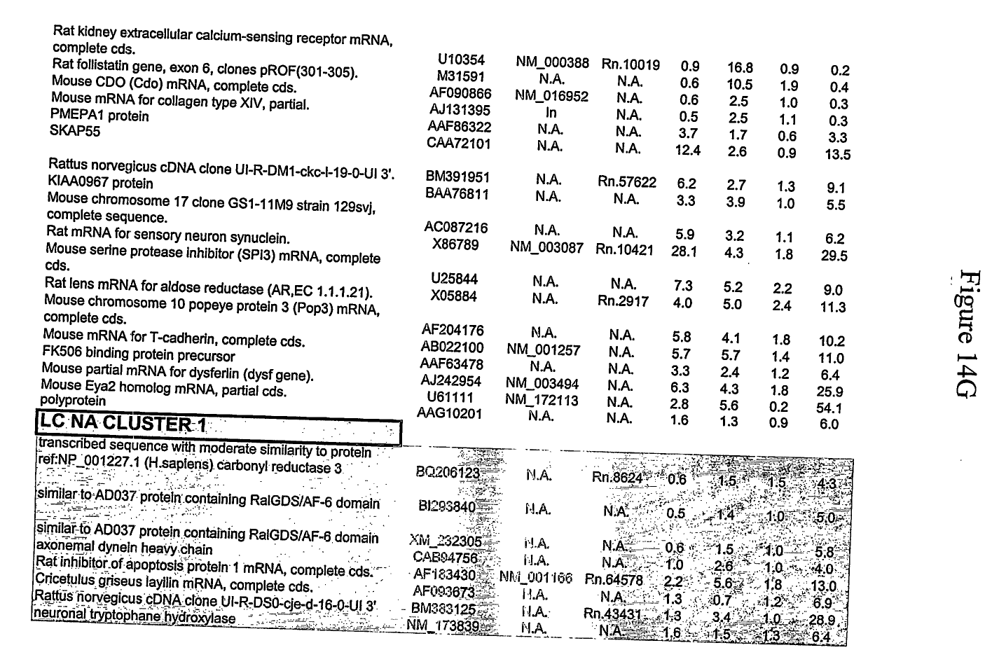

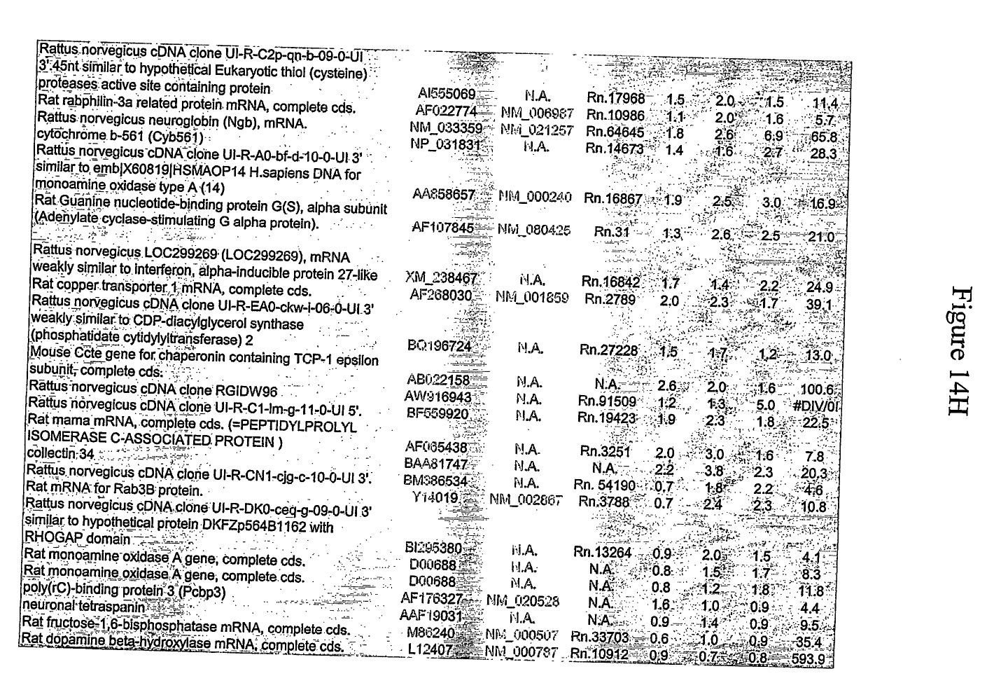

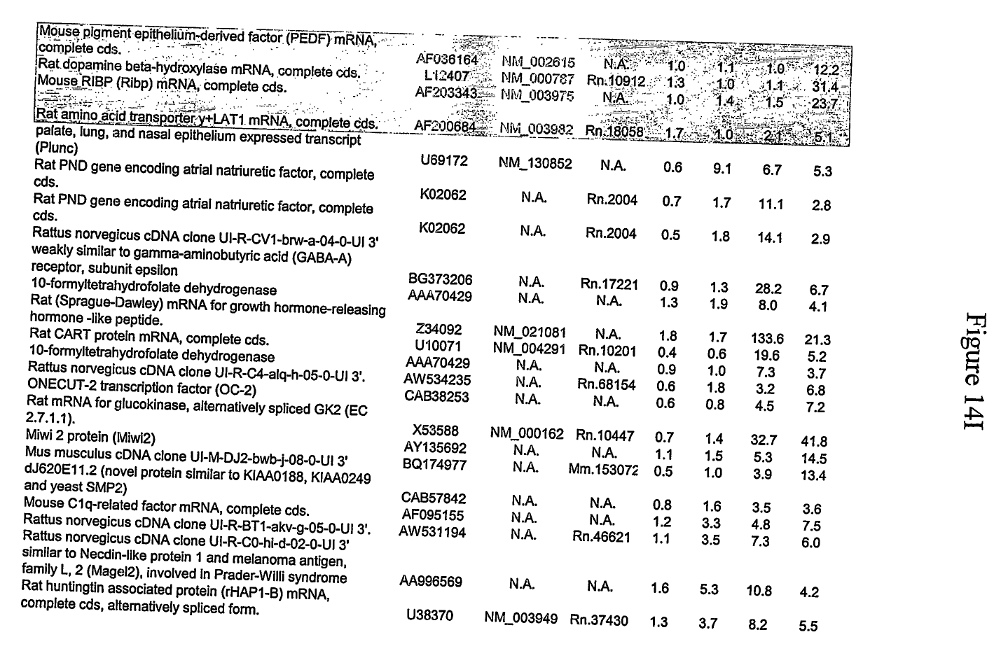

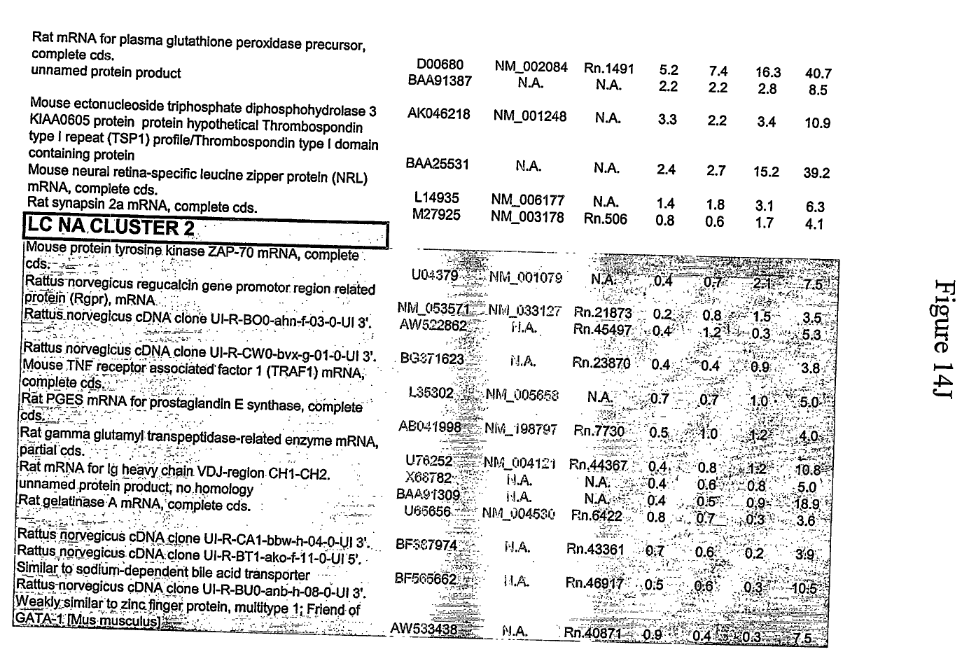

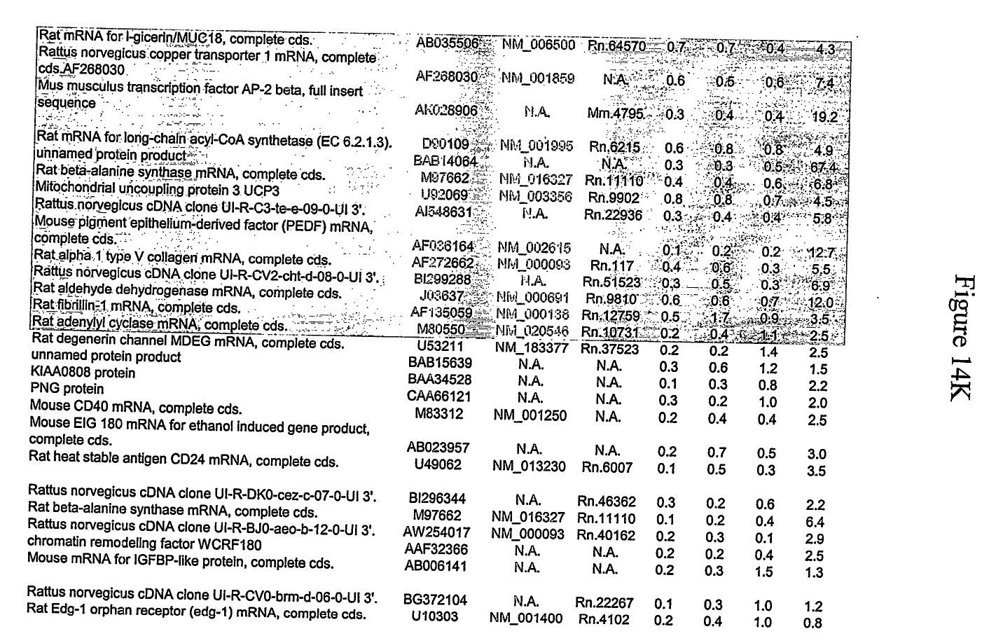

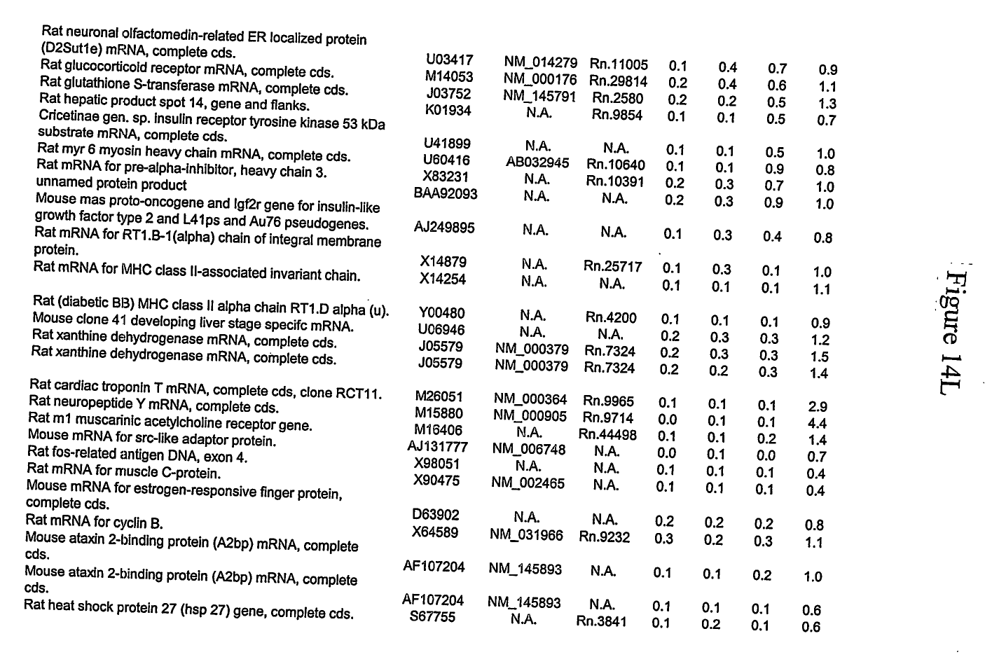

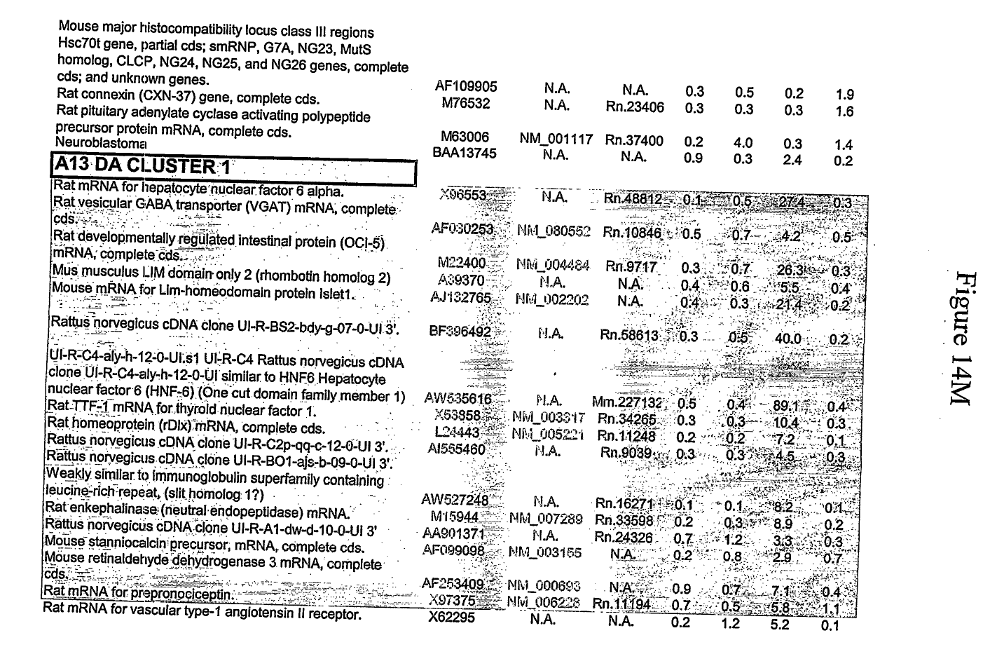

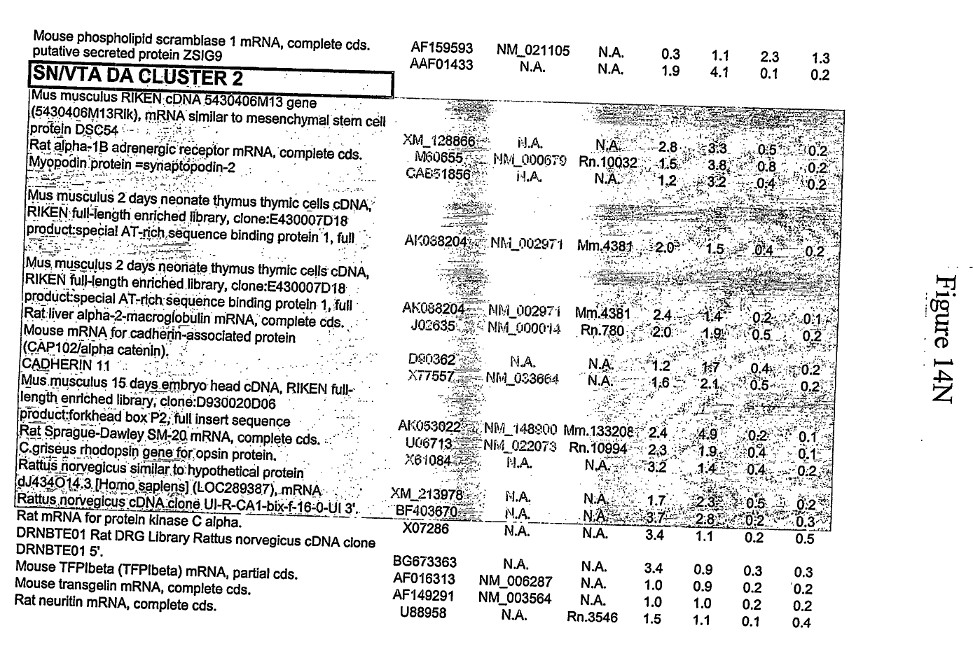

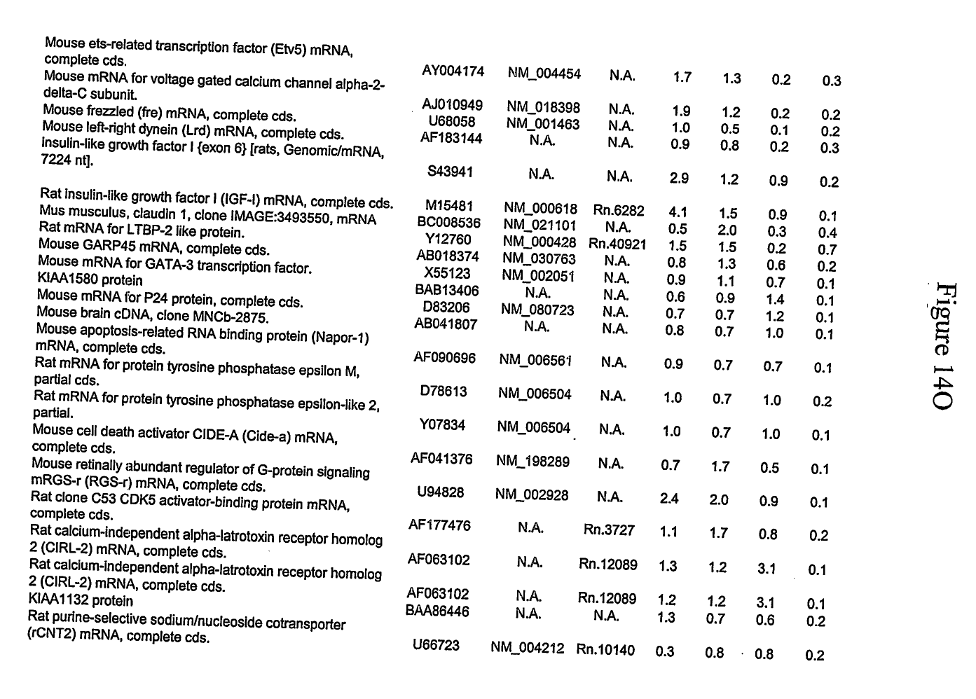

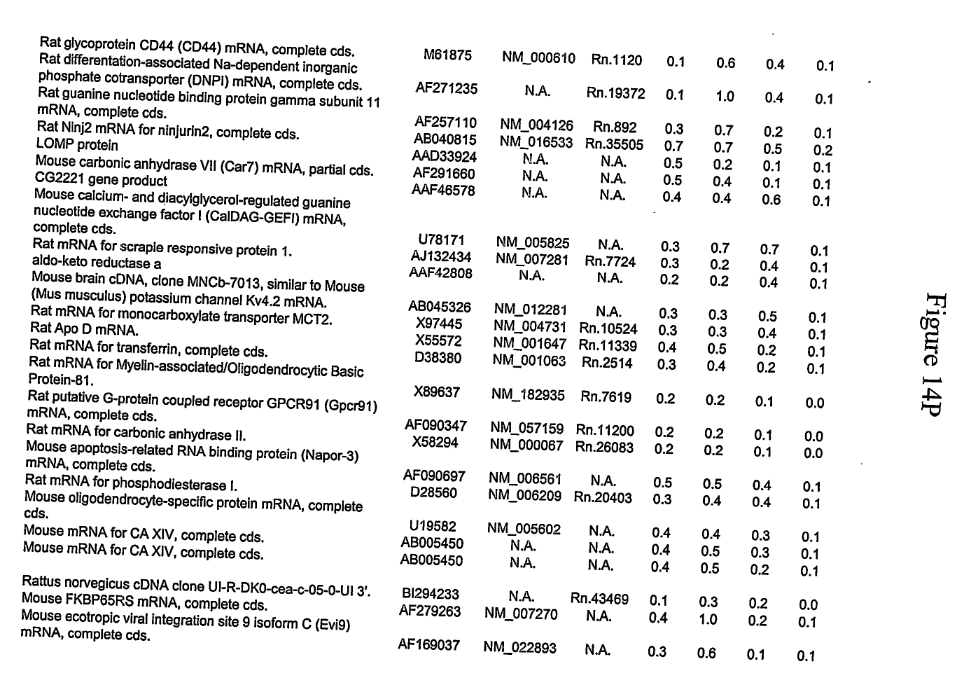

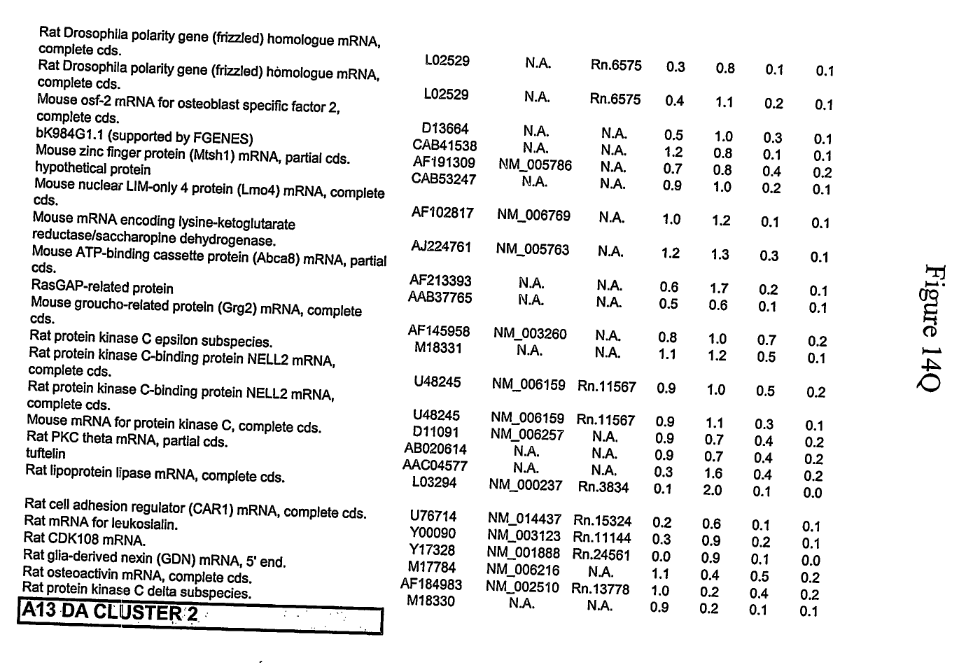

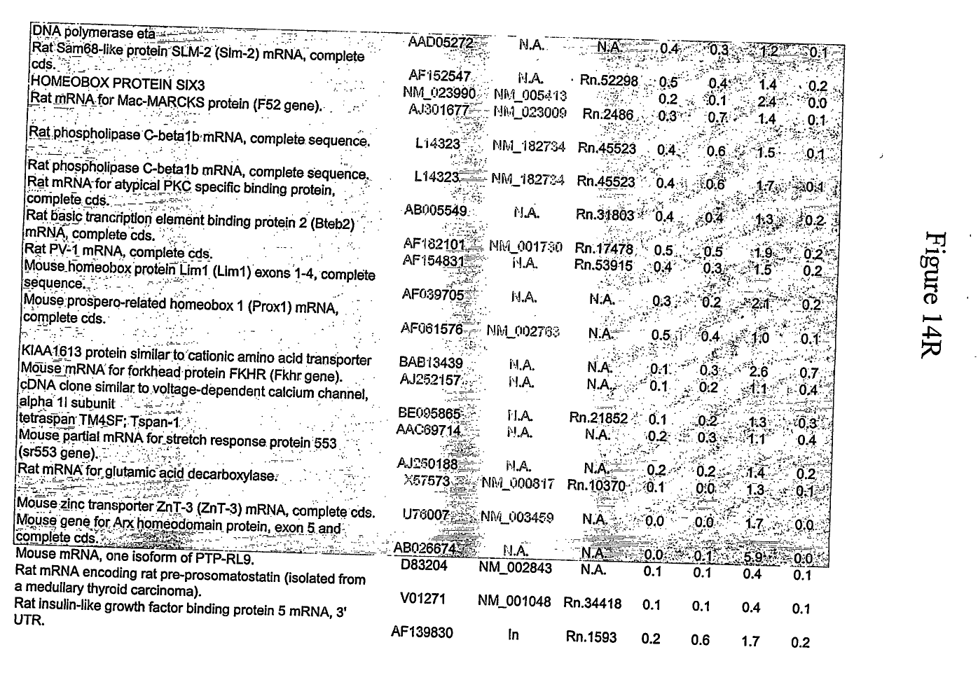

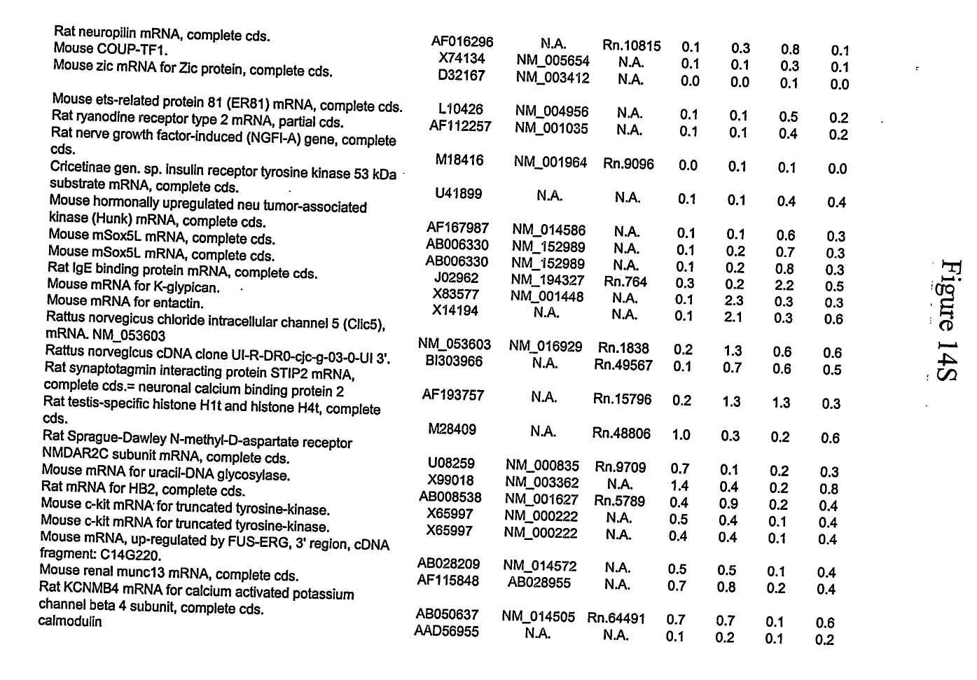

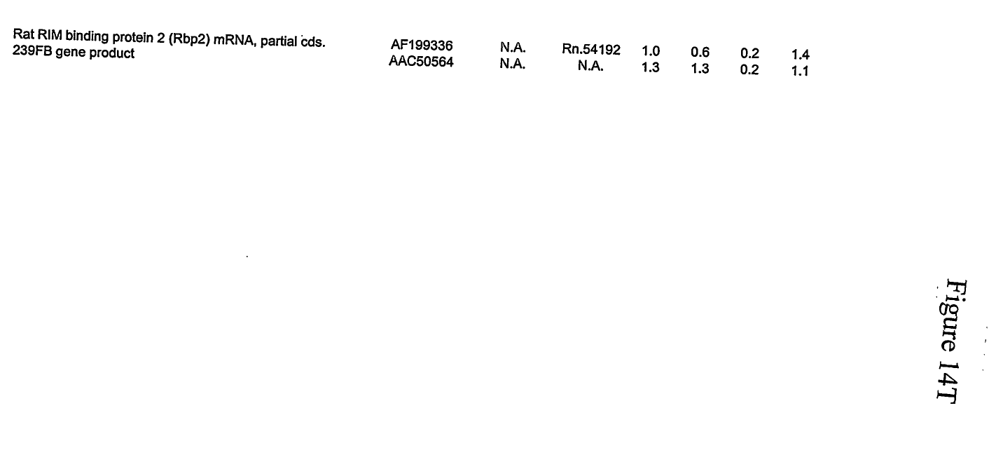

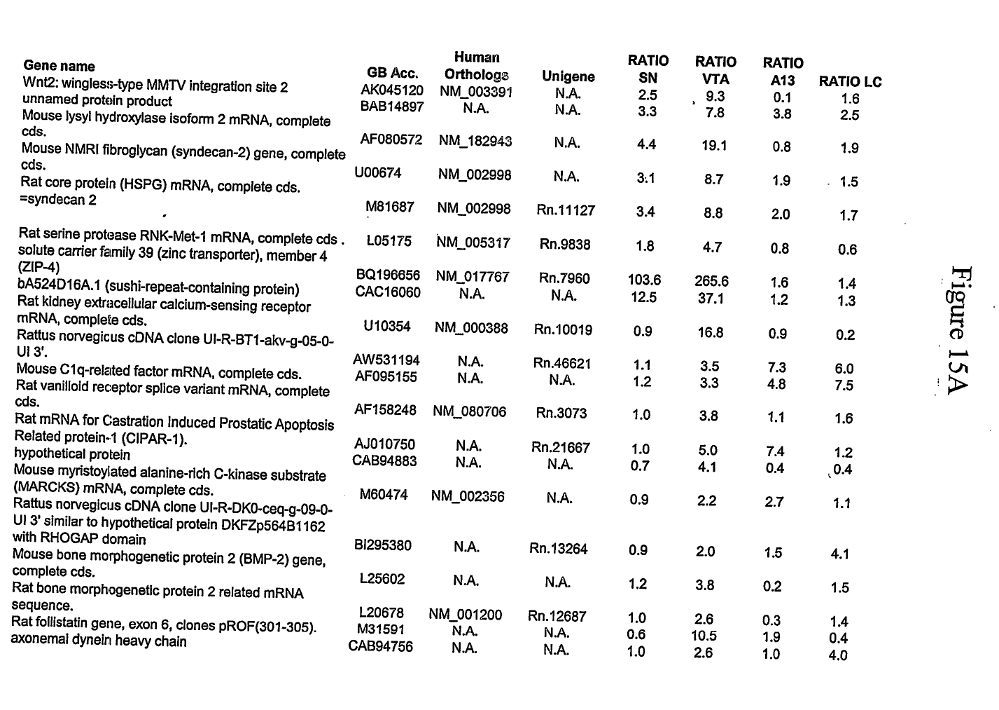

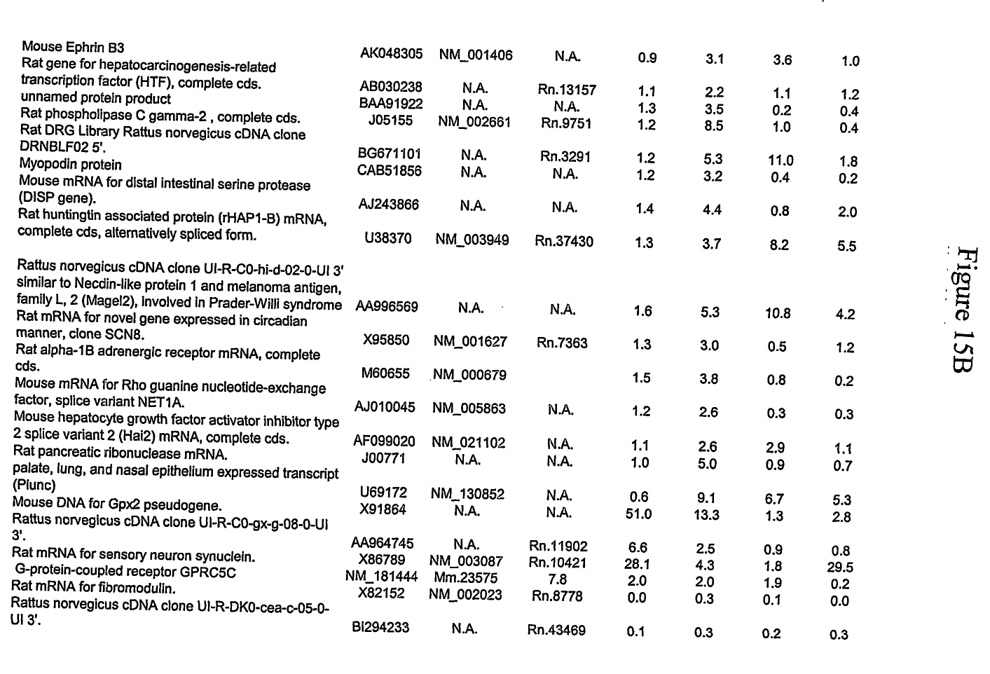

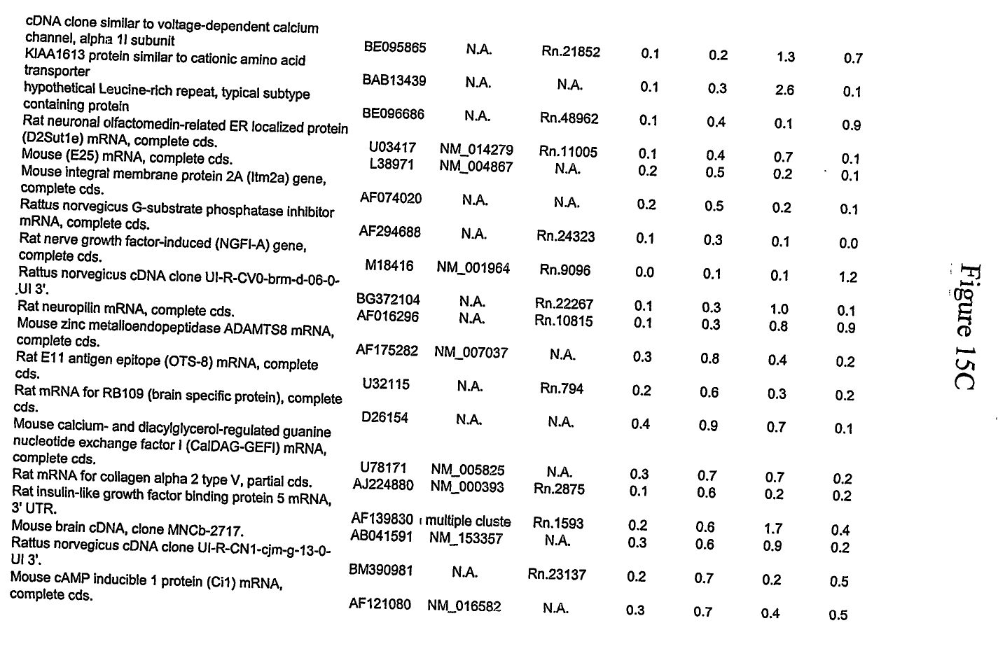

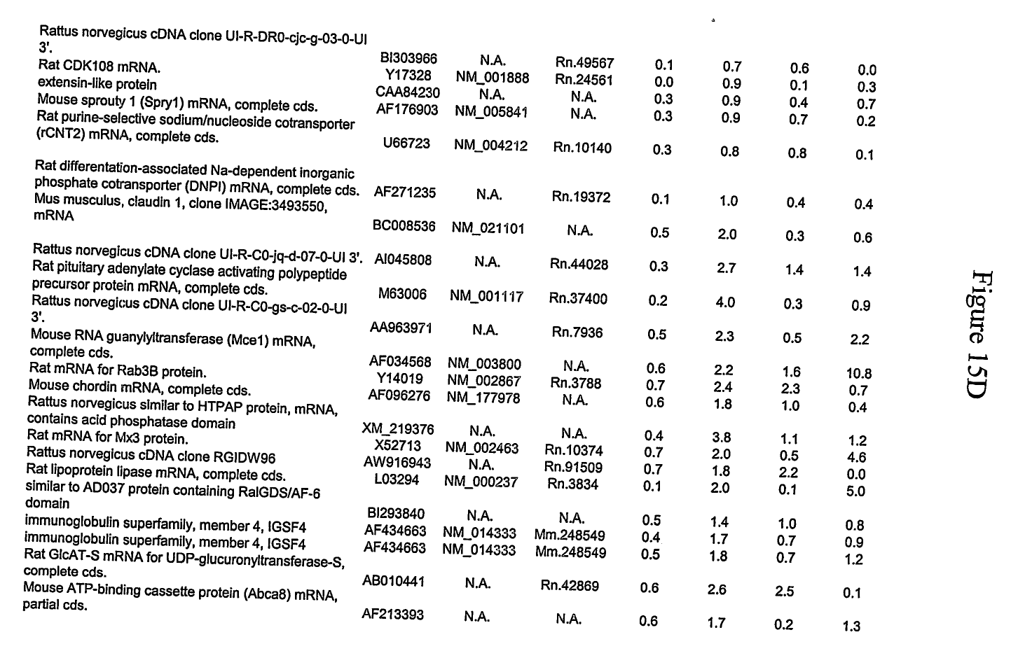

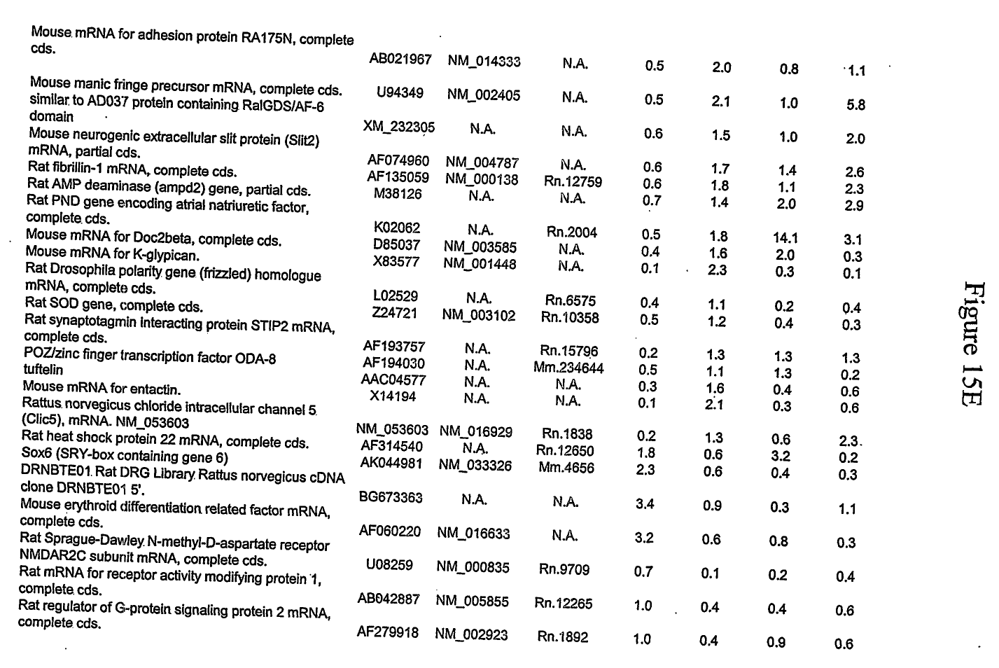

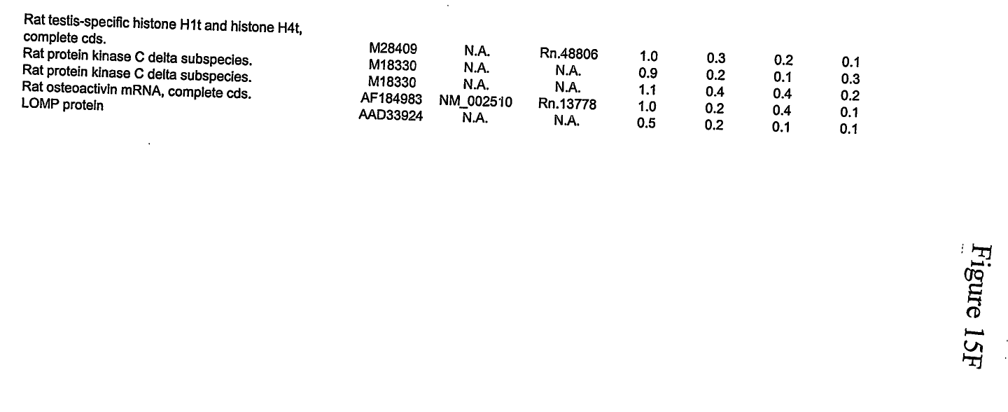

One or more of the polynucleotide drug targets and/or the polypeptide drug targets identified by the methods described herein can be used as markers to identify a population of neurons. For example, substania nigra dopaminergic cells may be identified by expression of one or more of the polynucleotide drug targets referenced in FIG. 8, FIG. 9, FIG. 10, FIG. 11, FIG. 13, FIG. 14, FIG. 15, Table 1, Table 2 and Table 4 and/or their corresponding polypeptides, zona incerta A13 neurons may be identified by expression of one or more of the polynucleotide drug targets referenced in FIG. 5, FIG. 13, FIG. 14 and Table 1 and/or their corresponding polypeptides, ventral tegmental area neurons may be identified by expression of one or more of the polynucleotide drug targets referenced in FIG. 7, FIG. 9, FIG. 10, FIG. 13, FIG. 14, FIG. 15, Table 1, Table 2 and Table 4 and/or their corresponding polypeptides, or neurons in the locus coeruleus may be identified by expression of the polynucleotide drug targets referenced in FIG. 6, FIG. 12, FIG. 13, FIG. 14, Table 1 and Table 3 and/or their corresponding polypeptides.

### Methods of Screening

This invention also provides for methods of screening for candidate agents that modulate the activity, function and/or expression profile of dopaminergic and/or noradrenergic neurons utilizing one or more polynucleotide drug targets and/or polypeptide drug target identified by the methods described herein. The screening assay may be performed either in vitro and/or in vivo. Candidate agents identified in the screening methods described herein may be useful as therapeutic agents for dopaminergic and /or noradrenergic diseases or disorders or one or more symptoms associated with dopaminergic and /or noradrenergic diseases or disorders. Examples of such diseases or disorders include, but are not limited to, Parkinson's disease, schizophrenia, drug addiction and anxiety disorders.

The one or more drug targets to be used in the screening method may be any polynucleotide drug target and/or polypeptides drug target identified by the methods described herein. The term polynucleotide is used broadly and refers to polymeric nucleotides of any length (e.g., oligonucleotides, genes, small inhibiting RNA, fragments of polynucleotides encoding a protein etc). By way of example, the polynucleotides of the invention may comprise the coding sequence for the active or functional domains of a protein or the intact protein and/or non-coding sequences. The polynucleotide of the invention may be, for example, linear, circular, supercoiled, single stranded, double stranded, branched, partially double stranded or single stranded. The nucleotides comprising the polynucleotide may be naturally occurring nucleotides or modified nucleotides. The polynucleotides referenced in FIGS. 5-15 and Tables 1-4 and/or their complement represent drug targets which may be used for screening. It is, however, understood by one skilled in the art that due to the degeneracy of the genetic code variations in polynucleotide sequences will still result in a polynucleotide sequence capable of encoding a drug target. Such polynucleotide sequences are therefore functionally equivalent to the sequence set forth in FIGS. 5-15 and Tables 1-4 and are intended to be encompassed within the present invention. Further, a person of skill in the art will understand that there are naturally occurring allelic variations of the polynucleotide sequences shown in FIGS. 5-15 and Tables 1-4 are also intended to be encompassed by the present invention. Additional examples of polynucleotides that may be used in the methods of screening for candidate agents include, but are not limited to, homologs or orthologs of the sequences referenced in FIGS. 5-15 and Tables 1-4 and polynucleotide that hybridizes under stringent conditions to a polynucleotide referenced in FIGS. 5-15 and Tables 1-4.

Likewise, one or more of the polypeptides identified as a drug target by the methods described herein may be utilized in the screening methods. The term polypeptide is used broadly herein to include peptide or protein or fragments thereof. Examples of fragments include, but are not limited to, fragments comprising the active or functional domains of a protein. By way of example, one or more of the polypeptides drug targets corresponding to the polynucleotide drug targets referenced in FIGS. 5-15 and Tables 1-4 may be used in the screening methods. Also intended to be encompassed are peptidomimetics of the polypeptides corresponding to the polynucleotides referenced in FIGS. 5-15 and Tables 1-4, polypeptides or analogs thereof having substantially the same function as the polypeptides corresponding to the polynucleotides referenced in FIGS. 5-15 and Tables 1-4 and polypeptides that are substantially homologous to the polypeptides corresponding to the polynucleotides referenced in FIGS. 5-15 and Tables 1-4.

The choice of the one or more drug targets will generally be directed by the population of neurons being screened. By way of example, for substania nigra dopaminergic cells one or more of the polynucleotide drug targets referenced in FIG. 8, FIG. 9, FIG. 10, FIG. 11, FIG. 13, FIG. 14, FIG. 15, Table 1, Table 2 and Table 4 and/or the polypeptides corresponding to the polynucleotides may be utilized, for zona incerta A13 neurons one or more of the polynucleotide drug targets referenced in FIG. 5, FIG. 13, FIG. 14 and Table 1 and/or the polypeptides corresponding to the polynucleotides may be utilized, for ventral tegmental area neurons one or more of the polynucleotide drug targets referenced in FIG. 7, FIG. 9, FIG. 10, FIG. 13, FIG. 14, FIG. 15, Table 1, Table 2 and Table 4 and/or the polypeptides corresponding to the polynucleotides may be utilized, and for neurons in the locus coeruleus one or more the polynucleotide drug targets referenced in FIG. 6, FIG. 12, FIG. 13, FIG. 14, Table 1 and Table 3 and/or the polypeptides corresponding to the polynucleotides may be utilized.

In one embodiment the method of assessing the ability of a candidate agent to modulate dopaminergic and/or noradrenergic neuron activity and/or function comprises: (a) contacting a population of dopaminergic and/or noradrenergic neurons expressing one or more polynucleotide and/or polypeptide drug targets with a candidate agent and (b) measuring the level of expression of the one or more polynucleotide and/or polypeptide drug targets in the population of dopaminergic and/or noradrenergic neurons, wherein an alteration of the level of expression of the one or more drug targets indicates the ability of a candidate agent to modulate dopaminergic and/or noradrenergic neuron activity and/or function and/or possible therapeutic potential of the candidate agent for treating one or more diseases or disorders associated with dopaminergic and/or noradrenergic neuron activity or one or more symptoms associated with dopaminergic and/or noradrenergic neuron activity. The method may further comprise evaluating the candidate agent in a control population of neurons (e.g., whole brain, a population of neurons other than that being screened).

Methods of evaluating polynucleotide and/or polypeptide expression are well known in the art and/or described herein. By way of example, polynucleotide microarrays maybe utilized. The candidate agent may alter expression of the drug target at any level including, but not limited to, modulating transcription of a polynucleotide drug target (e.g., a candidate agent that binds to the upstream controlling region) and/or modulating translation of the polynucleotide (e.g., an anti- sense polynucleotide, or a candidate agent which selectively degrades or stabilizes the mRNA, or by binding to drug target).

In another embodiment, a method of assessing the ability of a candidate agent to modulate dopaminergic and/or noradrenergic neuron activity and/or function comprises: (a) contacting a population of dopaminergic and/or noradrenergic neurons expressing one or more polynucleotide and/or polypeptide drug targets with a candidate agent and (b) evaluating the activity and/or function of the population of dopaminergic and/or noradrenergic neurons, wherein an alteration in the dopaminergic and/or noradrenergic neuron activity and/or function indicates the possible therapeutic potential of the candidate agent for treating one or more diseases or disorders associated with dopaminergic and/or noradrenergic neuron activity or one or more symptoms associated with dopaminergic and/or noradrenergic neuron activity. The method may further comprise evaluating the candidate agent in a control population of neurons (e.g., whole brain, a population of neurons other than that being screened).

Examples of parameters to measure to evaluate an alteration in dopaminergic and/or adrenergic function and/or activity when contacted with a candidate agent include, but are not limited to, gross phenotypic changes in the dopaminergic and/or adrenergic neurons, alteration in dopamine uptake in dopaminergic neurons, neuronal excitability (Abeliovich et al. (2000) Neuron 25(1):239-52), neuronal survival, behaviorial changes or other deficits.

The candidate agent may be evaluated on dopaminergic and/or noradrenergic neurons in vitro or in vivo. In vitro systems include, but are not limited to cell cultures, such as primary cultures of dopaminergic and/or noradrenergic neurons. By way of example, primary cultures of dopaminergic and/or noradrenergic neurons may be used (e.g., Hynes et al. (1994) J. Neuroscience Res. 37:144-154; Poulsen et al. (1994) Neuron 13:1245-1252; Masuko, S. et al (1986) J. Neurosci. 6(11):3229-41).

Alternatively, in vivo systems may be used in the screen. Any animal may be used for the screening method. Examples include, but are not limited to, drosophilia, zebrafish, rodents, such as mice or rats, or primates. The animal used in the screening method may naturally express one or more of the polynucleotide and/or polypeptide drug targets or transgenic animals expressing one or more of the polynucleotide and/or polypeptide drug targets may be generated by methods known in the art. Animal disease model systems may also be used. By way of example, mouse and rat models for Parkinson's disease include injection of 6-hydroxydopamine into the substantia nigra (rats, mice, cats or primates); intravenous infusion of Rotenone (rats), acute and chronic MTPT administration (mice, rats, primates) and mice or drosophila overexpressing alpha synuclein (Beal (2001) Nat Rev Neurosci. 2(5):325-34.).

Any population of dopaminergic and/or neuroadrenergic neurons may be used in the screen. By way of example and not limitation, nigrostiatal dopaminergic neurons (e.g., substantia nigra), mesolimbic dopaminergic neurons (e.g., ventral tegmental area) and /or mesocotical dopaminergic neurons (e.g., ventral tegmental area) or noradrenergic neurons of the locus coeruleus may be screened by the methods described herein.

In yet another embodiment, a method of assessing the ability of a candidate agent to bind to one or more of the polynucleotide and/or polypeptide drug target identified by the methods described herein is provided. The method comprises, (a) contacting one or more of the polynucleotide and/or polypeptide drug targets for dopaminergic and/or noradrenergic neurons with a candidate agent and (b) evaluating the binding of the candidate agent to the polynucleotide and/or polypeptide drug target, wherein the ability of the candidate agent to bind to the drug target is indicative of the possible therapeutic potential of the candidate agent for treating one or more diseases or disorders associated with dopaminergic and/or noradrenergic neuron activity or one or more symptoms associated with dopaminergic and/or noradrenergic neuron activity.

The drug targets to be used in assessing the ability of a candidate agent to bind to a drug target may be any one or more of the polynucleotide drug targets and/or one or more of the polypeptide drug target identified by the methods described herein. The choice of drug target will generally be directed by the population of neurons implicated in the dopaminergic and/or noradrenergic disease or disorder of interest. By way of example, for substania nigra dopaminergic cells one or more of the polynucleotides drug targets referenced in FIG. 8, FIG. 9, FIG. 10, FIG. 11, FIG. 13, FIG. 14, FIG. 15, Table 1, Table 2 and Table 4 and/or their corresponding polypeptide drug targets may be utilized, for zona incerta A13 neurons one or more of the polynucleotide drug targets referenced in FIG. 5, FIG. 13, FIG. 14 and Table 1 and/or their corresponding polypeptide drug target may be utilized, for ventral tegmental area neurons one or more of the polynucleotide drug targets referenced in FIG. 7, FIG. 9, FIG. 10, FIG. 13, FIG. 14, FIG. 15, Table 1, Table 2 and Table 4 and/or their corresponding polypeptide drug target may be utilized, and for neurons in the locus coeruleus one or more of the polynucleotide drug targets referenced in FIG. 6, FIG. 12, FIG. 13, FIG. 14, Table 1 and Table 3 and/or their corresponding polypeptide drug targets may be utilized or combinations thereof.

By way of example, and not limitation, the ability of a candidate agent to bind to a drug target may be assessed by recombinantly expressing a polynucleotide encoding a drug target in a prokaryotic or eukaryotic expression system as a native or as a fusion protein in which a drug target polypeptide (or fragment thereof) is conjugated with a well-characterized epitope or protein as are well known in the art. Recombinant drug target polypeptide is then purified by, for instance, by immunoprecipitation using an antibody specific for the drug target or anti-epitope antibodies or by binding to immobilized ligand of the conjugate. An affinity column made of drug target polypeptide or drug target polypeptide fusion protein is then used to screen a mixture of candidate agents which have been appropriately labeled. Suitable labels include, but are not limited to fluorochromes, radioisotopes, enzymes and chemiluminescent compounds. The unbound and bound compounds can be separated by washes using various conditions (e.g. high salt, detergent) that are routinely employed by those skilled in the art. Non-specific binding to the affinity column can be minimized by pre-clearing the compound mixture using an affinity column containing merely the conjugate or the epitope. A similar method can be used for screening for agents that competes for binding to the drug target polypeptide. In addition to affinity chromatography, there are other techniques such as measuring the change of melting temperature or the fluorescence anisotropy of a protein which will change upon binding another molecule. For example, a BIAcore assay using a sensor chip (supplied by Pharmacia Biosensor, Stitt et al. (1995) Cell 80: 661-670) that is covalently coupled to native drug target or drug target fusion proteins, may be performed to determine the drug target polypeptide binding activity of different agents. Polypeptide microarrays comprising one or more of the polypeptide drug targets or fragments thereof attached to a support may also be used to screen for candidate agents capable of binding to the d to one or more polypeptide drug targets.

For an assay that determines whether a candidate agent inhibits transcription of a polynucleotide drug target, an in vitro transcription or transcription/translation system may be used. These systems are available commercially, and generally contain a coding sequence as a positive, preferably internal, control. A drug target polynucleotide is introduced and transcription is allowed to occur. Comparison of transcription products between an in vitro expression system that does not contain any agent (negative control) with an in vitro expression system that does contain a candidate agent indicates whether a candidate agent is affecting transcription of the drug target polynucleotide. Comparison of transcription products between the control and the drug target polynucleotide indicates whether the agent, if acting on this level, is selectively affecting transcription of the drug target polynucleotide (as opposed to affecting transcription in a general, non-selective or specific fashion).

For an assay that determines whether a candidate agent inhibits translation of a polynucleotide drug target, an in vitro transcription/translation assay as described above may be used, except the translation products are compared. Comparison of translation products between an in vitro expression system that does not contain any candidate agent (negative control) with an in vitro expression system that does contain a candidate agent indicates whether the agent is affecting polynucleotide drug target transcription. Comparison of translation products between control and the drug target polynucleotide indicates whether the candidate agent, if acting on this level, is selectively affecting translation of the drug target polynucleotide(as opposed to affecting translation in a general, non-selective or specific fashion).

In another embodiment, competition assays are utilized. By way of example, an in vitro screening assay detects agents that compete with another substance (most likely a polypeptide) that binds a drug target polypeptide. Competitive binding assays are known in the art and need not be described in detail herein. Briefly, such an assay entails measuring the amount of a drug target polypeptide complex formed in the presence of increasing amounts of the putative competitor. For these assays, one of the reactants is labeled using, for example, ³²P.

By way of example, the ability of a candidate agent to modulate function or activity may be evaluated by, but are not limited to, gross phenotypic changes in the dopaminergic and/or adrenergic neurons, alteration in dopamine uptake in dopaminergic neurons, neuronal excitability (Abeliovich et al. (2000) Neuron 25(1):239-52), neuronal survival, behaviorial changes or other deficits.

It is also understood that the screening methods of this invention include structural, or rational, drug design, in which the amino acid sequence, three-dimensional atomic structure or other property (or properties) of a drug target 32 polynucleotide or drug target polypeptide provides a basis for designing a candidate agent which is expected to bind to a drug target polynucleotide or polypeptide. Generally, the design and/or choice of agents in this context is governed by several parameters, such as the perceived function of the polynucleotide or polypeptide target, its three-dimensional structure (if known or surmised), and other aspects of rational drug design. Techniques of combinatorial chemistry can also be used to generate numerous permutations of candidate agents. For purposes of this invention, an agent designed and/or obtained by rational drug designed may also be tested in any of the methods described herein.

By way of example, the ability of a candidate agent to modulate function or activity may be evaluated by, but are not limited to, gross phenotypic changes in the dopaminergic and/or adrenergic neurons, alteration in dopamine uptake in dopaminergic neurons, neuronal excitability (Abeliovich et al. (2000) Neuron 25(1):239-52), neuronal survival, behaviorial changes or other deficits. Examples of parameters to measure to evaluate an alteration in dopaminergic and/or adrenergic function and/or activity when contacted with a candidate agent include, but are not limited to, gross phenotypic changes in the dopaminergic and/or adrenergic neurons, alteration in dopamine uptake in dopaminergic neurons, neuronal excitability (Abeliovich et al. (2000) Neuron 25(1):239-52), neuronal survival, behaviorial changes or other deficits.

The screening methods generally require comparison to a control sample to which no agent is added. The screening methods described above generally represent primary screens, designed to detect any agent that may the desired activity. The skilled artisan will recognize that secondary tests may be necessary in order to evaluate an agent further. For example, a cytotoxicity assay would be performed as a further corroboration that an agent which tested positive in a primary screen would be suitable for use in living organisms. Any assay for cytotoxicity would be suitable for this purpose, including, for example the MTT assay (Promega).

The drug targets identified herein may be used to generate transgenic animals or knockout animals by methods known in the art. By way of example, a knockout line(s) based on one or more drug targets identified herein will allow for assessment of phenotypic changes in the appearance number of dopaminergic and/or noradrenergic neurons (Cacalano et al. (1998) Neuron (21)1:53-62), behavior (Abeliovich et al. (2000) Neuron 25(1):239-252) of heterozygotes and homozygotes following birth and later stages of development. Standard histological methods can be used to compare homozygous and wild type animals at several stages throughout embryonic development (Moore et al. (1996) Nature 382(6586):76-79). Alternatively, the consequence of over expression or down regulation of the drug targets on dopamine release, dopamine reuptake and neuronal excitability can be evaluated in transgenic animals or in vitro cultures.

### Microarrays for Screening

The polynucleotide drug targets identified by the methods described herein are useful in the screening assays described herein. The screening method can be performed as described herein to detect polynucleotide sequences from the system in which the candidate agent was tested, which are complementary to the polynucleotide drug targets. By way of example, for the screening method the polynucleotide sequences to be evaluated (e.g., polynucleotide drug targets) may comprise an array of one or more polynucleotide drug targets immobilized on a support (e.g., dot blots on a nylon hybridization membrane Sambrook et al., or Ausubel et al) that is contacted with polynucleotides isolated from the system in which the candidate was evaluated. The one or more polynucleotide drug targets immobilized on the support may comprise all or part (e.g., a functional domain) of a coding region and/or non-coding sequences. One or more of the polynucleotide drug targets referenced in FIGS. 5-15 and Tables 1-4 and/or their complement represent drug targets which may be used for the microarray. By way of example, at least 2, 3, 5, 10, 20, 40, 50, 60, 70, 80, 90, 100, 200, 300 or 400 of the polynucleotide drug targets referenced in FIGS. 5-15 and Tables 1-4 and/or their complement may comprise the microarray. In some embodiments, 20, 30, 40 or 50 of the polynucleotide drug targets referenced in FIGS. 5-15 and Tables 1-4 and/or their complement may comprise the microarray. In some embodiments, 60, 70, 80, 90, 100, 200, 300 or 400 of the polynucleotide drug targets referenced in FIGS. 5-15 and Tables 1-4 and/or their complement may comprise the microarray.

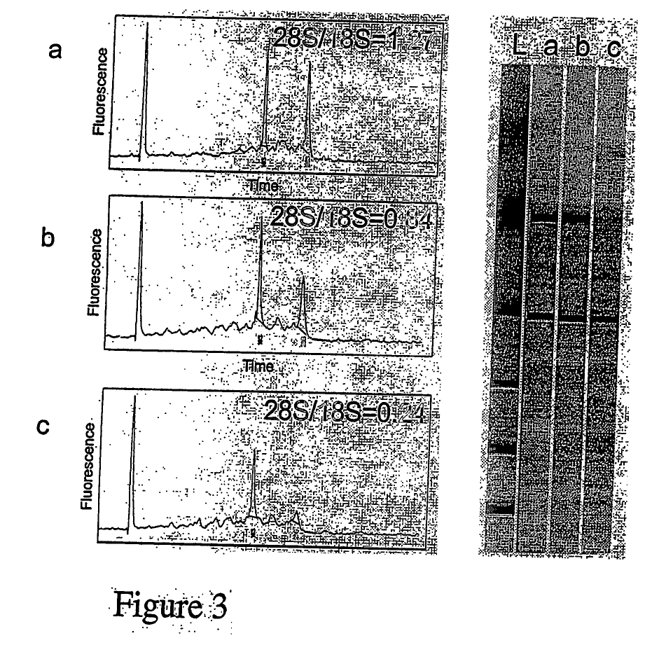

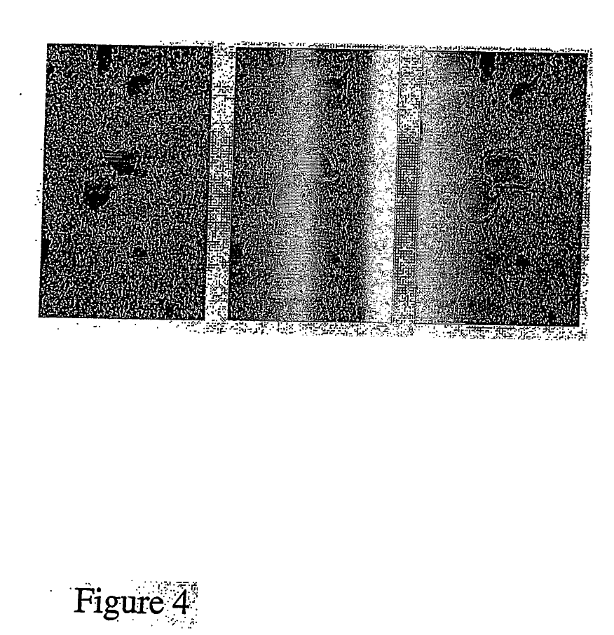

The choice of the one or more polynucleotide drug targets comprising the microarray will generally be directed by the population of neurons implicated in the dopaminergic and/or noradrenergic disease or disorder of interest. By way of example, the microarray may comprise one or more of the polynucleotide drug targets referenced in FIG. 3, FIG. 9, FIG. 10, FIG. 11, FIG. 13, FIG. 14, FIG. 15, Table 1, Table 2 and Table 4 for screening for an agent that modulates drug target expression in substania nigra, one or more of the polynucleotide drug targets referenced in FIG. 5, FIG. 13, FIG. 14 and Table 1 for screening for an agent that modulates drug target expression in zona incerta A13 neurons, one or more of the polynucleotide drug targets referenced in FIG. 7, FIG. 9, FIG. 10, FIG. 13, FIG. 14, FIG. 15, Table 1, Table 2 and Table 4 for screening for an agent that modulates drug target expression in ventral tegmental area neurons, one or more of the polynucleotide drug targets referenced in FIG. 6, FIG. 12, FIG. 13, FIG. 14, Table 1 and Table 3 for screening for an agent that modulates drug target expression in the locus coeruleus or combinations thereof.

In one embodiment, the microarray may comprise 2, 3, 5, 10, 20, 40, 50, 60, 70, 80, 90, 100, 200, 300 or 400 of the polynucleotide drug targets or their complements for substania nigra neurons, zona incerta A13 neurons, ventral tegmental area neurons or locus coeruleus neurons or combinations thereof. In some embodiments, 20, 30, 40 or 50 of the polynucleotide drug targets or their complements for substania nigra neurons, zona incerta A13 neurons, ventral tegmental area neurons or locus coeruleus neurons or combinations thereof may comprise the microarray. In some embodiments, 60, 70, 80, 90, 100, 200, 300 or 400 of the polynucleotide drug targets or their complements for substania nigra neurons, zona incerta A13 neurons, ventral tegmental area neurons or locus coeruleus neurons or combinations thereof may comprise the microarray.

Microarrays may be a solid phase on the surface of which are immobilized a population of the polynucleotides of the invention. Microarrays can be generated in a number of ways. The one or more polynucleotide drug targets can be immobilized on solid support or surface, which may be made from, for example, glass, plastic (e.g., polypropylene, nylon), polyacrylamide, nitrocellulose, or other materials. Methods for attaching the nucleic acids to the surface of the solid phase include, but are not limited to, printing on glass plates (Schena et al. (1995) Science 270:467-470; DeRisi et al. (1996) Nature Genetics 14:457-460; Shalon et al. (1996) Genome Res. 6:639-645; and Schena et al. (1995) Proc. Natl. Acad. Sci. (U.S.A.) 93:10539-11286); or ink jet printer.

The microarrays can also be high-density oligonucleotide arrays. Techniques are known for producing arrays containing thousands of oligonucleotides complementary to defined sequences (see, Fodor et al. (1991) Science 251:767-773; Pease et al. (1994) Proc. Natl. Acad. Sci. U.S.A. 91:5022-5026; Lockhart et al. (1996) Nature Biotechnology 14:1675; U.S. Pat. Nos. 5,578,832; 5,556,752; and 5,510,270; Blanchard et al. Biosensors & Bioelectronics 11:687-690). Other methods for making microarrays may also be utilized (Maskos and Southern, (1992) Nuc. Acids. Res. 20:1679-1684; U.S. Pat. No. 6136592; WO 200054883; WO 200055363; WO 200053812; WO 200014273). The microarrays may be used as is or incorporated into a biochip, multiwell or other device. In general, the oligonucleotide probes range from about 6, 8, 10, 12, 15, 20, 30 to about 100 bases long, with from about 10 to about 80 bases being preferred, and from about 30 to about 50 bases being particularly preferred.

Preferably the microarrays of the present invention comprise, polynucleotides or fragments thereof from, for example, FIGS. 5-15 and Tables 1-4. One of skill in the art will understand that the hybridization and wash conditions are chosen so that the nucleic acid sequences to be analyzed by the invention (e.g., the nucleic acids isolated from the test system) “specifically bind” or “specifically hybridize” to the nucleic acid sequences the array. Optimal hybridization conditions will depend on the length (e.g., oligomer versus polynucleotide greater than 200 bases) and type (e.g., RNA, or DNA) of probe and target nucleic acids. General parameters for specific (i.e., stringent) hybridization conditions for nucleic acids are described in Sambrook et al., (supra), and in Ausubel et al., 2001, “Current Protocols in Molecular Biology,” Greene Publishing and Wiley-Interscience, New York).

Microarrays comprising one or more of the polypeptide drug targets or fragments thereof identified by the methods described herein are also useful in, for example, a screening assay to detect a candidate agent that binds to a polypeptide drug target. One or more of the polypeptide drug targets may be immobilized on a support that is contacted with a candidate agent. Methods for generating polypeptide microarrays and methods for evaluating binding of candidate agents to the polypeptides comprising the microarray are know in the art (see, e.g., U.S. Patent Application Nos.: 2003/0049626, 2002/0106702, 2003/0013130, 2002/0110933; Koch et al (eds) Peptide Arrays on Membrane Supports: Synthesis and Applications (June 2002) Springer-Verlag).

The one or more polypeptide drug targets immobilized on the support may comprise an entire protein or portion thereof (e.g., functional or active domain). One or more of the polypeptide drug targets encoded by the polynucleotide drug targets referenced in FIGS. 5-15 and Tables 1-4 thereof may be used for the microarray. By way of example, at least 2, 3, 5, 10, 20, 40, 50, 60, 70, 80, 90, 100, 200, 300 or 400 of the polypeptide drug targets encoded by the polynucleotide drug targets referenced in FIGS. 5-15 and Tables 1-4 may comprise the microarray. In some embodiments, 20, 30, 40 or 50 of the polypeptide drug targets encoded by the polynucleotide drug targets referenced in FIGS. 5-15 and Tables 1-4 may comprise the microarray. In some embodiments, 60, 70, 80, 90, 100, 200, 300 or 400 of the polypeptide drug targets encoded by polynucleotide drug targets referenced in FIGS. 5-15 and Tables 1-4 may comprise the microarray.

As for the polynucleotide microarray, the choice of the one or more, polypeptide drug targets comprising the microarray will generally be directed by the population of neurons implicated in the dopaminergic and/or noradrenergic disease or disorder of interest. By way of example, the microarray may comprise one or more of the polypeptide drug targets encoded by a polynucleotide referenced in FIG. 8, FIG. 9, FIG. 10, FIG. 11, FIG. 13, FIG. 14, FIG. 15, Table 1, Table 2 and Table 4 for screening substania nigra neurons, one or more of the polypeptide drug targets encoded by a polynucleotide referenced in FIG. 5, FIG. 13, FIG. 14 and Table 1 for screening zona incerta A13 neurons, one or more of the polypeptides drug targets encoded by a polynucleotide referenced in FIG. 7, FIG. 9, FIG. 10, FIG. 13, FIG. 14 or FIG. 15 and Tables 1, Table 2 or Table 4 for screening in ventral tegmental area neurons, one or more of the polypeptide drug targets encoded by a polynucleotide referenced in FIG. 6, FIG. 12, FIG. 13, FIG. 14, Table 1 and Table 3 for screening in locus coeruleus neurons. In one embodiment, the microarray may comprise 2, 3, 5, 10, 20, 40, 50, 60, 70, 80, 90, 100 or 200 of the polypeptide drug targets for substania nigra neurons, zona incerta A13 neurons, ventral tegmental area neurons or locus coeruleus neurons or combinations thereof. In some embodiments, 20, 30, 40 or 50 of the polypeptide drug targets for substania nigra neurons, zona incerta A13 neurons, ventral tegmental area neurons or locus coeruleus neurons or combinations thereof may comprise the microarray. In some embodiments, 60, 70, 80, 90, 100 or 200 of the polypeptide drug targets for substania nigra neurons, zona incerta Al 3 neurons, ventral tegmental area neurons or locus coeruleus neurons or combinations thereof may comprise the microarray.

### Candidate Agents

Candidate agents suitable for assaying in the methods of the subject application may be any type of molecule from, for example, chemical, nutritional or biological sources. The agent may be a naturally occurring or synthetically produced. For example, the agent may encompass numerous chemical classes, though typically they are organic molecules, preferably small organic compounds having a molecular weight of more than 50 and less than about 2,500 Daltons. Such molecules may comprise functional groups necessary for structural interaction with proteins or nucleic acids. By way of example, chemical agents may be novel, untested chemicals, agonists, antagonists, or modifications of known therapeutic agents.

The agents may also be found among biomolecules including, but not limited to, peptides, saccharides, fatty acids, antibodies, steroids, purines pryimidines, derivatives or structural analogs thereof or a molecule manufactured to mimic the effect of a biological response modifier. Examples of agents from nutritional sources include, but is not limited to, extracts from plant or animal sources or extracts thereof.

Agents may be obtained from a may be obtained from a wide variety of sources including libraries of synthetic or natural compounds. Alternatively, libraries of natural compounds in the form of bacterial, fungal, plant, and animal extracts are available or readily produced. Additionally, natural or synthetically produced libraries or compounds are readily modified through conventional chemical, physical and biochemical means, and may be used to produce combinatorial libraries. Known pharmacological agents may be subjected to random or directed chemical modifications, such as acylation, alkylation, esterification, amidification, etc. to produce structural analogs.

Exemplary types of candidate agents that may be screened in the methods include, but are not limited to, an antibody, an anti-sense molecule, a structural analog of a drug target, a dominant-negative mutation of a drug target, an immunoadhesion, and small molecules having a molecular weight of 100 to 20,000 daltons, 500 to 15,000 daltons, or 1000 to 10,000 daltons. Libraries of small molecules are commercially available.

By way of example, polynucleotides may be candidate agents. Examples of polynucleotides include but is not limited to, single-, double-stranded and triple helical molecules, a gene or gene fragment, exons, introns, mRNA, tRNA, rRNA, siRNA (small interfering RNAs), ribozymes, antisense, cDNA, recombinant polynucleotides, branched polynucleotides, plasmids, vectors, isolated DNA of any sequence, isolated RNA of any sequence, nucleic acid probes, and primers. The polynucleotide may comprise naturally occurring nucleic acid molecules, synthetic or modified nucleic acid molecules.

### Antibodies as Candidate Agents

The candidate agent may be an antibody which specifically binds one or more of the drug targets. The antibodies can be monoclonal antibodies, polyclonal antibodies, antibody fragments (e.g., Fab, Fab′, F(ab′)2, Fv, Fc, etc.), chimeric antibodies, bispecific antibodies, heteroconjugate antibodies, single chain (ScFv), mutants thereof, fusion proteins comprising an antibody portion, humanized antibodies, and any other modified configuration of the immunoglobulin molecule that comprises an antigen recognition site of the required specificity, including glycosylation variants of antibodies, amino acid sequence variants of antibodies, and covalently modified antibodies. The antibodies may be murine, rat, human, or any other origin (including chimeric or humanized antibodies). The epitope(s) can be continuous or discontinuous. In one aspect, antibodies (e.g., human, humanized, mouse, chimeric) that can be made by using immunogens that express all or part of a polynucleotide encoding a drug target. In another aspect, an immunogen comprising a cell that overexpresses a drug target. Another example of an immunogen that can be used is all or part of a polypeptide drug target. The antibodies may be made by any method known in the art and tested by known methods. In an alternative, antibodies may be made recombinantly and expressed using any method known in the art. In another alternative, antibodies may be made recombinantly by phage display technology. See, for example, U.S. Pat. Nos. 5,565,332; 5,580,717; 5,733,743; 6,265,150; and Winter et al., Annu. Rev. Immunol. 12:433-455 (1994). Alternatively, the phage display technology (McCafferty et al., Nature 348:552-553 (1990)) can be used to produce human antibodies and antibody fragments in vitro, from immunoglobulin variable (V) domain gene repertoires from unimmunized donors.

### Kits

The invention also provides kits for use in the instant methods. Kits of the invention include one or more containers comprising one or more polynucleotide and /or polypeptide drug targets provided by the method described herein, in the form of, for example, a microarray or antibodies. The kit may further comprise instructions for any of the screening method. The kit of this invention are in suitable packaging. Suitable packaging includes, but is not limited to, vials, bottles, jars, flexible packaging (e.g., sealed Mylar or plastic bags), and the like. In some embodiments, the kit comprises a container and a label or package insert on or associated with the container. The container holds a composition which is effective for use in the methods described herein. The container may further comprise an active agent. In another embodiment, the kit may comprise two or more containers each containing a composition effective for the methods described herein.

By way of example the kit may comprise one or more polynucleotide and/ or polypeptide microarrays as described above, wherein the one or more microarray comprises gene expression profiles for substania nigra neurons, zona incerta A13 neurons, ventral tegmental area neurons, and/or locus coeruleus neurons or combinations thereof. The gene expression profiles may be combined on a single microarray or two or more microarrays.

By way of example, the microarray may comprise one or more of the polynucleotide drug targets referenced in FIG. 8, FIG. 9, FIG. 10, FIG. 11, FIG. 13, FIG. 14, FIG. 15, Table 1, Table 2 and 4 for screening for an agent that modulates drug target expression in substania nigra, one or more of the polynucleotide drug targets referenced in FIG. 5, FIG. 13, FIG. 14 and Table 1 for screening for an agent that modulates drug target expression in zona incerta A13 neurons, one or more of the polynucleotide drug targets referenced in FIG. 7, FIG. 9, FIG. 10, FIG. 13, FIG. 14, FIG. 15, Table 1, Table 2 and Table 4 for screening for an agent that modulates drug target expression in ventral tegmental area neurons, one or more of the polynucleotide drug targets referenced in FIG. 6, FIG. 12, FIG. 13, FIG. 14, Table 1 and Table 3 for screening for an agent that modulates drug target expression in the locus coeruleus or combinations thereof.

By way of example, the microarray may comprise one or more of the polypeptide drug targets encoded by a polynucleotide referenced in FIG. 8, FIG. 9, FIG. 10, FIG. 11, FIG. 13, FIG. 14, FIG. 15, Table 1, Table 2 and Table 4 for screening substania nigra neurons, one or more of the polypeptide drug targets encoded by a polynucleotide referenced in FIG. 5, FIG. 13, FIG. 14 and Table 1 for screening zona incerta A13 neurons, one or more of the polypeptides drug targets encoded by a polynucleotide referenced in FIG. 7, FIG. 9, FIG. 10, FIG. 13, FIG. 14 or FIG. 15, Table 1, Table 2 and Table 4 for screening in ventral tegmental area neurons, one or more of the polypeptide drug targets encoded by a polynucleotide referenced in FIG. 6, FIG. 12, FIG. 13, FIG. 14, Table 1 and Table 3 for screening in locus coeruleus neurons.

Alternatively the kit, for example, may comprise primers for amplifying the polynucleotide drug targets and/or antibodies which bind the polypeptide drug targets.

The following examples illustrate the manner in which the invention can be practiced. It is understood, however, that the examples are for the purpose of illustration and the invention is not to be regarded as limited to any of the specific materials or conditions therein.

### EXAMPLES

### Example 1

### Drug Targets Identified In Rat Brain Tissue

Tissue Preparation and Immunohistochemistry

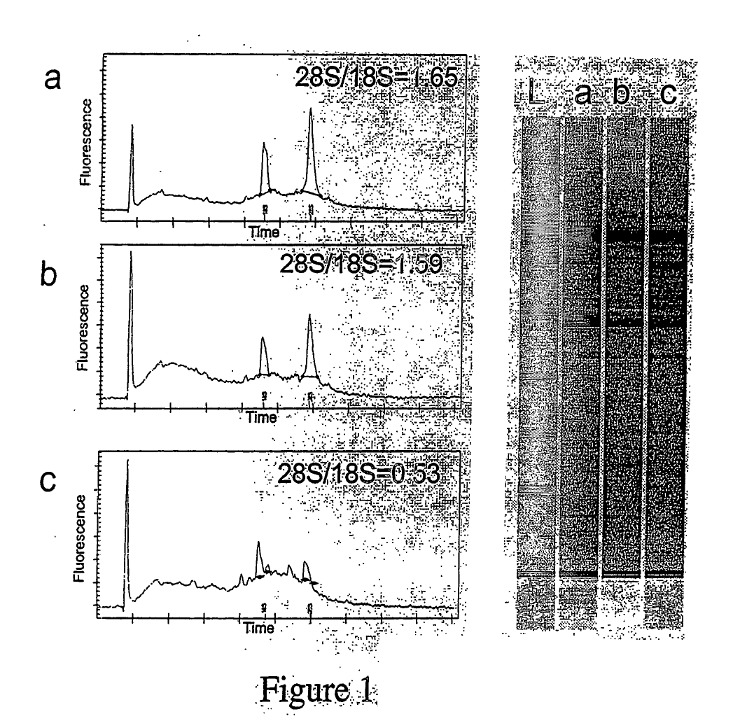

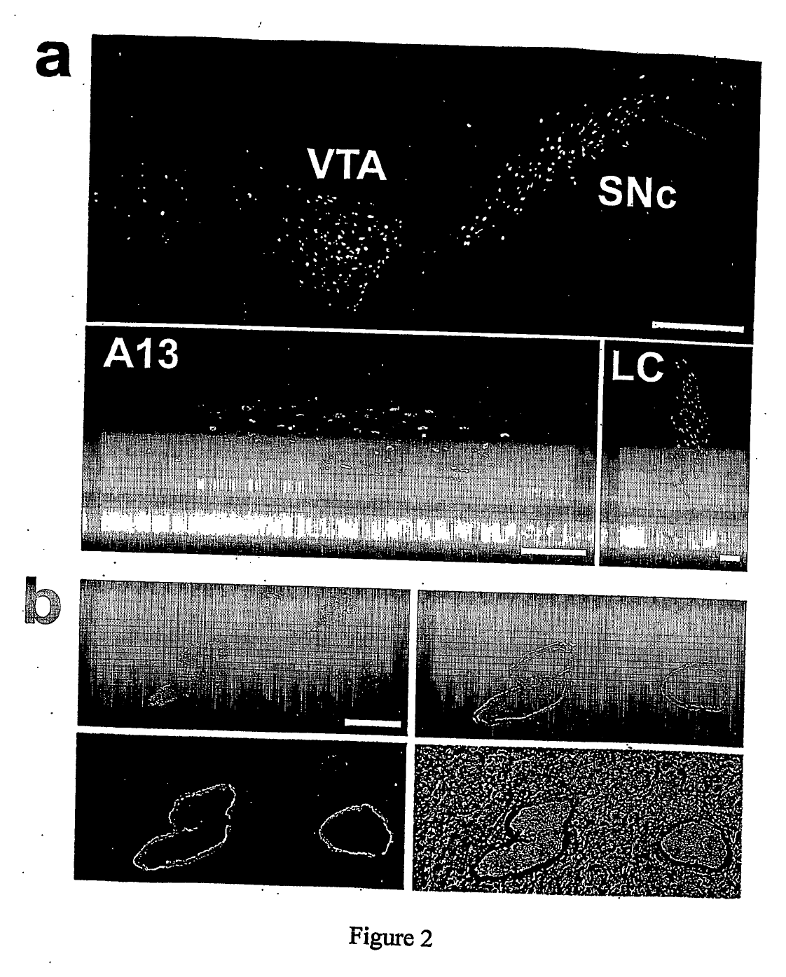

Standard histochemistry protocols result in severely degraded RNA that is not suitable for RNA amplification and microarray analysis. Incubation of tissue sections in buffered aqueous solutions results in nearly complete degradation after only several minutes. In order to allow microarray analysis of immunostained single cells a staining protocol that results in only minimal degradation of RNA was developed. The method involves a rapid 4-minute staining protocol that allows antigen detection with high sensitivity without severely compromising RNA integrity (FIGS. 1 and 2).

Rat brains are dissected and immediately and allow to freeze slowly on dry ice. Frozen brain specimen are stored at −80° C. until sectioning. 12 μm serial sections are cut in the cryostat and are mounted on pre-processed polyethylene naphthalene membrane slides (see below). The sections are fixed immediately in 100% ethanol for 30 s followed by a dip in Acetone for 2 seconds and air dried at RT. The sections are rehydrated in PBS, pH7.0 containing 1 U/ul RNAse inhibitor (Roche, Germany) for 5 seconds. The sections are stained with 100 μg/ml labeled anti tyrosine hydroxylase antibody (see below) in PBS pH7 containing 1 U/ul RNAse inhibitor (Roche, Germany) for 3 min followed by two washes in PBS, pH7.0 for 5 seconds. The sections are then dehydrated for 30 s in 75%, 95%, and 100% ethanol respectively and air-dried at room temperature. (FIG. 1).

Processing of Slides for Laser Microdissection

Membrane slides were engineered for maximal laser cutting and catapulting efficiency: A 1.35 μm polyethylene naphthalene membrane is sealed to the slide with 0.1% poly-L-lysine followed by UV irradiation for 30 minutes. To overcome the hydrophobic nature of the membrane and improve adherence of the tissue section, the membrane coated glass slides are incubated again in 0.1% poly-L-lysine for about 5 minutes, spun dry and allowed to air dry for 1 hour.

Generation of Alexa Fluor 488 Labeled Antibody

Tyrosine hydroxylase positive cells are detected with anti-tyrosine hydroxylase ascites fluid (clone TH-16, Sigma, USA). The antibody is purified with a protein A column and is covalently labeled with Alexa 488 fluorophore using the Alexa Fluor 488 monoclonal antibody labeling kit according to manufacturer's instructions. The labeled antibody is purified via gel filtration column chromatography followed by three washes with PBS in a Microcon 30 centrifugal filter device.

Laser Microdissection and RNA Isolation

A PALM Robot-Microbeam system (PALM microlaser technology, Germany) for isolation of single neurons from frozen sections of brain tissues was used. The technology allows efficient contact-free isolation of cells of any size and shape while minimizing the risk of contamination. The selected cells are circumscribed with a high energy focused nitrogen laser resulting a gap of several microns in which any biological material has been ablated. The morphology of the adjacent tissue is not compromised by that procedure. Following laser-microdissection, the laser is focused slightly below the dissected target, which is then ejected from the object slide by photonic pressure of a second laser pulse and collected in a microcap containing RNA lysis buffer. To facilitate detection of fluorescent cells, a drop of 100% ethanol is applied to the section while the cells are selected. Sections are allowed to air dry again and 200 cells are dissected and catapulted into 30 μl of lysis buffer. Total RTA is isolated using silica matrix-based RNA isolation kit (Picopure Kit, Arcturus) contaminating genomic DNA is removed during the isolation by an on-column DNAse digestion step.

RNA Amplification

RNA was amplified by two rounds of T7-based linear amplification (Van Gelder et al., 1990). In this procedure, the MRNA is converted into cDNA using an oligo-dT primer that contains a T7 RNA polymerase promoter site. The double-stranded cDNA is used as template for T7 RNA polymerase to transcribe antisense RNA which is amplified up to 1000 fold compared to the original input MRNA. The antisense RNA is used for a second round of amplification resulting in about 10⁶-fold amplification. For amplification, the Riboamp kit (Arcturus) was used according to the manufacturer's protocol with the following modifications: To minimize generation of template-independent amplification product from the T7 primer, a five fold dilution of primer A was used for first round cDNA synthesis and the reaction volume was scaled down by 50%. The yield and size distribution of the amplified aRNA product is evaluated by microfluidic gel electrophoresis with the Agilent bioanalyzer.

Preparation and Hybridization of Fluorescent Labeled cDNA

For each comparative array hybridization, labeled cDNA was synthesized by reverse transcription from amplified RNA from isolated neurons in the presence of Cy5-dUTP, and from the whole brain reference mRNA with Cy3-dUTP, using the Superscript II reverse-transcription kit (Gibco-BRL). For each reverse transcription reaction, 2 μg RNA was mixed with 3 μg random hexamers (Invitrogen) in 16 μl H2O, heated to 70° C. for 10 min and cooled on ice. To this sample, we added an 0.6 μl unlabelled nucleotide pool (20 mM each DATP, dCTP, dGTP; 4 mM dTTP and 16 mM aminoallyl-dUTP), 6 μl 5×first-strand buffer, 3 μl 0.1 M DTT and 2 μl of Superscript II reverse transcriptase (200 U/μl). The reaction was incubated five minutes at 25° C. followed by one hour at 37° C. and one hour at 42° C. The RNA was then degraded by adding 15 μl 1 N NaOH and incubating at 70° C. for 10 min and neutralized by addition of 15 μl 1 N HCl. The cDNA was purified by three rounds of centrifugation in a Centricon-30 micro-concentrator (Amicon). Each time 450 ul of H₂O was added and the reaction was concentrated to 20 μl. The purified samples were dried in a vacuum concentrator and reconstituted in 10 μl of 50 mM Na₂CO₃ (pH 9). 1 μl of monofunctional NHS-ester Cy3 or Cy5 dye (Amersham, 10 mM in DMSO) was added to each sample and coupled in the dark for 1 h. Unreactive NHS-esters were quenched by addition of 4.5 μl 4 M hydroxylamine (Sigma) for 15 min in the dark. The labeled sample targets were combined with the respective reference targets and unincorporated Cy esters were removed by a silica based spin columns using the Qia-Quick PCR purification kit (Qiagen) according to manufacturer's protocol. The labeled targets were eluted in 2×30 μl elution buffer. After addition Cot1 DNA (15 μg, Gibco-BRL), yeast t-RNA (15 μg, Sigma), ployA (15 μg, Sigma) and 420 μl H₂O, the labeled targets were concentrated to 10 μl in a Centricon-30 micro-concentrator (Amicon) and 2.5 μl deposition control targets (Operon) and 12.5 μl Deposition Hybridization buffer (Agilent) were added. The targets were denatured by heating for 2 min at 98° C., centrifuged at 13,000 g for 5 min and placed on the array under a 22×22 mm glass cover slip. Microarrays were hybridized for 48 h at 65° C. in a custom slide chamber with humidity maintained by a small reservoir of H2O. Arrays were washed by submersion and agitation for S min in 0.5×SSC, 0.01% SDS, followed by 3 washes in 0.06×SSC for 3 min 3 each. The arrays were dried by centrifugation for 2 min and scanned in a microarray scanner (Agilent). Images were analyzed with Agilent's feature extraction software. Data was filtered with respect signal significance (A two tailed t-test was used to determine significance of the signal versus background). Spot with a p-value of >0.01 were omitted. Only genes for which information was available for more than 80% of arrays were included. Data was log2 transformed and analyzed using CLUSTER and Treeview (Eisen, M. B., Spellman, P. T., Brown, P. O., Botstein, D. (1998) Proc. Natl. Acad. Sci. U.S.A. 95, 14863-14868). Statistical analysis was done using the significance analysis of microarrays algorithm SAM (Tusher, V. G., Tibshirani, R. & Chu, G. (2001) Proc. Natl. Acad. Sci. U.S.A. 98, 5116-5121).

Results:

The gene expression profiles of 3 populations of dopaminergic neurons (substantia nigra pars compacta (A9 cell group), the ventral tegmental area (A10 cell group) and the zona incerta (A13 cell group)) and one population of noradrenergic cells (locus coeruleus) from adult (7-9 month) female Sprague Dawley rats were analyzed (FIG. 2). For each population 3 independent captures of 200 cells from different animals were analyzed and compared to expression of a reference RNA generated from a pool of 3 pooled whole brains from age matched female rats. Genes that showed a statistical significant difference between replicas were identified with SAM (false discovery rate <1%) and subsequently ranked by their average fold expression versus whole brain. Genes with an average expression >8 compared to the whole brain reference were selected (FIGS. 5-8). To identify genes that confer differential vulnerabilities in Parkinson's disease, genes with significant expression changes between dopaminergic neurons isolated from the substantia nigra and ventral tegmental area (FIGS. 9 and 10) were selected.

### Example 2

### Drug Targets Identified In Human Brain Tissue

Tissue

The Stanford University Medical School Brain Bank provides the brain samples under NIH and Stanford University guidelines. These samples are frozen in liquid nitrogen immediately after dissection. To evaluate the RNA quality of a sample we extract the RNA of a single cyrosection and analyze it on the Agilent bioananlyzer (FIG. 3). In general the degree of preservation of RNA in post mortem human brain samples is often poor (as assessed by the presence of the ribosomal 18S and 28S ribosomal RNA peaks) and does not directly correlate with the post mortem delay. Therefore only a small subset of autopsy material is suited for single cell microarray analysis experiments (FIG. 3).

Dopaminergic neurons in the substantia nigra and noradrenergic neurons in the locus coeruleus were identified by their content of neuromelanin pigmentation (FIG. 4). All experimental steps were carried out as described as described in Example 1 except that no immunostaining was applied.

Results:

The expression profile of dopaminergic neurons isolated from the human substantia nigra compacta and noradrenergic neurons isolated from the locus coeruleus were analyzed. 200 neuromelanin-containing neurons were isolated by laser microdissection (FIG. 4). After two rounds of linear amplification, cRNA was used to generate labeled targets that were hybridized to a cDNA microarray containing 13,000 unique human genes. As a reference, whole brain RNA (Clontec) that had been amplified likewise was used. Genes with an average fold expression >8 compared to the whole brain reference in the substantia nigra or the locus coeruleus are shown in FIGS. 11 and 12.

Validity of the Method

Many previously known marker genes were detected as highly enriched in their respective cell population in rat as well as human profiling experiments, providing a strong validation of the protocol. Dopaminergic neurons use the neurotransmitter dopamine, which is synthesized from the amino acid tyrosine by two enzymes, tyrosine hydroxylase and DOPA decarboxylase. These enzymes are specifically expressed in the catecholaminergic neurons that constitute only a small subset of neurons within the brain. These genes show a dramatic enrichment in all cell populations profiled. In addition, the genes for dopamine transporter and the presynaptic dopamine receptor D2 showed high expression over whole brain in human substantia nigra and locus coeruleus cells (probes corresponding to these genes are not present on the rat cDNA arrays).

Noradrenergic neurons signal via the neurotransmitter norepinephrine. In addition to tyrosine hydroxylase and DOPA decarboxlase, these neurons express a third enzyme, dopamine Beta -hydroxylase, that converts dopamine to norepinephrine. This enzyme is exclusively expressed in adrenergic neurons. It was found that dopamine 3-beta hydroxylase showed the highest expression among genes in purified noradrenergic neurons in humans and rats compared to the whole brain (FIG. 12). In addition, tyrosine hydroxylase and DOPA decarboxlase are highly enriched in these cells. Other previously known marker genes for dopaminergic and/or noradrenergic neurons identified in our experiments include e.g. aldehyde dehydrogenase, glutathione peroxidase, gamma-synuclein, and Ret ligand 1 (GFRalpha1).

### Example 3

### Genes That Define the Four Major Classes of Dopaminergic (DA) and Noradrenergic (NA) Neurons

Material and Method

Tissue Preparation and Immunohistochemistry

Brains of adult (7-9 month) female Sprague Dawley rats were dissected and immediately frozen on dry ice. 12 lm cryosections were mounted on polyethylene naphthalene membrane slides pretreated with 0.1% poly-L-lysine for 5 min followed by 30 min of UV irradiation. The sections were fixed immediately in 100% ethanol for 30 s followed by 3 s in acetone and air dried. After rehydration in PBS, pH7.0 for 5 s, the sections were stained for 2 min in PBS, pH7.0; containing 100 μg/ml anti tyrosine hydroxylase antibody (clone TH-16, Sigma) that had been labeled with the Alexa Fluor 488 monoclonal antibody labeling kit (Molecular Probes) according to manufacturer's instructions. Rehydration and staining were performed in the presence of 1 U/ul RNAse inhibitor (Roche, Germany). The sections were washed twice in PBS, for 5 s, dehydrated for 30 s in 75%, 95%, and 100% ethanol respectively and air-dried at room temperature.

Laser Microdissection, RNA Isolation and Amplification

Single neurons were isolated from immunostained cryosections using a PALM Robot-Microbeam system (PALM microlaser technology, Germany). To facilitate detection of fluorescent neurons, a drop of 100% ethanol was applied to the section during cell selection. The sections were allowed to air dry and neurons were dissected and catapulted into 30 μl lysis buffer. Total RNA from 200 pooled neurons was isolated using the Picopure kit (Arcturus) and contaminating genomic DNA was removed during the isolation by an on-column DNAse digestion step. The common reference RNA was generated from 3 pooled whole brains of age matched female rats. RNA was isolated using RNA-Bee (Tel-Test) followed by DNAse digestion with the DNA-free kit (Ambion). The RNA from dissected neurons and the common reference were amplified by two rounds of T7-based linear amplification (Van Gelder et al. (1990) Proc. Natl. Acad. Sci. USA 87(5):1663-7) using the Riboamp kit (Arcturus) with the following modifications: To minimize generation of template-independent amplification product from the 17 primer, a 1:5 dilution of primer A was used for first round cDNA synthesis and the reaction volume was scaled down by 50%. The yield and size distribution of the amplified RNA product was evaluated by microfluidic gel electrophoresis with the bioanalyzer (Agilent).

RNA Labeling, Microarray Hybridization and Data Analysis

Detailed protocols for probe synthesis and DNA microarray hybridization are available at http://cmgm.stanford.edu/pbrown/protocols/index.html. In short, 2 μg of amplified RNA was random primed to generate single-stranded aminoallyl-dUTP cDNA targets, which were subsequently coupled with either Cy3 (whole brain reference) or Cy5 (experimental sample). Experimental and reference samples were combined and hybridized for 48 h at 65° C. in deposition hybridization buffer (Agilent) containing 15 μg of each Cot1 DNA, (Invitrogen), yeast t-RNA and polyA (Sigma) and 2.5 μl deposition control targets (Operon) to 14,815-element rat cDNA microarrays (Agilent, G2565A). Microarrays were washed for 5 min in 0.5×SSC, 0.01% SDS, followed by 3 washes in 0.06×SSC for 3 min and scanned on an Agilent G2565AA microarray scanner. Images were analyzed using Agilent feature extraction software (version A.6.1.1). Processing included local background subtraction and a rank consistency-based probe selection for LOWESS normalization. The data was filtered with respect to signal significance. A two tailed t-test was used to determine significance of the signal versus background and spots with a p-value >0.01 in the red or green channel were omitted. Data was log₂ transformed and analysed using Cluster and Treeview (Eisen, M. B. et al (1998) Proc. Natl. Acad. Sci. U.S.A. 95, 14863-14868). Statistical analysis was done using various functions of the significance analysis of microarrays algorithm SAM (Tusher, V. G., et al. (2001) Proc. Natl. Acad. Sci. U.S.A. 98:5116-5121), with a false discovery rate set to <1%. Only genes for which information was available for more than 80% of arrays were included in the analysis. Four independent experiments were conducted for every cell type and in each experiment cells were isolated from different animals and RNA extraction, amplification, labeling and hybridization were carried out separately. The mean correlation coefficient of the expression ratios (log2 isolated neurons/whole brain) between replicates was 0.86 and values ranged from 0.81 to 0.93.

Probe templates were amplified from rat brain RNA by nested RT-PCR and T3 promoter sequences were incorporated. The sequence confirmed PCR products were used as templates for synthesis of digoxigenin labeled RNA probes. 20 μm cryosections of rat brain tissue were dried for 30 min at and fixed in 4% paraformaldehyde for 15 min. The sections were bleached in 6% H₂O₂ for 10 min, digested with 1 μg/ml proteinase K in PBS for 5 min and refixed in 4% paraformaldehyde followed by a 10 min acetylation step in 0.25% acetic anhydride/100 mM Tris pH7.5 and two washes in 2×SSC pH5. The sections were prehybridized in hybridization buffer (5×SSC pH5, 1% blocking reagent (Roche), 50% formamide, 5 mM EDTA, 0.1% Tween 20, 10% dextrane sulfate, 100 μg/ml salmon sperm DNA, 100 μg/ml tRNA, 100 μg/ml heparine) for 1 h at 65° C. and hybridized o/n at 65° C. in 100 μl hybridization buffer containing 1 μg/ml digoxigenin-labeled probe. The slides were washed at 60° C. 2×10 min in 5×SSC, 50% formamide, 2×15 min in 1×SSC and 30 min 0.2×SSC. DIG epitopes were detected with alkaline phosphatase-coupled anti-digoxigenin Fab fragments (Roche) and developed with BM purple (Roche).

Results

Validation of the Experimental Approach

The expression patterns of known key enzymes involved in dopamine and noradrenalin biosynthesis and vesicular transport were used to validate the approach. TH, the rate-limiting enzyme for the synthesis of both dopamine and noradrenalin, was the most highly enriched transcript in all three dopaminergic neuron groups examined (>150 fold compared to the whole brain reference) and the second highest in the noradrenergic neurons. In contrast, dopamine-beta hydroxylase (DBH), which catalyzes the conversion of dopamine to noradrenalin, was exclusively enriched in LC neurons (>500 fold over reference). Other catecholamine synthesis enzymes like the aromatic amino acid decarboxylase (AADC), GTP cyclohydrolase I (GTPCH I) and pterin-4-alpha carbinolamine dehydratase (PCD) and the vesicular monoamine transporter 2 (VMAT-2), which mediate the transport of monoamine neurotransmitters into synaptic vesicles, were also expressed at high levels in all catecholaminergic neuron populations. As expected, the ubiquitously expressed dihydropteridine reductase (DHPR) did not show significant enrichment in either cell population.

Lineage Relationships Between Catecholaminergic Neuronal Subclasses

Lineage relationships between the different classes of catecholaminergic neurons (CA) were determined based on the overlapping patterns of gene expression. Unsupervised hierarchical clustering (Eisen, M. B. et al (1998) Proc. Natl. Acad. Sci. U.S.A. 95, 14863-14868) was used to group the four catecholaminergic neuronal classes based on all the genes represented on the array. Independent gene expression profiles from a given cell group always clustered together, indicating the existence of specific transcriptomes in each subgroup of catecholaminergic neurons. The SN and VTA dopaminergic neurons displayed highly similar signatures of gene expression, suggesting that these anatomically adjacent cell groups are closely related at the molecular level and possibly by lineage. In contrast, the profile of incerto-hypothalamic dopaminergic neurons was only distantly related to those of the SN and VTA neurons, despite the fact that all three groups of neurons use the same transmitter. In fact, in a phylogenetic tree, hypothalamic A13 dopaminergic neurons are not significantly closer to midbrain dopaminergic neurons (DA) than the noradrenergic neurons are.

Transcripts with at least 4-fold higher expression in any one of the four cell groups compared to whole brain were further studied. Noradrenergic neurons in the LC express the highest number of specifically enriched transcripts (412) followed by SN (279) and VTA (264). Hypothalamic dopaminergic neurons expressed only 170 enriched transcripts. Of the 700 enriched genes only 44 were shared by all four catecholaminergic groups examined. Neurons of the SN and VTA shared the highest number of expressed genes. Of the 372 genes that were expressed at higher levels in either SN or VTA, 46% (171/372) were enriched in both groups of neurons. In contrast, SN and A13 neurons shared 18% (68/381), SN and LC 22% (126/565) and A13 and LC 17% (85/497) of their enriched transcripts.

In an alternative approach to assess the molecular phylogeny, the percentage of transcripts with differences between any two given cell groups were determined by significance analysis. SN and VTA differed in only 122 (<1%) of their expressed genes. In contrast there were 766 (>5%) differentially expressed transcripts between SN and A13 and 1079 (>7%) between SN and LC neurons. The highest number of genes with differential expression was observed between LC and A13 neurons (1453; >10%). Taken together, these findings demonstrate that each group of catecholaminergic neurons displays a unique set of expressed genes and support the hypothesis that SN and VTA neurons are closely related by lineage and/or function.

Transcripts Enriched in all Catecholaminergic Neurons

Transcripts that are expressed at least 4 fold higher in all catecholaminergic neurons than in whole brain were examined (FIG. 13). In addition to the expected genes involved in neurotransmitter synthesis and transport such as TH, AADC, GTPCH I, PCD and VMAT-2, the most prominent functional class were genes that counteract stress-induced cell damage. One representative in this group was the transcript coding for glutathione peroxidase, which detoxifies hydrogen peroxide using reduced glutathione. Another gene involved in preventing stress-induced damage was the 8-oxo-dGTPase MTH1, which encodes the key enzyme that counteracts oxidative stress-induced DNA damage by hydrolyzing 8-OxoGTP. Other transcripts with an enriched expression include the caspase recruitment domain-containing molecule ARC, which is a potent repressor of apoptosis and protects cells from hypoxia and oxidative stress (Neuss M. et al. (2001) J. Biol. Chem. 276:33915-22), and the oxygen-regulated protein ORP150, which is induced by hypoxia and excitatory stress and can suppress neuronal death induced by glutamate or ischemia (Tamatani M. et al. (2001) Nat. Med. 7(3):317-23).

A subset of the transcripts that are expressed more than 4 fold higher (FIG. 13 provides additional examples) in all catecholaminergic neurons than in the whole brain are provided in Table 1.

TABLE 1
Examples of targets expressed >4 fold higher in all catecholaminergic neurons

| | GB | Human | | | Ratio | Ratio | Ratio |
|---|---|---|---|---|---|---|---|
| Gene name | accession | Orthologs | Unigene | RatioSN | VTA | A13 | LC |
| argininosuccinate synthetase | M31690 | NM_054012 | Mm.3217 | 6.0 | 8.6 | 11.1 | 11.6 |
| decay accelerating factor (DAF) | AB032395 | M30142 | Rn.18841 | 12.7 | 14.0 | 9.2 | 8.6 |
| MHC class I heavy chain | X90374 | U64801 | Rn.39743 | 13.7 | 11.8 | 9.0 | 9.0 |
| cell growth regulator 11 | U66470 | NM_006569 | Rn.31842 | 10.0 | 7.5 | 5.2 | 11.0 |
| calcyon | AAF34714 | NM_015722 | Rn.27756 | 6.4 | 8.4 | 7.4 | 8.4 |
| CLIC3 | AAD16450 | NM_053603 | Rn.1838 | 5.3 | 7.2 | 5.9 | 5.2 |
| arginine methyltransferase (PRMT2) | AF169620 | NM_133182 | Mm.32020 | 6.8 | 7.8 | 8.2 | 9.9 |
| HYPOTHETICAL 38.5 kDA PROTEIN | AK078264 | BC047054 | Mm.72979 | 24.5 | 29.3 | 6.5 | 11.3 |

The potential stress promoting enzyme argininosuccinate synthetase was highly expressed in all four catecholaminergic neuronal groups. This arginine regenerating enzyme is essential for sustained production of nitric oxide. An excess of nitric oxide has been shown to be neurotoxic while inhibition of NO-synthesis has a neuro-protective effect in the MPTP model of PD (Hantraye P. et al. (1996) Nat. Med. 2(9):1017-21). Two inflammation related genes, decay accelerating factor (DAF), which can protect cells against complement-mediated damage, and MHC class I heavy chain (Linda et al. (1999) J Neuroimmunol. 101(1):76-86), showed a high uniform expression in all cell groups. These molecules could play a role in the neuroinflammatory processes believed to contribute to the degeneration of catecholaminergic neurons in PD.

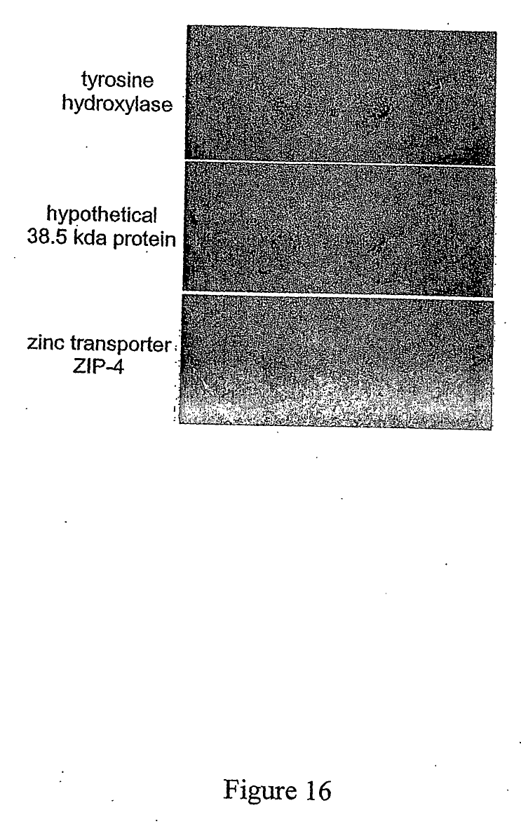

Transcripts for Neural Cell Adhesion Molecule (NCAM) along with polysialyltransferase 1, which catalyzes the addition of polysialac acid chains to NCAM and modulates its adhesive properties, were also enriched in all catecholaminergic neurons. Expression of PSA-NCAM, which is involved in the regulation of myelination as well as cell migration, axonal guidance and plasticity, is progressively lost by most tissues during development but appears to be retained in all adult catecholaminergic neuronal classes. Two genes that are associated with modulation of dopamine receptor activity, Calcyon and CLIC3 were also detected in all catecholaminergic neuronal classes. Calcyon is a cross-talk accessory protein which enables the typically Gs-linked D1/D5 dopamine receptor to stimulate intracellular calcium release (Lezcano et al. (2000) Science 287(5458):1660-4). CLIC3 belongs to the family of intracellular choride channels that are involved in a variety of cellular events including secretion, cell division and apoptosis. Another member of this family, CLIC6 has recently been shown to interact with dopamine D2-like receptors (Griffon N. et al. (2003) Brain Res. Mol. Brian Res. 117(1):47-57). The cell growth regulator CGR11 is novel EF-hand domain proteins which is induced by p53 and has been shown to inhibit the growth of several cell lines. The function of the arginine methyltransferase PRMT2 is not known. Post-translational modification of proteins by arginine methylation has recently been implicated in a variety of cellular processes including nuclear receptor transcriptional regulation. The function of the hypothetical 38.5 kDA protein is not known. Is situ hybridization with a probe specific for this transcript confirmed highly specific expression of this gene that is confined to catecholaminergic neurons (FIG. 16).

The Shared Signature of Midbrain Dopaminergic Neurons

Gene filtering and multiclass significance analysis (Tusher, V. G., et al. (2001) Proc. Natl. Acad. Sci. U.S.A. 98:5116-5121) revealed a set of genes that differ in expression between at least two groups of catecholaminergic neurons (FIG. 14). The genes and experimental samples were grouped based on similarities of gene expression by supervised two-dimensional hierarchical clustering. This analysis again demonstrated the high degree of similarity between SN and VTA dopaminergic neurons, and the distant relationship of the SN/VTA with both A13 dopaminergic and LC neurons.

The SNNTA cluster consisted of genes with enriched expression in both SN and VTA neurons. Aldehyde dehydrogenase 1 (ALDH1A1), which is known to be highly and specifically expressed in these neurons, served as a validating marker for this gene cluster (Galter et al. (2003) Neurobiol Dis. 14(3):637-47). The cluster contained a large number of transcriptional regulators, including the zinc finger-homeodomain proteins ZFH-4 and ATBF1 (Ishii et al. (2003) J. Comp. Neurol. 465(1):57-71; Kostrich et al. (1995) Dev. Dyn. 202(2):145-52), the homeobox factor PBX1, the forkhead-domain family member FOXP2, the interferon-inducible protein IFI 16 and the matrix attachment region binding protein SATB1. Mutations in FOXP2 are linked to severe speech disorders involving the basal ganglia (Liegeois et al. (2003) Nat. Neurosci. 6(11):1230-7). IFI 16 functions as a transcriptional repressor while SATB1 is a modulator of chromatin (Cai et al. (2003) Nat. Genet. 34(1):42-51). A group of regulators of synaptic signaling and/or plasticity included Synaptotagmin I, the calcineurin inhibitor ZAKI-4, the kinesin related protein Hash, the calcium- activated protein for secretion (CAPS), which controls Ca2+-regulated vesicular exocytosis and the glutamate receptor-interacting protein 2 (Grip2) which is involved in the synaptic targeting of AMPA receptors.

A subset of genes that are specifically enriched in SN and VTA (FIG. 14 provides additional examples of genes and gene clusters) are provided in Table 2.

TABLE 2
Examples of targets specifically enriched in SN and VTA neurons

| | GB | Human | | | Ratio | Ratio | Ratio |
|---|---|---|---|---|---|---|---|
| Gene name | accession | Orthologs | Unigene | RatioSN | VTA | A13 | LC |
| Caspase 7 | Y13088 | NM_001227 | Mm.35687 | 11.5 | 10.3 | 0.5 | 0.7 |
| p53 apoptosis associated target (Perp) | AF249870 | NM_022121 | Mm.28209 | 13.1 | 9.4 | 0.7 | 4.5 |
| G-protein coupled thrombin receptor | M81642 | NM_001992 | Rn.2609 | 8.4 | 15.7 | 1.5 | 1.7 |
| Tumor associated Ca²⁺ signal | AJ001044 | NM_002354 | Rn.24930 | 9.2 | 5.1 | 0.7 | 0.3 |
| transducer 1 | | | | | | | |
| Ca2+-dependent activator protein | BC023929 | NM_017954 | Mm.259632 | 10.5 | 9.7 | 0.8 | 0.2 |
| for secretion 2 | | | | | | | |

Three apoptosis related transcripts, Caspase 7, Perp, and SM-20 were highly enriched in SN and VTA neurons. Perp is a positive effector of p53-induced neuronal apoptosis. Moderate levels of Perp were also observed in the LC, while expression was low in hypothalamic dopaminergic neurons which do not degenerate in PD. SM-20 is a mitochondrial protein that promotes caspase-dependent cell death in neurons.

The protease-activated receptor-1 (PAR-1) is a G-protein coupled receptor that is activated by thrombin. Thrombin has been shown to changes the morphology of neurons and astrocytes, and can have cytoprotective or cytotoxic effects on neural cells. The tumor associated Ca²⁺ signal transducer 1 is a cell surface glycoprotein with unknown function that is highly expressed on most human gastrointestinal carcinoma and at a lower level on most normal epithelia but has not been described in brain tissues. Ca2+-dependent activator protein for secretion 2 is a homolog of the CAPS1 protein which is an essential component of the protein machinery involved in large dense-core vesicle exocytosis and in the secretion of a subset of neurotransmitters.

Transcripts Defining LC Noradrenergic Neurons

The LC cluster contained the largest collection of cell group specific transcripts (FIG. 14). Marker genes for this cluster included DBH, monoamine oxidase A and Cytochrome b561, a major transmembrane protein of catecholamine secretory vesicles that provides reducing equivalents for the DBH reaction. AP-2β, a member of the AP-2 family of retinoic acid-induced transcription factors was highly enriched in LC neurons. The closely related AP-2α, which recognizes the same target sequence and shares a highly conserved DNA-binding and dimerization domain, has been shown to activate the expression of TH and DBH (Kim et al. (2001) J. Neurochem. 76(1):280-94) and to be essential for the development of noradrenergic

A subset of targets with high specific expression in LC noradrenergic neurons (see FIG. 14 for additional examples) are provided in Table 3.

TABLE 3
Example of targets with specific expression in LC noragrenergic neurons

| | GB | Human | | | Ratio | Ratio | Ratio |
|---|---|---|---|---|---|---|---|
| Gene name | accession | Orthologs | Unigene | RatioSN | VTA | A13 | LC |
| copper transporter 1 | AF268030 | NM_001859 | Rn.2789 | 2.0 | 2.3 | 1.7 | 39.1 |
| gamma-glutamyltranspeptidase- | U76252 | NM_004121 | Rn.44367 | 0.4 | 0.8 | 1.2 | 10.8 |
| related enzyme | | | | | | | |
| prostaglandin E synthase | AB041998 | NM_004878 | Rn.7730 | 0.5 | 1.0 | 1.2 | 4.0 |
| pigment epithelium-derived factor | AF036164 | NM_002615 | Mm.2044 | 0.1 | 0.2 | 0.2 | 12.7 |

High specific expressions of the copper transporter 1 and the glutathione metabolizing gamma-glutamyltranspeptidase-related enzyme were observed. Copper is an essential cofactor for various enzymes including Cu, Zn superoxide dismutase, cytochrome oxidase and DBH. However, excess of copper combined with glutathione metabolites leads to free radical damage and possible neuronal dysfunction (Enoiu et al. (2000) Free Radic Biol. Med. 29(9):825-33). The expression of the copper transporter 1 in locus coeruleus neurons further supports the view that metal ion transporters play an important role in determining the vulnerability of neuronal populations to neurotoxic stress. Another potential vulnerability factor in LC neurons was prostaglandin E synthase. LC neurons also expressed high levels of pigment epithelium-derived factor (PEDF), a member of the serine protease inhibitor (serpin) family, which is a survival factor for various types of neurons.

Other transcripts that are specifically expressed in LC neurons included the oxygen-binding hemoprotein neuroglobin, the inhibitor of apoptosis proteins (IAPs) and the Tumor Necrosis Factor (TNF) receptor associated factor (TRAF) that mediate the anti-apoptotic signals from TNF. A moderate expression of tryptophan hydroxylase in the noradrenergic neurons of the LC, which has been reported previously (Iijima et al. (1993) Histol Histopathol. 8(3):1387401) was also observed.

Transcripts Defining Hypothalamic A13 Dopaminergic Neurons

The A13 dopaminergic neurons were characterized by high and specific expression of multiple transcriptional regulators (FIG. 14). These included the onecut transcription factor Hnf-6, the LIM-only protein Lmo2, the zinc finger Bteb2 and the homeodomain proteins Isl-1, Nkx2.1, D1x, Six3, Lim1, Prox1 and Arx. Six3 has been shown to alter the regional responses to Fgf8 and Shh, which is required for development of the hypothalamus (Kimura et al. (1996) Genes Dev. 10(1):60-9). The Arx, D1x, Isl-1, Lim1 and Nkx2.1 are important regulators of proliferation, migration and differentiation of neurons in the embryonic forebrain (Kitamura et al. (2002) Nat. Genet. 32(3):359-69). In D1x1/2 mutants for example, the A13 dopaminergic neurons do not form (Andrews et al. (2003)). The functions of Hnf-6, Lmo2, Bteb2, and Prox1 in the A13 dopaminergic neurons are currently not known. The fact that expression of multiple developmental regulators is sustained in adult rat brains, suggests additional yet-to-be identified functions.

Like the SN/VTA neurons, the A13 dopaminergic neurons and the noradrenergic neurons in the LC each also expressed their own characteristic member of the aldehyde dehydrogenase family. The ALDH1A3 family member expressed in the hypothalamus and ALDH1A1 in the SN/VTA can both convert retinaldehyde to retinoic acid. Signaling of retinoic acid was shown to be involved in many developmental processes including the specification of motorneurons sub-classes (Sockanathan and Jessell (1998) Cell 94(4):503-14) and might also be important in the catecholaminergic system. ALDH3A1 expressed in LC neurons is not capable of synthesizing retinoic acid but could be involved in detoxification and the metabolism of neurotransmitters.

Differential Gene Expression in SN and VTA Neurons

Transcripts which are differentially expressed between SN and VTA neurons were identified by two-class significance analysis (FIG. 15). Among these were transcripts from various functional categories including transcriptional regulators (Sox-6, Zfp 288, HTF, NGFI-A), molecules involved in vesicle trafficking (DOC2B, rab3B, MARCKS), axon guidance (neuropilin-1, Slit-2 and Ephrin B3), transporters (VGLUT2, CNT2) and ion channels (CLIC5).

The most prominent gene classes identified encoded factors involved in cell survival and protection which were all expressed at a higher level in the VTA neurons. A subset of such transcripts are provided in Table 4 (see also FIG. 15).

TABLE 4
Examples of targets with differential expression in SN and VTA neurons

| | GB | Human | | | Ratio | Ratio | Ratio |
|---|---|---|---|---|---|---|---|
| Gene name | accession | Orthologs | Unigene | RatioSN | VTA | A13 | LC |
| pituitary adenylate cyclase- | M63006 | NM_001117 | Rn.37400 | 0.2 | 4.0 | 0.3 | 1.4 |
| activating polypeptide | | | | | | | |
| atrial natriuretic peptide | K02062 | NM_006172 | Rn.2004 | 0.5 | 1.8 | 14.1 | 2.9 |
| bone morphogenic protein 2 | L20678 | NM_001200 | Rn.12687 | 1.0 | 2.6 | 0.3 | 1.4 |
| castration induced prostatic | AJ010750 | NM_015393 | Rn.21667 | 1.0 | 5.0 | 7.4 | 1.2 |
| apoptosis-related protein 1 | | | | | | | |
| Extracellular superoxide | Z24721 | NM_003102 | Rn.10358 | 0.5 | 1.2 | 0.4 | 0.4 |
| dismutase | | | | | | | |
| Lipoprotein lipase | L03294 | NM_000237 | Rn.3834 | 0.1 | 2.0 | 0.1 | 0.0 |
| UDP-glucuronyltransferase-S. | AB010441 | NM_080742 | Rn.42869 | 0.6 | 2.6 | 2.5 | 1.2 |
| GPRC5A | NM_181444 | NM_003979 | Mm.23575 | 7.8 | 2.0 | 2.0 | 1.9 |
| Zn²⁺ transporter ZIP-4 | BQ196656 | NM_017767 | Rn.7960 | 103.6 | 265.5 | 1.6 | 1.4 |
| gamma-synuclein | X86789 | NM_003087 | Rn.10421 | 28.1 | 4.3 | 1.8 | 29.5 |
| protein kinase C delta | M18330 | NM_006254 | Rn.98279 | 0.9 | 0.2 | 0.1 | 0.1 |

PACAP and BMP-2 are known survival factors for ventral mesencephalic dopaminergic neurons that can protect from 6-hydroxydopamine and MPTP (Espejo et al., (1999) Neurosci Lett. 275(1):13-6; Reiriz et al. (1999) J. Neurobiol. 38(2):161-70; Takei et al. (1998) J. Neurosci Res. 54(5):698-706). ANP can counteract oxidative stress and excess NO (Vaudry et al. (2002) Eur. J. Neurosci. 15(9):1451-60; Fiscus (2003) Neurosignals 11 (4):175-90). PARM-1 is implicated in suppression of apoptosis (Bruyninx et al. (1999) Endocrinology 140(10):4789-99). The expression BMP-2 was paralleled by the BMP-inducible antagonists follistatin and chordin, which is indicative of active BMP-signaling in adult VTA neurons controlled by autoregulatory feedback loops.

Enriched expression in VTA over SN neurons was also observed for enzymes with detoxifying properties. See Table 4 and FIG. 15. Extracellular superoxide dismutase is an antioxidant enzyme that attenuates brain and lung injury from oxidative stress (Sheng et al. (2000) Exp. Neurol. 163(2):392-8). Lipoprotein lipase is a key enzyme involved in the metabolism of lipoproteins, which protects from cell death induced by oxidized lipoproteins (Paradis et al. (2003) J. Biol. Chem. 278(11):9698-705). UDP-glucuronosyltransferase detoxifies compounds by conjugation to glucuronic acid. On the other hand, expression of PKC-delta, a potent promoter of neurodegeneration, was significantly lower in VTA neurons compared to the SN. Proteolytic activation of PKC delta has been shown to mediate dopaminergic neuronal cell apoptosis induced by MPTP or pesticides (Kaur et al. (2003) Neuron 37:899-909; Kitazawa et al. (2003) Neuroscience 119(4):954-64). High expression of gamma-synuclein in neurons of the SN compared to the VTA was also observed. gamma-synuclein transcripts are highly enriched in both, SN and LC (28 and 29-fold respectively), which are vulnerable to PD and is dramatically lower in the less vulnerable VTA (4 fold) and A13 (2 fold) neurons and it may contribute to the SN and LC specific toxic effects of the widely expressed a-synuclein protein. Retinoic acid induced 3 (RAI3 or GPRCSA) is an orphan G protein-coupled receptor with unknown function that is induced by retinoic acid. This gene is a member of the type 3 G protein-coupling receptor family, characterized by the signature 7-transmembrane domain motif and may be involved in interaction between retinoid acid and G protein signalling pathways.

The Zn²⁺ transporter ZIP-4 was also dramatically enriched in the SN (>100 fold) and the VTA (>250 fold) but not in A13 or LC. The specific expression of ZIP-4 was confirmed by in situ hybridization (FIG. 16). Zn²⁺ ions could play a role in the pathophysiology of Parkinson's disease. Metal ions increase oxidative damage following energy failure in the cells. Parkinson research has emphasized Fe²⁺ because of the high concentration of this metal ion in the substantial nigra. Chelators of Fe²⁺ prevent the toxic effects of MPTP on dopaminergic neurons (Kauer I., (2003) Neuron 37(4):549-50). Zn²⁺ has been the focus of attention in neurodegeneration in the hippocampus following ischemic stroke. The granule cells contain high levels of Zn²⁺ that is released synaptically and able to damage postsynaptic neurons at high concentrations (Sloviter, (1985) Brain Res. 330(1): 150-3). Subsequent studies confirmed the toxicity of Zn²⁺ in cell culture systems., Zn²⁺ can also act as an inhibitor of cell death if present at low concentrations. Based on the findings presented here the Zn²⁺ transporter ZIP-4 could have an important role in dopaminergic neurotoxicity and could be useful as a drug target.

Other transcripts that were highly enriched in SN and VTA neurons include factors with a reported or anticipated function in synaptic plasticity, including the synaptic adhesion molecules synCAM and syndecan-2 (Yamagata (2003) Curr. Opin. Cell Biol. 15(5):621-32) and the actin-associated synaptopodin-2 which belongs to a class of factors required for the formation of the spine apparatus in dendritic spines, an important site of neuronal plasticity (Deller et al. (2003) Proc. Natl. Acad. Sci. USA 100(18):10494-9). The myristoylated alanine-rich C kinase substrate (MARCKS) and G-substrate are substrates of protein kinase C and cGMP-dependent protein kinase respectively and have been implicated in learning and long-term potentiation (LTP). Phospholipase-Cγ (PLCγ) is suspected to be involved in the maintenance of LTP (Ernfors and Bramham (2003) Trends Neurosci. 26(4):171-3) while NGFI-A or Zif268 is an immediate early gene associated with learning and plasticity. The serine proteases, RNK-Met 1 and DISP as well as the serine protease inhibitor Hai2 which might contribute to synaptic plasticity by modulation of the extracellular environment were also identified.

This study analyzed the molecular signatures that define the major subpopulations of CA neurons. It was shown that individual neurons can be identified by a rapid immunostaining protocol and isolated from brain tissue with an intact complement of RNA that is suited for amplification and microarray analysis. Phylogenetic analysis revealed a very close relationship between midbrain DA neurons in the SN and the VTA. Despite considerable heterogeneity in the mesotelencephalic DA system with respect to cell morphology, target innervation, electrophysiological properties, and disease susceptibility, this study determined differential expression of less than 1% of their genes. In contrast, 5% of the transcripts in the hypothalamic DA neurons differed from these of the SN or VTA neurons. DA neurons in the midbrain and hypothalamus each expressed their own specific sets of transcriptional regulators. This suggests that the DA phenotype in these groups of neurons could be maintained, at least in part, by independent regulatory cascades. In fact, while midbrain and forebrain DA neurons depend on the same signaling molecules (FGF 8 and Shh) during early development, several factors have been identified that selectively control DA fate in the midbrain (Nurr1, Lmx1b, Pitx3).

The fourth cell group analyzed, the NA neurons in the LC, displayed differences in transcripts of about 7% when compared to the DA SN or VTA and of more than 10% compared to the hypothalamic A13 cell group. In LC NA neurons, the expression of dopamine synthesizing enzymes seems to be controlled by a different transcription factors than in the DA cell groups (Goridis and Rohrer (2002) Nat. Rev. Neurosci. 3(7):531-41). In contrast to DA neurons in the midbrain and hypothalamus, only a single transcription factor, AP-2β, with LC specific expression was identified. The closely related family member AP-2α, which was not present on the array, has recently been shown to activate the TH and DBH promoters (Kim et al. (2001) J. Neurochem. 76(1):280-94) and to be required for the development of LC neurons in zebrafish embryos (Holzschuh at al. (2003) Development 130(23):5741-54). The precise role of AP-2β in NA neurons which recognizes the same target sequence and can heterodimerize with AP-2 α is not known.

The complexity of cell group specific gene expression seems to be correlated with the diversity of projections and the complexity of biological functions of the individual CA subclasses. The LC NA system, which provides a highly divergent innervation to virtually the entire CNS, allowing it to regulate emotional, cognitive and sleep-wake functions, expressed the highest number of specific genes. In contrast, hypothalamic A13 neurons which have a less extensive network of projections and control less diverse brain functions, expressed less than half that number of specific genes, while SN and VTA had intermediate numbers of enriched transcripts.

Despite the high similarity of the transcriptomes in SN and VTA neurons, a number of subpopulation-specific genes were identified. Among the gene transcripts enriched in the VTA were several encoding synaptic plasticity proteins such as PLCγ, synCAM, syndecan-2, synaptopodin-2, MARCKS, G-substrate, and Zif268. These could may contribute to the long-term synaptic plasticity elicited by psychostimulants leading to drug addiction (Gerdemann et al. (2003) Trends Neurosci. 26(4):184-92). A critical role of PLCγ in the regulation of long-term adaptations to drugs has recently been demonstrated by overexpression experiments in the VTA (Bolanos et al. (2003) J. Neurosci. 23(20):7569-76). Likewise, the expression of the learning and plasticity-associated immediate early gene Zif268 is induced in VTA neurons upon drug-conditioned stimulation and decreases during prolonged withdrawal (Thomas et al. (2003) Eur. J. Neurosci 17(9):1964-72; Mutschler et al (2000) Neuroscience 100(3):531-8).

VTA neurons were also enriched in several factors involved in axonal pathfinding and neuronal migration (neuropilin-1, slit-2 and ephrin B3). During development, SN neurons target mainly the dorso-lateral striatum while VTA neurons mainly innervate the ventromedial striatum, constituting mesostriatal and mesolimbic pathways respectively. The molecular signals that regulate the development of these pathways have only been partially characterized (Yue et al. (1999) J. Neurosci 19(6):2090-101) and differential expression of multiple members of the ephrin/Eph and slit/robo family identified here could have important functions in path finding and adult plasticity. These findings are particularly interesting from a point of view of schizophrenia, a disease most likely linked to abnormal development of cortical areas innervated by the VTA neurons (Lewis and Levitt (2002) Neurosci. 25:409-432). DISC1, the first discovered schizophrenia gene, is expressed at highest levels in the cortex during development. It interacts with NudE-like (NUDEL) earlier linked to cortical development (Ozeki et al. (2003) Proc. Natl. Acad. Sci. USA 100:289-294). Linkage studies have identified neuregulin 1 as a susceptibility gene in Islandic and Scottish populations (Stefansson et al. (2003) Am. J. Hum. Genet. 72:83-87). Neuregulin is a member of a multigene family of transmembrane proteins that contain an extracellular EGF-like domain necessary for function and which play an important role in the developmental of neurons and glial cells. A further schizophrenia susceptibility gene identified by linkage studies is dystrobrevin-binding protein 1, a protein is contained in postsynaptic densities and functionally linked to synaptic plasticity (Straub et al. (2002) Am. J. Hum. Genet. 71:337-348). These findings are compatible with the view that schizophrenia is, at least in part, a developmental disorder of the development of the cortex. The genes identified in this study as selectively expressed by VTA DA neurons could participate in the disease-related pathways of schizophrenia.

A goal of this analysis was to identify genes that may influence the selective vulnerability catecholiminergic (CA) neurons in Parkinson's Disease (PD). The subpopulation of dopamine neurons confined to the zona compacta of the substantia nigra are most susceptible to Parkinson's disease pathology. Their degeneration causes the vast majority of behavioral symptoms of the disease. The adjacent VTA dopamine neurons are less vulnerable, and hypothalamic DA neurons are spared (Farneley and Lees (1991) Brain 114 (Pt 5): 2283-2301; Hirsch et al., (1988) Nature 334:345-348; Uhl et al. (1985) Neurology 35(8):1215-8; Purba et al. (1994) Neurology 44(1):84-9; Matzuk et al., (1985) Ann Neurol 5:552-5). The same selective vulnerability of DA neuron subpopulations has been observed in rodent and primate models of PD (Melamed et al. (1985) Eur. J. Pharmacol. 114(1):97-100; Mogi et al. (1988) J. Neurochem. 50(4):1053-6; Zuddas et al. (1989); Varastet et al. (1993) Neuroscience 63(1):47-56). Based on the expression of genes known to counteract stress-induced cell damage (glutathione peroxidase, 8-oxo-dGTPase, ARC, ORP150), it appears that all CA cell groups are under oxidative stress possibly resulting from DA metabolism. The selective vulnerability could reside in the multiple cell group specific transcripts for regulators of oxidative stress, excitotoxicity, apoptosis, mitochondrial dysfunction and neuroinflammation that we have identified. For instance, significance analysis identified a group of VTA-enriched neuroprotective factors including neurotrophic factors (BMP-2, PACAP, ANP), detoxifying enzymes (EC-SOD, lipoprotein lipase, UDP-glucuronosyltransferase), the anti-apoptotic factor PARM-1 and decreased levels of the pro-apoptotic PKC delta that may account for the sparing of VTA neurons in PD. High expression of gamma-synuclein in neurons of the SN and in LC noradrenergic neurons that degenerate in PD compared to the VTA and A13 which could contribute to the SN and LC specific toxic effects of the widely expressed gamma-synuclein protein was also observed.

The selective expression of the Zn²⁺ transporter by the SN and VTA suggests that the possibility that this ion plays a role in the pathophysiology of Parkinson's disease. Metal ions increase oxidative damage following energy failure in the cells. Parkinson research has emphasized Fe²⁺ because of the high concentration of this metal ion in the substantial nigra. Chelators of Fe²⁺ prevent the toxic effects of MPTP on DA neurons (Kaur et al. (2003) Neuron 37:899-909). Zn²⁺ has been the focus of attention in neurodegeneration in the hippocampus following ischemic stroke. The granule cells contain high levels of Zn²⁺ that is released synaptically and able to damage postsynaptic neurons at high concentrations (Sloviter (1985) Brain Res. 330:150-153). Subsequent studies confirmed the toxicity of Zn²⁺ in cell culture systems. The findings suggest that Zn²⁺ could be equally important for DA neurotoxicity. The expression of the copper transporter 1 in locus coeruleus neurons further supports the view that metal ion transporters play an important but complex role in determining the vulnerability of neuronal populations to neurotoxic stress.

The findings herein, provide the first genomic analysis of clinically relevant classes of CA neurons revealing previously unrecognized patterns of gene expression that are shared or confined to specific populations of CA neurons. The data leads to better understanding of the distinct features and functions of these groups of neurons and provides drug targets that could be useful for drug development. For example, the drug targets presented in Tables 1-4, could be useful for Parkinson's disease.

Although the foregoing invention has been described in some detail by way of illustration and example for purposes of clarity of understanding, it will be apparent to those skilled in the art that certain changes and modifications may be practiced. Therefore, the descriptions and examples should not be construed as limiting the scope of the invention.

1. A method of identifying a candidate drug target in a population of dopaminergic or noradrenergic neurons comprising evaluating the expression of one or more polynucleotides in a dopaminergic or noradrenergic neuron population, wherein the one or more polynucleotides are candidate drug targets.

2. A method of identifying candidate drug targets in a population of dopaminergic or noradrenergic neurons comprising: (a) identifying the population of neurons; (b) isolating the populations of neurons; (c) evaluating the expression of one or more polynucleotides in the population of neurons, wherein the one or more polynucleotides and/or the one or more encoded polypeptides are candidate drug targets.

3. The method of claim 1 or claim 2, further comprising the step of evaluating the expression of one or more polynucleotides in step (b) relative to a control population of neurons.

4. The method of claim 3, wherein the control population of neurons is from whole brain.

5. The method of claim 1 or claim 2, wherein the population of neurons are dopaminergic neurons.

6. The method of claim 1 or claim 2, wherein the population of neurons are noradrenergic neurons.

7. The method of claim 5, wherein the population of dopaminergic neurons are substantia nigra neurons.

8. The method of claim 5, wherein the dopaminergic neurons are ventral tegmental area neurons.

9. The method of claim 5, wherein the dopaminergic neurons are zona encarta (A13 group) neurons.

10. The method of claim 6, wherein the noradrenergic neurons are locus coeruleus neurons.

11. The method of claim 1 or claim 2, wherein the population of neurons are obtained from a pathology sample, an autopsy sample, a biopsy sample, a brain tissue bank or in vitro cultures of dopaminergic or noradrenergic neurons.

12. The method of claim 1 or claim 2, wherein the population of neurons are human neurons or rodent neurons.

13. The method of claim 1 or claim 2, wherein the level of expression of the gene transcript corresponding to the drug target is evaluated.

14. The method of claim 1 or claim 2, wherein the level of expression of the polypeptide corresponding to the drug target is evaluated.

15. A method of assessing the ability of a candidate agent to modulate dopaminergic or noradrenergic neuron activity and/or function comprising measuring the level of expression of the one or more drug targets selected by the method of claim 1 or claim 2, wherein an alteration of the level of expression of the one or more drug targets indicates the ability of the candidate agent to modulate dopaminergic and/or noradrenergic neuron activity and/or function.

16. A method of assessing the ability of a candidate agent to modulate dopaminergic or noradrenergic neuron activity or function comprising: (a) contacting a population of dopaminergic and/or noradrenergic neurons expressing one or more drug targets with a candidate agent, wherein the one or more drug targets are selected from the drug targets referenced in FIG. 5, FIG. 6, FIG. 7, FIG. 8, FIG. 9, FIG. 10, FIG. 11, FIG. 12, FIG. 13, FIG. 14, FIG. 15, Table 1, Table 2, Table 3 or Table 4 or combinations thereof and (b) measuring the level of expression of the one or more drug targets in the population of dopaminergic or noradrenergic neurons, wherein an alteration of the level of expression of the one or more drug targets indicates the ability of the candidate agent to modulate dopaminergic or noradrenergic neuron activity or function.

17. The method of claim 16, wherein the population of neurons are substania nigra dopaminergic neurons and the one or more drug targets are selected from FIG. 8, FIG. 9, FIG. 10, FIG. 11, FIG. 13, FIG. 14 FIG. 15, Table 1, Table 2 or Table 4 or combinations thereof.

18. The method of claim 16, wherein the population of neurons are zona incerta A13 neurons and the one or more drug targets are selected from FIG. 5, FIG. 13, FIG. 14 or Table 1 or combinations thereof.

19. The method of claim 16, wherein the population of neurons are ventral tegmental area neurons and the one or more drug targets are selected from FIG. 7, FIG. 9, FIG. 10, FIG. 13, FIG. 14, FIG. 15, Table 1, Table 2 or Table 4 or combinations thereof.

20. The method of claim 16, wherein the population of neurons are locus coeruleus neurons and the one or more drug targets are selected from FIG. 6, FIG. 12, FIG. 13, FIG. 14, Table 1 or Table 3 or combinations thereof.

21. The method of any one of claims 16-20, wherein the level of expression of the gene transcript corresponding to the drug target is measured.

22. The method of any one of claims 16-20, wherein the level of expression of the polypeptide corresponding to the drug target is measured.

23. A method of assessing the ability of a candidate agent to modulate dopaminergic or noradrenergic neuron activity and/or function comprising: (a) contacting a population of dopaminergic and/or noradrenergic neurons expressing one or more drug targets with a candidate agent, wherein the one or more drug targets are selected from the drug targets referenced in Table 1, Table 2, Table 3 or Table 4 and (b) measuring the level of expression of the one or more drug targets in the population of dopaminergic and/or noradrenergic neurons, wherein an alteration of the level of expression of the one or more drug targets indicates the ability of the candidate agent to modulate dopaminergic or noradrenergic neuron activity and/or function.

24. The method of claim 23, wherein the one or more drug targets are selected from Table 1.

25. The method of claim 23, wherein the population of neurons are substania nigra dopaminergic neurons and the one or more drug targets are selected from Table 2 or Table 4 or combinations thereof.

26. The method of claim 23, wherein the population of neurons are ventral tegmental area neurons and the one or more drug targets are selected from Table 2 or Table 4 or combinations thereof.

27. The method of claim 23, wherein the population of neurons are locus coeruleus neurons and the one or more drug targets are selected from Table 3.

28. The method of claim 23, wherein the population of neurons are locus coeruleus neurons and the one or more drug targets are selected from Table 1 and 3.

29. The method of claim 23, wherein the population of neurons are zona incerta A13 neuron neurons and the one or more drug targets are selected from Table 1.

30. The method of any one of claims 23-29, wherein the level of expression of the gene transcript corresponding to the drug target is measured.

31. The method of any one of claims 23-29, wherein the level of expression of the polypeptide corresponding to the drug target is measured.

32. A method of assessing the ability of a candidate agent to bind to one or more drug targets for dopaminergic or noradrenergic neurons, said method comprising: (a) contacting the one or more drug targets selected from FIG. 5, FIG. 6, FIG. 7, FIG. 8, FIG. 9, FIG. 10, FIG. 11, FIG. 12, FIG. 13, FIG. 14, FIG. 15, Table 1, Table 2, Table 3 or Table 4 or combinations thereof with a candidate agent and (b) evaluating the binding of the candidate agent to the drug target, wherein the ability of the candidate agent to bind to the drug target is indicative of the possible therapeutic potential of the candidate agent.

33. The method of claim 32, wherein the neurons are substania nigra dopaminergic neurons and the one or more drug targets are selected from FIG. 8, FIG. 9, FIG. 10, FIG. 11, FIG. 13, FIG. 14 FIG. 15, Table 1, Table 2 or Table 4 or combinations thereof.

34. The method of claim 32, wherein the neurons are zona incerta A13 neurons and the one or more drug targets are selected from FIG. 5, FIG. 13, FIG. 14 or Table 1 or combinations thereof.

35. The method of claim 32, wherein the neurons are ventral tegmental area neurons and the one or more drug targets are selected from FIG. 7, FIG. 9, FIG. 10, FIG. 13, FIG. 14, FIG. 15, Table 1, Table 2 or Table 4 or combinations thereof.

36. The method of claim 32, wherein the neurons are locus coeruleus neurons and the one or more drug targets are selected from FIG. 6, FIG. 12, FIG. 13, FIG. 14, Table 1 or Table 3 or combinations thereof.

37. The method of claim 32, wherein the drug target is a polynucleotide drug target.

38. The method of claim 32, wherein the drug target is a polypeptide.

39. The method of claim 32, wherein the level of expression of the gene transcript corresponding to the drug target is measured.

40. The method of claim 32, wherein the level of expression of the polypeptide corresponding to the drug target is measured.

41. The method of any one of claims 32-40, wherein the candidate agent is an antibody.

42. A microarray comprising polynucleotide drug targets for substantia nigra neurons, the microarray comprising one or more of the polynucleotide drug targets or fragments thereof referenced in FIG. 8, FIG. 9, FIG. 10, FIG. 11, FIG. 13, FIG. 14, FIG. 15, Table 1, Table 2 or Table 4.

43. A microarray comprising polynucleotide drug targets for zona incerta A13 neurons, the microarray comprising one or more drug targets are selected from FIG. 5, FIG. 13, FIG. 14 or Table 1.

44. A microarray comprising polynucleotide drug targets for ventral tegmental area neurons, the microarray comprising one or more of the polynucleotide drug targets or fragments thereof referenced in FIG. 7, FIG. 9, FIG. 10, FIG. 13, FIG. 14, FIG. 15, Table 1, Table 2 or Table or 4.

45. A microarray comprising polynucleotide drug targets for locus coeruleus neurons, the microarray comprising one or more of the polynucleotide drug targets or fragments thereof referenced in FIG. 6, FIG. 12, FIG. 13, FIG. 14, Table 1 or Table 3.

46. A microarray comprising at least 2, 3, 5, 10, 20, 40, 50, 60, 70, 80, 90, 100, 200, 300 or 400 of the polynucleotide drug targets in claims 42-45or combinations thereof.

47. A microarray comprising at least 2, 3, 5, 10, 20, 40, 50, 60, 70, 80, 90, 100, 200, 300 or 400 of the polynucleotide drug targets referenced in FIGS. 5-15 and Tables 1-4.

48. The microarray of claim 47 comprising at least 20, 30, 40 or 50 of the polynucleotide drug targets referenced in FIGS. 5-15 and Tables 1-4.

49. The microarray of claim 47 comprising at least 60, 70, 80, 90, 100, 200, 300 or 400 of the polynucleotide drug targets referenced in FIGS. 5-15 and Tables 1-4.

50. A microarray comprising polypeptide drug targets for substantia nigra neurons, the microarray comprising one or more of the polypeptide drug targets or fragments thereof encoded by a polynucleotide referenced in FIG. 8, FIG. 9, FIG. 10, FIG. 11, FIG. 13, FIG. 14, FIG. 15, Table 1, Table 2 or Table 4.

51. A microarray comprising polypeptide drug targets for zona incerta A13 neurons, the microarray comprising one or more of the one or more of the polypeptide drug targets or fragments thereof encoded by a polynucleotide referenced in FIG. 5, FIG. 13, FIG. 14 or Table 1.

52. A microarray comprising polypeptide drug targets for ventral tegmental area neurons, the microarray comprising one or more of the polypeptide drug targets or fragments thereof encoded by a polynucleotide referenced in FIG. 7, FIG. 9, FIG. 10, FIG. 13, FIG. 14, FIG. 15, Table 1, Table 2 or Table or 4.

53. A microarray comprising polypeptide drug targets for locus coeruleus neurons, the microarray comprising one or more of the polypeptide drug targets or fragments thereof encoded by a polynucleotide referenced in FIG. 6, FIG. 12, FIG. 13, FIG. 14, Table 1 or Table 3.

54. A microarray comprising at least 2, 3, 5, 10, 20, 40, 50, 60, 70, 80, 90, 100, 200, 300 or 400 of the polypeptide drug targets in claims 50-53 or combinations thereof.

55. A microarray comprising at least 2, 3, 5, 10, 20, 40, 50, 60, 70, 80, 90, 100, 200, 300 or 400 of the polypeptide drug targets encoded by the polynucleotide drug targets referenced in FIGS. 5-15 and Tables 1-4.

56. A kit comprising one or more microarrays of any of claims 42-55.
